(19) United States
(12) Patent Application Publication
(10) Pub. No.: US 2022/0287967 A1
(43) Pub. Date: Sep. 15, 2022

(54) EXOSOME MIMICKING NANOVESICLES MAKING AND BIOLOGICAL USE

(71) Applicants: Harsha JYOTHI, Davis, CA (US); Lalithasri RAMASUBRAMANIAN, Davis, CA (US); The Regents of the University of California, Oakland, CA (US)

(72) Inventors: Harsha Jyothi, Davis, CA (US); Lalithasri Ramasubramanian, Davis, CA (US); Aijun Wang, Davis, CA (US)

(73) Assignee: The Regents of the University of California, Oakland, CA (US)

(21) Appl. No.: 17/604,375

(22) PCT Filed: Apr. 17, 2020
(86) PCT No.: PCT/US2020/028867

§ 371 Date: Oct. 15, 2021

Related U.S. Application Data
(60) Provisional application No. 62/836,028, filed on Apr. 18, 2019.

(51) Int. Cl. A61K 9/127; A61K 35/28; A61K 47/69; A61P 25/28; A61P 25/16; A61P 9/10

(52) U.S. Cl. CPC A61K 9/1271; A61K 35/28; A61K 47/6911; A61P 25/28; A61K 9/1278; A61P 25/16; A61P 9/10

(57) ABSTRACT

This disclosure provides an exosome mimicking nanovesicle (EMN) comprising cell-derived plasma membrane or lipid rafts and substantially devoid of native exosomes. The EMNs can be derived from a differentiated cell or a stem cell. They are useful to carry a variety of cargo, e.g., a secretome, or exogenous agent selected from a polynucleotide, a peptide, a protein, an antibody fragment, a chemical, or a therapeutic agent. They are useful for the treatment of a variety of diseases and disorders.

### CROSS-REFERENCE TO RELATED APPLICATION

This application claims priority under 35 U.S.C. § 119(e) to U.S. Provisional Application Ser. No. 62/836,028 filed Apr. 18, 2019, the contents of which are each incorporated by reference into the present disclosure.

### SEQUENCE LISTING

The instant application contains a Sequence Listing which has been submitted electronically in ASCII format and is hereby incorporated by reference in its entirety. Said ASCII copy, created on Apr. 17, 2020, is named 060933-9360_SequenceListing_ST25.txt and is 4096 bytes in size.

### BACKGROUND

Neurological disorders are devastating and affect the daily activities of millions of people globally. A number of neurological diseases lead to neurodegeneration characterized by irreversible damage or loss of neurons located in the central nervous system.

Over 100 million Americans suffer from neurological diseases. The United States spends an estimated $800 billion every year for the care of individuals with neurological diseases. Shaw et al. (2017) published breakdown costs for the treatment of neurological disorders in the USA, such as $227 billion for Alzheimer's Disease and others, $177 billion for chronic low back pain, $110 billion for stroke, $86 billion for traumatic brain injury, $78 billion for migraine headache, $37 billion for epilepsy, and $25 billion for multiple sclerosis.

Current treatment options can only help manage the symptoms due to the complexity of neurological diseases. Thus, a need exists in the art for more effective therapies for neurological and other complex disorders, e.g., vascular disease and autoimmune disorders. This disclosure satisfies this need and provides related advantages as well.

### SUMMARY OF THE DISCLOSURE

Due to the complexity of neurological diseases, vascular diseases, autoimmune disorders, current treatment options for these and other disorders are limited in that they only manage symptoms. Current therapies also fall short of halting or reversing the sequential neuronal damage, thus warranting the need for other effective treatments.

Several clinical studies are focused on mesenchymal stromal cells as potential therapies because of their ability to differentiate to other cell forms (for example osteocytes, endothelial cells, chondrocytes, adipocytes, odontoblasts and neurons) and their ability to renew themselves. In addition, they are not recognized by the immune system as foreign. See, e.g., Oh et al. (2015). In addition, mesenchymal stromal cells (MSCs) also possess immunomodulatory properties and have potential in repairing myelomeningocele (MMC). See, for example, Guo et al. (2017) and Chen et al. (2017).

Several treatments are under investigation in combatting autoimmune diseases and/or hyper-inflammation, such as use of MSCs (see, for example, Klinker et al. (2015)).

Extracellular vesicles (EVs) are small nanovesicles derived from the invagination of the cell plasma membranes (PM) that function as primary messengers of intercellular communication (Théry et al. (2002), Colombo et al. (2014)). EVs can be secreted by all types of cells that have shown great promise as noninvasive nanotherapeutics for regenerative medicine (Théry et al. (2002), Colombo et al. (2014)). One of such cells is MSCs which are extensively studied due to proven differentiation, self-renewal and immunomodulatory, angiogenic and neuroprotective properties. All in all, EVs present as a biological and multifunctional therapeutic and treatment for a variety of diseases and defects.

However, EV application for clinical translation has been greatly limited due to difficulties in EV isolation and purification. Obtaining EVs, especially from cell cultures, is an extremely time-consuming, laborious, and costly process. Preferentially sorting functional subpopulations of therapeutic EVs from other vesicle types is also difficult, and thus the isolated EV fractions may contain unwanted populations of vesicles. Additionally, there is an inherent heterogeneity of functional properties between EVs of similar cell origin due to the sensitivity of EV secretion and properties in response to cellular environment (Théry et al. (2002), Colombo et al. (2014)). With the difficulties in obtaining and standardizing EVs, the therapeutic application of these nanovesicles has become a major challenge.

As nano-sized EVs, exosomes have the potential to be effective therapeutic agents. However, prior art EVs have variable composition and their isolation process is time-consuming and their yields are often low. Therefore, an alternative solution to overcome the aforementioned challenges is necessary.

MSCs have been studied due to proven self-renewal and immunomodulatory, angiogenic and neuroprotective properties. Applicant's research has revealed that analyses of MSC secretion attributed these properties to paracrine secretions such as unique cytokines, growth factors and EVs, including exosomes. For example, human placental-derived MSCs (hPMSCs) secrete significant levels of paracrine factors such as hepatocyte growth factor (HGF), brain-derived neurotropic factor (BDNF) and vascular endothelial growth factors (VEGF). Treatment of surgically created fetal lamb myelomeningocele with PMSCs preserved motor neurons and improved their ambulatory functions through paracrine secretion mechanisms. Studies by the Applicant also showed that exosomes secreted by PMSCs, present in the conditioned medium, are effective in alleviating the severity of neuronal damage. See, for example, Zhang et al. (2019), Kumar et al. (2019), and Clark et al. (2019). Since cells can confer their functions via paracrine secretion which contains exosomes, exosomes are an excellent candidate for cell-free therapy as it is biocompatible and facilitates targeted delivery.

Since the membrane composition is similar to that of the plasma membrane (PM), in one aspect, provided is an exosome mimicking nanovesicle (EMN) comprising a shell encapsulating a cargo. In one embodiment, the shell comprises, or consists essentially of, or yet further consists of a plasma membrane. Additionally or alternatively, the shell and/or the EMN comprises, or consists essentially of, or yet further consists of a lipid raft. In a further embodiment, the EMN is substantially devoid of (or substantially free of) native exosomes. In one embodiment, the shell is derived from or isolated from a cell capable of secreting an exosome. In yet a further embodiment, the EMN comprises, or consists essentially of, or yet further consists of a core encapsulated in the shell with the cargo. Additionally or alternatively, the shell further comprises, or consists essentially of, or yet further consists of a peptide or a protein, which can be referred to herein as a shell peptide or a shell protein. In one embodiment, the EMN further comprises, or consists essentially of, or yet further consists or a scaffold.

In certain embodiments, a cargo of the EMN as disclosed herein comprises, or consists essentially of, or yet further consists of an exogenous agent. In a further embodiment, the exogenous agent is selected from a polynucleotide, a peptide, a protein, an antibody fragment, a small molecule or a therapeutic agent.

Non-limiting examples of polynucleotides include a RNA, a DNA, an inhibitory RNA, an miRNA, an siRNA, a therapeutic gene or a CRISPR system. In a further embodiment, the miRNA is one or more of the following: hsa-miR-138-5p, hsa-miR-22-5p, miR-218-5p, hsa-let-7b-5p, hsa-let-7f-5p, hsa-miR-122-5p, hsa-let-7g-5p, hsa-let-7i-5p, hsa-miR-22-5p, hsa-miR-186-5p, hsa-let-7d-5p, hsa-miR-19a-3p, hsa-mir-98, hsa-let-7c, or hsa-miR-29a-3p. In yet a further embodiment, the cargo comprises an miRNA and a cationic counterion (such as spermidine). In one embodiment, the cargo comprises, or consists essentially of, or yet further consists of a complex comprising an hsa-miR126-3p and a cationic counterion (such as spermidine).

In one embodiment, the cargo comprises a peptide or a protein that is optionally selected from one or more of a growth factor, a chemokine, or a cytokine. In a further embodiment, the growth factor is selected from the group of: a platelet-derived growth factor, a hepatocyte growth factor (HGF), a brain-derived neurotropic factor (BDNF), or a vascular endothelial growth factors (VEGF) or a combination thereof. In yet a further embodiment, the chemokine or cytokine is selected from the group of: a monocyte chemoattractant protein-1 (MCP-1), IL-8, or IL-6 or a combination thereof. Additionally or alternatively, the cargo comprises a peptide or a protein that optionally selected from the group of: HGF, BDNF, VEGF, galectin 1, MCP-1, IL-8, IL-6, a-catenin, b-catenin, platelet-derived growth factor, TGF-β, Wnt5a, tissue factor, integrin a4b1, MMP1, MMP2, MMP14, ADAM9, ADAM10, ADAM17, a disintegrin and metalloprotease (for example, ADAM), matrix metalloproteinase (MMP), or TIMP (optionally a tissue inhibitor of metalloproteinase, for example TIMP 1, TIMP-2, or TIMP-3) BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TNF-α, or a combination thereof.

In certain embodiments, the cargo comprises, or consists essentially of, or yet further consists of a cell derived conditioned medium. In a further embodiment, the conditioned medium comprises, or consists essentially of, or yet further consists of one or more of the following: HGF, BDNF, VEGF, BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TGF-β, or TNF-α.

In certain embodiments, the core comprises, or consists essentially of, or yet further consists of a polymer core, such as for example, one or more of poly(l-lysine) (PLL), polyethylenimine (PEI), polyamidoamines, polyimidazoles, poly(ethylene oxide), polyalkylcyanoacrylates, polylactide, polylactic acid (PLA), poly-ε-caprolactone (PCL), poly (lactic-co-glycolic acid) (PLGA), silica, alginate, cellulose, pullulan, gelatin, or chitosan.

In certain embodiments, the cargo and/or shell comprises a peptide or a protein that is optionally one or more peptide or protein selected from the group of: HGF, BDNF, VEGF, galectin 1, MCP-1, IL-8, IL-6, a-catenin, b-catenin, platelet-derived growth factor, TGF-β, Wnt5a, tissue factor, integrin a4b1, MMP1, MMP2, MMP14, ADAM9, ADAM10, ADAM17, a disintegrin and metalloprotease (for example, ADAM), matrix metalloproteinase (MMP), or TIMP (optionally a tissue inhibitor of metalloproteinase, for example TIMP 1, TIMP-2, or TIMP-3) BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TNF-α, or a combination thereof.

In certain embodiments, the shell peptide or protein facilitates one or more of the following: targeting the EMN to a cell and/or tissue, penetrating a cell, modulating immunoregulatory activity, or protecting a cell. In a further embodiment, the shell peptide or protein is selected from the following: a collagen-binding ligand, a platelet-receptor for collagen, an inhibitor of platelet reactivity, SILY (RRANAALKAGELYKSILYGC, SEQ ID NO: 1), CD39; a cell-penetrating peptide; a cell-targeting peptide; a human leukocyte antigen-G (HLA-G); Galectin1 or a combination thereof. In yet a further embodiment, the peptide or protein is conjugated to the shell covalently or non-covalently, directly or indirectly via a linker.

In one embodiment, provided is an EMN comprising lipid rafts derived from human placenta MSCs (hPMSCs). In one aspect, the shell and hPMSCs-derived conditioned medium are encapsulated as cargos. In another embodiment, provided is an EMN comprising endothelial progenitor cell (EPC) derived plasma membrane in and/or as the shell and miR126 as a cargo, and optionally wherein the cargo is loaded to a PLGA core before encapsulated by the shell.

In one embodiment, the plurality further comprises EMNs comprising serum albumin and/or biotin as the cargo. In another aspect, the shells and/or the cargos are detectably labeled.

In another aspect, provided herein is a composition comprising, or consisting essentially of, or yet further consisting of a carrier and an EMN as disclosed herein. In one aspect the EMNs of the composition and/or a plurality of EMNs are the same or different from each other, and are selected for the specific therapy or diagnostic use. In certain embodiments, the shells or cargos are the same or different from each other. In another embodiment, the shells and cargos are the same or different from each other. In one embodiment, the plurality further comprises EMNs comprising serum albumin and/or biotin as the cargo.

In yet another aspect, provided is a method for rescuing a cell comprising, or consisting essentially of, or yet further consisting of, contacting the cell with or administering an effective amount of an EMN as disclosed herein, and/or a plurality of the EMN as disclosed herein. In one embodiment, the cell is selected from the group of a neuron, an endothelial cell, a cardiomyocyte, a myogenic cell, a smooth muscle cell, or a lung cell. Additionally or alternatively, the administration or contacting is in vitro or in vivo. In another embodiment, the administration is in vivo and the cell is a mammalian cell, e.g. a neuron, an endothelial cell, a cardiomyocyte, a myogenic cell, a smooth muscle cell, or a lung cell.

In a further aspect, provided is a method for preventing or treating one or more of: vascular diseases, neuronal diseases, or a hyper-inflammation in a subject in need thereof comprising administering to a subject in need thereof an effective amount of an EMN of as disclosed herein, and/or a plurality of EMNs as disclosed herein.

The shell and/or cargo of the EMN(s) used for this treatment method as well as any other methods, EMNs, compositions, kits, or embodiments/aspects thereof, can be derived from any cell(s) or any combination of cells as described herein. In one embodiment, the shell and/or the cargo of the EMN is selected for the particular treatment, patient and/or disease. For example, one of skill in the art would select an EMN derived from a neurological cell to treat a neurological disorder. In another embodiment, the cell type from which the shell and/or cargo are/is derived may be different to the one(s) damaged in the disease. For example, an EMN comprising, or consisting essentially of, or yet further consisting of one or more of the following: (1) placental cell and/or stem cell derived lipid rafts, (2) placental cell and/or stem cell derived plasma membrane, and/or (3) placental cell and/or stem cell derived conditioned medium as a cargo, may be used to treat all diseases, including but not limited to any vascular diseases, neuronal diseases, or a hyper-inflammation as disclosed herein. In a further embodiment, any one or two or all of the following: the lipid rafts, plasma membrane and cargo, is/are derived from a placental cell.

In one embodiment, vascular diseases that can be prevented or treated are selected from the group of hind limb ischemia or cardiac ischemia. In another embodiment, the neuronal diseases are selected from the group of a neurodegenerative disease or disorder, an ischemic brain injury, stroke, a moderate or a catastrophic brain injury, a chemical neurotoxin exposure, a spinal cord injury, a traumatic brain injury, Alzheimer's disease, Parkinson's disease or a spinal cord contusion, spina bifida, myelomeningocele (MMC), multiple sclerosis, demyelination, oligodendroglia degeneration, lack of oligodendrocyte precursor cell (OPC) differentiation, or paralysis. In yet another embodiment, the hyper-inflammation is caused by a viral, bacterial, fungal or parasitic infection. In a further embodiment, the infection is a coronavirus infection, such as severe acute respiratory syndrome (SARS) coronavirus (SARS-CoV), SARS-CoV-2 causing the novel coronavirus disease-2019 (COVID-19), or Middle East respiratory syndrome (MERS) coronavirus (MERS-CoV). In yet another embodiment, the hyper-inflammation is caused by an acute respiratory distress syndrome (ARDS), a virus induced ARDS, a pneumonia, or a drug treatment, further optionally wherein the drug treatment is selected from administering an antibody or a fragment thereof, a gene therapy (such as administering an AAV viral vector or an HSV), or a cell therapy (such as an adoptive T-cell therapy, an adoptive NK-cell therapy, or an adoptive macrophage therapy, administering CAR-T cells, CAR-NK cells and/or CAR-macrophages).

In yet a further aspect, provided is a method for treating a damaged cell or preventing the cell from being damaged comprising contacting the cell with an effective amount of an EMN as disclosed herein, and/or a plurality of EMNs as disclosed herein to the damaged cell. In one embodiment, the cell is selected from neurons, endothelial cells, a cardiomyocyte, a myogenic cell, a smooth muscle cell, or lung cells. In another embodiment, the contacting is in vitro or in vivo. In one embodiment, the neuron to be treated is damaged by a neurodegenerative disease or disorder, such as an ischemic brain injury, stroke, a moderate or a catastrophic brain injury, a chemical neurotoxin exposure, a spinal cord injury, a traumatic brain injury, Alzheimer's disease, Parkinson's disease or a spinal cord contusion, spina bifida, myelomeningocele (MCC), multiple sclerosis, demyelination, oligodendroglia degeneration, lack of oligodendrocyte precursor cell (OPC) differentiation, paralysis, or a hyper-inflammation. In another embodiment, the endothelial cell is damaged in a vascular disease, an ischemia, a cardiovascular disease, hind limb ischemia, cardiac ischemia, or a hyper-inflammation. In yet another embodiment, the lung cell is damaged by a hyper-inflammation, optionally caused by an acute respiratory distress syndrome (ARDS), a virus induced ARDS, or a pneumonia. In one embodiment, the hyper-inflammation is caused by a viral, bacterial, fungal or parasitic infection, optionally a coronavirus infection. In a further embodiment, the coronavirus is selected from severe acute respiratory syndrome (SARS) coronavirus (SARS-CoV), SARS-CoV-2 causing the novel coronavirus disease-2019 (COVID-19), or Middle East respiratory syndrome (MERS) coronavirus (MERS-CoV). In one embodiment, the hyper-inflammation is optionally due to a drug treatment. In a further embodiment, the drug treatment is selected from administering an antibody or a fragment thereof, a gene therapy, or a cell therapy. In one embodiment, the gene therapy is an adeno-associated virus therapy, and the cell therapy is selected from the group of an adoptive T-cell therapy, an adoptive NK-cell therapy, or an adoptive macrophage therapy.

In certain embodiments, particularly those relating to administration, Administration can be local or systemic, as the need may be. In one embodiment, the administration is inhalation, intravenous, intrathecal, intraspinal, intrapulmonary, intranasal, epidural, oral, or intraamniotic fluid. In another embodiment, the subject is a fetus and the composition is administered to the fetus in utero. In yet another embodiment, the administration is via aerosol inhalation.

Further provided is a kit comprising an EMN as disclosed herein, a plurality of EMNs as disclosed herein, and/or a composition as disclosed herein, and optionally, reagents and instructions for use of one or more diagnostically, as a research tool or therapeutically. In one aspect, provided is a kit comprising an EMN, or a plurality, or a composition as disclosed herein, and instructions for use. In one embodiment, the instructions comprise instruction for carrying a method as disclosed herein.

Also provided is a method of producing of an EMN as disclosed herein and/or a plurality as disclosed herein. The method comprises the following: (i) optionally hypotonically lyse cells selected from the group of: a differentiated cell; a stem cell; a cancer cell; or an immune cell: neutrophils, eosinophils, basophils, mast cells, monocytes, macrophages, dendritic cells, natural killer cells, and lymphocytes (B cells and T cells); (ii) an optional mechanical homogenization; (iii) isolate or purify the lipid rafts and/or plasma membrane from the cell, optionally via one or more of centrifugation, optionally at the same or different relative centrifugal forces, optionally using serial ultracentrifugation and collecting materials at the density of lipid rafts and/or plasma membrane; and (iv) extrude the lipid rafts and/or plasma membrane with a solution comprising cargos using an extruder, optionally the extruder comprises a filter selected from an about 50 nm to 300 nm filter, optionally an about 200 nm filter, an about 150 nm filter, an about 100 nm filter, whereby generating EMNs comprising a cargo and lipid rafts and/or plasma membrane; or (v) extrude the lipid rafts and/or plasma membrane using an extruder, optionally the extruder comprises a filter selected from an about 50 nm to 300 nm filter, optionally an about 200 nm filter, an about 150 nm filter, an about 100 nm filter, centrifuge the extruded materials, remove supernatant and resuspend the rest martials comprising lipid rafts and/or plasma membrane using a solution comprising cargos, whereby EMNs were self-assembled from the extruded lipid rafts and/or plasma membrane encapsulating a cargo.

In one embodiment, the production method is scalable and/or produces a higher yield, for example, compared to the current available method isolating and/or purifying a native exosome. Additionally provided is an EMN and/or a plurality thereof produced via a method as disclosed herein.

In any embodiment and/or aspect relating to a cell, the cell may be a differentiated cell or a stem cell. In one embodiment, the cell is selected from the group of an endothelial cell, a cardiomyocyte, a myogenic cell, a smooth muscle cell, a neuron, an astrocyte, an oligodendrocyte, an olfactory ensheathing cell, a microglial cell, a tumor cell, a cancer cell, an immune cell, a neutrophil, an eosinophil, a basophil, a mast cell, a monocyte, a macrophage, a dendritic cell, a natural killer cell, a lymphocyte, a B cell or a T cell. Additionally or alternatively, the cell is an animal cell, a mammalian cell or a human cell. In certain embodiments, the stem cell is an adult stem cell and/or an embryonic stem cell. In a further embodiment, the stem cell is selected from a neuronal stem cell, an endothelial progenitor cell (EPC), a cord-blood derived EPC, a umbilical cord-derived EPCs, a mesenchymal stem cell, an adipose derived stem cell, a bone marrow derived stem cell, a placental-derived MSC (PMSC), or an induced pluripotent stem cell (iPSC). In yet a further embodiment, the mesenchymal stem cell expresses one or more of CD105⁺, CD90⁺, CD73⁺, CD44⁺ and CD29⁺ and CD184⁺. Additionally or alternatively, the mesenchymal stem cell lacks one or more of hematopoietic markers. In a further embodiment, the hematopoietic markers are selected from the group of: CD31, CD34 and CD45. In certain embodiments, the stem cell is a mesenchymal stem cell that expresses one or more exosome specific markers selected from the group of CD9, CD63, ALIZ, TSG101, alpha 4 integrin, beta 1 integrin, and/or the stem cell is a mesenchymal stem cell lacks expression of calnexin. In one embodiment, a human stem cell. In certain embodiments, the stem cell is isolated from a pediatric, fetal, early-gestation or pre-term placenta-derived stem cell. In one embodiment, the cell is an apoptotic cell. In another embodiment, the neuron is an isolated cortical neuron or a spinal cord neuron.

Other aspect and/or embodiments of the inventions will be apparent in view of the following description.

### BRIEF DESCRIPTION OF THE DRAWINGS

FIGS. 1A-1C provide a summary of the steps involved in the synthesis of EMNs. (FIG. 1A) Isolation of lipid rafts from hPMSCs. (FIG. 1B) Synthesis of nanovesicles using the Mini Extruder. (FIG. 1C) Neuroprotection assay using WimNeuron analysis.

FIGS. 2A-2C further illustrate the process of producing EMNs comprising hPMSC secretome. (FIG. 2A) procedures of concentration of conditioned media. (FIG. 2B) Lipid rafts isolation and characterization. Also listed are markers to confirm lipid rafts retain the receptors and ligands. (FIG. 2C) The assembly of the MiniExtruder™. The MiniExtruder™ consists of two gas tight syringes either side of a polycarbonate filter assembly. The bottom image represents a de-constructed image of the polycarbonate filter assembly. The polycarbonate membrane has pore sizes from 400 nm-100 nm and in replaced after each extrusion. During each extrusion, the lipid raft (for example, those comprising hPMSC secretome and/or FITC-BSA/Biotin reconstituted lipid raft) are injected from one syringe to the other through the polycarbonate filter. The mechanical pressure generated during injection allow the membranes to disassemble and reassemble thus allowing the encapsulation of the concentrated conditioned media free of exosomes and/or FITC-BSA/Biotin solution. See, for example, avantilipids.com/divisions/equipment/.

FIGS. 3A-3C show evaluation of the protein loading efficiency (which is also referred to as encapsulation efficiency) in the EMNs. (FIG. 3A) Production of EMNs comprising hPMSC lipid rafts and FITC-BSA. (FIG. 3B) Rational of measuring the cargo loading/encapsulation with a representative standard curve obtained. (FIG. 3C) Equations for calculating a loading/encapsulation efficiency of EMNs comprising FITC-BSA or hPMSC secretome. Briefly, absorbance of the supernatant at the wavelength of 525 nm was measured, reflecting the concentration of the fluorescent protein FITC. Thus, the amount of the FITC-BSA in the supernatant can be calculated, subtraction of which from the total FITC-BSA in the solution used for EMN production arrives at the FITC-BSA loaded in the nanovesicles.

FIGS. 4A-4D provide procedures and results of lipid raft isolation and characterization. (FIG. 4A) Outline of the lipid raft isolation protocol indicating the gradients and cell surface markers used for characterization (FIG. 4B) A representative image showing lipid-raft-containing solution after density gradient centrifugation. There box indicates the position of the lipid raft (seen as a white opaque ring). Percentages on the left indicate the sucrose gradient. (FIG. 4C) A representative dot blot result indicating positive signal for Caveolin-1. Percentages on top indicate the sucrose gradient. (FIG. 4D) A representative western blot result indicating the presence of lipid raft and exosome-specific markers. Double bands indicate duplicate lane containing the same sample.

FIGS. 5A-5D show determination of loading efficiency and analysis of the loaded EMNs. (FIG. 5A) Efficiency of loading FITC-BSA or FITC-Biotin as cargos. Three bars on the left indicate the loading efficiency of EMNs loaded with 0.25, 0.5 and 1 mg/mL FITC-BSA. The three bars on the right indicate EMNs loaded with 0.25, 0.5 and 1 mg/mL of FITC-Biotin. (FIG. 5B) The NTA analysis of EMNs showing the size and concentration of EMNs. (FIG. 5C) NTA image indicating the EMNs loaded with FITC-BSA. (FIG. 5D) TEM micrograph showing an EMN loaded with 0.5 mg/mL FITC-BSA. White arrows indicate EMNs.

FIGS. 6A-6E provide analyses of BSA-depleted conditioned medium. (FIG. 6A) BSA depletion using HiTrap™ BSA-column, BSA entrapped within the column. MW standards refer to molecular weight standards. The levels of BDNF (FIG. 6B), HGF (FIG. 6C), VEGF (FIG. 6D) before (left bar of each panel) and after (right bar of each panel) BSA depletion. (FIG. 6E) Levels of BDNF analyzed by ELISA of various samples. CM stored, hPMSC 48-hour conditioned medium stored for 30 days at −80° C. 48 h CM, 48-hour conditioned medium. 24 h CM, hPMSC 24-hour conditioned medium. The term “before” refers to before BSA depletion while the term “after” refers to after BSA depletion. n=1. Mean±SD across triplicates of the same sample.

FIGS. 7A-7C show neuroprotective effects of EMNs. (FIG. 7A) NTA results of EMNs loaded with concentrated conditioned medium. (FIG. 7B) TEM image of EMNs loaded with concentrated conditioned medium ranging from 50 nm-200 nm. White arrows indicate EMNs. (FIG. 7C) Neuroprotection assay showing normal SH-SY5Y (i), staurosporine-treated SH-SY5Y cells further treated with PBS only (ii), or 1000 (iii), 2000 (iv), 4000 (v), 8000 (vi) EMNs/cell. Cell morphology was compared to normal SH-SY5Y cells that were not treated with staurosporine (i). Scale bar=200 μm.

FIGS. 8A-8B show characterization of isolated plasma membrane (PM). (FIG. 8A) Western blot of cell lysate (CL) and isolated plasma membrane fraction (PM). Left: EPC (CD31) and plasma membrane (caveolin-1, calnexin (negative control)) specific markers. Right: Characteristic EV markers. (FIG. 8B) Proteomic analysis of isolated plasma membrane using tandem mass spectrophotometry. Proteins were identified using cluster analysis via Scaffold software. A total of ˜3472 proteins in 2781 clusters were identified.

FIG. 9 provides a representative miRNA release profile from miR126-loaded PLGA nanoparticles in PBS at 37° C. n=3.

FIG. 10 provides representative fluorescent microscopy images of EMNs. Arrows show one coated particle. (i) DiI-loaded PLGA core. (ii) PKH67-labeled PM. (iii) Composite image of EMNs. Scale=20 μm.

FIGS. 11A-11B show that PLGA nanoparticles, EMNs, or PM vesicles (without cargos) were dispersed in water and stored at 4° C. Size (FIG. 11A) and polydispersity index (PDI) (FIG. 11B) was measured over 28 days to observe particle size and stability. Measurements were taken in triplicate.

FIG. 12 shows a proof-of-concept study indicating that DBCO-sulfo-NHS can be used to conjugate azide-SILY onto PM. Fluorescence microscopy images of the bio-conjugation of azide-Cy5 dye to PM dyed with DiO and in the absence of DBCO (left) or presence of DBCO (right), confirming strong conjugation to the EPC PM in presence of the DBCO-sulfo-NHS. Scale=50 μm.

FIG. 13 shows binding of PLGA, EPC-EMN, and SILY-EPC-EMN to collagen under physiologically relevant peristaltic flow conditions. Scale=50 μm.

FIG. 14 provides a quantification of scratch assay. miR126-loaded PLGA nanoparticles (p<0.01, compared to PBS and empty PLGA nanoparticles) and empty EMN (p<0.05, compared to PBS control) can promote endothelial progenitor cell (EPC) migration. n=3.

FIGS. 15A-15B show that PM coating improves particle uptake by endothelial progenitor cells (EPCs). PLGA nanoparticles (NPs) (FIG. 15A) or EPC EMNs (FIG. 15B) were added to EPCs and incubated for 24 hours. Cells were fixed and stained for the nucleus (DAPI) and surface marker CD31. Internalized particles are visualized (DIO-loaded PLGA). Scale bar=100 μm. n=3.

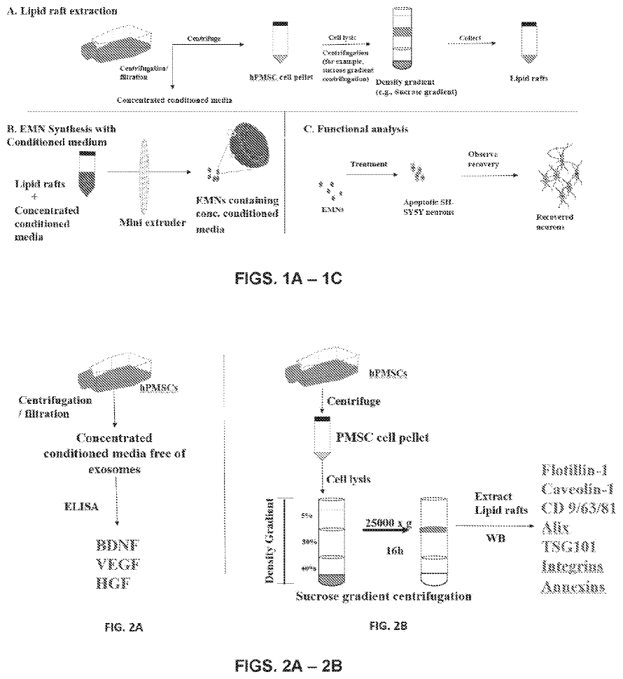

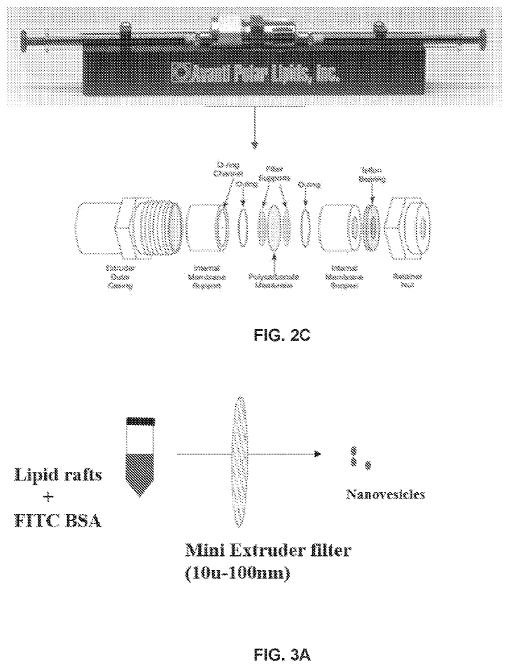

### DETAILED DESCRIPTION

### Definitions

The practice of the present invention will employ, unless otherwise indicated, conventional techniques of tissue culture, immunology, molecular biology, microbiology, cell biology and recombinant DNA, which are within the skill of the art. See, e.g., Sambrook, Fritsch and Maniatis (1989) Molecular Cloning: A Laboratory Manual, 2^{nd} edition; F. M. Ausubel, et al. eds. (1987) Current Protocols In Molecular Biology; the series Methods in Enzymology (Academic Press, Inc.): PCR 2: A Practical Approach (1995) (M. J. MacPherson, B. D. Hames and G. R. Taylor eds.); Harlow and Lane, eds. (1988) Antibodies, A Laboratory Manual; Harlow and Lane, eds. (1999) Using Antibodies, a Laboratory Manual; and R. I. Freshney, ed. (1987) Animal Cell Culture.

All numerical designations, e.g., pH, temperature, time, concentration, and molecular weight, including ranges, are approximations which are varied (+) or (−) by increments of 1.0 or 0.1, as appropriate. It is to be understood, although not always explicitly stated that all numerical designations are preceded by the term “about”. It also is to be understood, although not always explicitly stated, that the reagents described herein are merely exemplary and that equivalents of such are known in the art.

The term “about” as used herein when referring to a measurable value such as an amount or concentration and the like, is meant to encompass variations of 20%, 10%, 5%, 1%, 0.5%, or even 0.1% of the specified amount.

As used in the specification and claims, the singular form “a,” “an” and “the” include plural references unless the context clearly dictates otherwise.

As used herein, the term “comprising” is intended to mean that the compositions and methods include the recited elements, but do not exclude others. “Consisting essentially of” when used to define compositions and methods, shall mean excluding other elements of any essential significance to the combination when used for the intended purpose. Thus, a composition consisting essentially of the elements as defined herein would not exclude trace contaminants or inert carriers. “Consisting of” shall mean excluding more than trace elements of other ingredients and substantial method steps. Embodiments defined by each of these transition terms are within the scope of this invention.

used herein, the terms “increased”, “decreased”, “high”, “low” or any grammatical variation thereof refer to a variation of about 90%, 80%, 50%, 20%, 10%, 5%, 1%, 0.5%, or even 0.1% of the reference.

The terms or “acceptable,” “effective,” or “sufficient” when used to describe the selection of any components, ranges, dose forms, etc. disclosed herein intend that said component, range, dose form, etc. is suitable for the disclosed purpose.

The term “isolated” as used herein refers to molecules or biological or cellular materials being substantially free from other materials, e.g., greater than 70%, or 80%, or 85%, or 90%, or 95%, or 98%. In one aspect, the term “isolated” refers to nucleic acid, such as DNA or RNA, or protein or polypeptide, or lipid rafts or plasma membrane, or cell or cellular organelle, or tissue or organ, separated from other DNAs or RNAs, or proteins or polypeptides, or cells or cellular organelles, or tissues or organs, respectively, that are present in the natural source and which allow the manipulation of the material to achieve results not achievable where present in its native or natural state, e.g., recombinant replication or manipulation by mutation. The term “isolated” also refers to one or more of the following: a nucleic acid, a peptide, a protein, a lipid raft, and/or a plasma membrane, that is substantially free of other cellular material, viral material, or culture medium when produced by recombinant DNA techniques, or chemical precursors or other chemicals when chemically synthesized, or concentration and/or purification techniques, e.g., with a purity greater than 0.1%, or 1%, or 2%, or 3%, or 10%, or 20%, or 30%, or 40%, or 50%, or 60%, 70%, or 80%, or 85%, or 90%, or 95%, or 98%. In one embodiment, such purity percentage may refer to a weight or volume ratio of the isolated materials to the total composition (for example, a solution). In another embodiment, the purity percentage refer to gram of the isolated materials per 100 mL of the total composition (for example, a solution). Moreover, an “isolated nucleic acid” is meant to include nucleic acid fragments which are not naturally occurring as fragments and would not be found in the natural state. The term “isolated” is also used herein to refer to polypeptides which are isolated from other cellular proteins and is meant to encompass both purified and recombinant polypeptides, e.g., with a purity greater than 70%, or 80%, or 85%, or 90%, or 95%, or 98%. The term “isolated” is also used herein to refer to cells or tissues that are isolated from other cells or tissues and can encompass cultured and/or engineered cells or tissues.

As used herein the terms “purification”, “purifying”, or “separating” refer to the process of isolating one or more component from a complex mixture, such as a cell lysate or a mixture of polypeptides. Non-limiting examples of the component include nucleic acid, such as DNA or RNA, or protein or polypeptide, or lipid rafts or plasma membrane, or cell or cellular organelle, or tissue or organ, separated from other DNAs or RNAs, or proteins or polypeptides, or cells or cellular organelles, or tissues or organs, The purification, separation, or isolation need not be complete, i.e., some other components of the complex mixture may remain after the purification process. However, the product of purification should be enriched for the component relative to the complex mixture before purification and a significant portion of the other components initially present within the complex mixture should be removed by the purification process.

The term “cell” as used herein may refer to either a prokaryotic or eukaryotic cell, optionally obtained from a subject or a commercially available source. In one embodiment the cell here is capable of producing an exosome naturally. In a further embodiment the cell here is a stem cell. Additionally or alternatively, dysfunction of the cell may lead to a disorder.

“Eukaryotic cells” comprise all of the life kingdoms except monera. They can be easily distinguished through a membrane-bound nucleus. Animals, plants, fungi, and protists are eukaryotes or organisms whose cells are organized into complex structures by internal membranes and a cytoskeleton. The most characteristic membrane-bound structure is the nucleus. Unless specifically recited, the term “host” includes a eukaryotic host, including, for example, yeast, higher plant, insect and mammalian cells. Non-limiting examples of eukaryotic cells or hosts include simian, bovine, porcine, murine, rat, avian, reptilian and human.

As used herein, the term “animal” refers to living multi-cellular vertebrate organisms, a category that includes, for example, mammals and birds. The term “mammal” includes both human and non-human mammals.

“Prokaryotic cells” that usually lack a nucleus or any other membrane-bound organelles and are divided into two domains, bacteria and archaea. In addition to chromosomal DNA, these cells can also contain genetic information in a circular loop called an episome. Bacterial cells are very small, roughly the size of an animal mitochondrion (about 1-2 μm in diameter and 10 μm long). Prokaryotic cells feature three major shapes: rod shaped, spherical, and spiral. Instead of going through elaborate replication processes like eukaryotes, bacterial cells divide by binary fission. Examples include but are not limited to Bacillus bacteria, E. coli bacterium, and Salmonella bacterium. As used herein, “stem cell” defines a cell with the ability to divide for indefinite periods (i.e., self-renewal) in a subject and/or in culture and give rise to specialized cells (i.e., differentiation). At this time and for convenience, stem cells are categorized as somatic (adult), embryonic or induced pluripotent stem cells. A somatic stem cell is an undifferentiated cell found in a differentiated tissue that can renew itself (clonal) and (with certain limitations) differentiate to yield all the specialized cell types of the tissue from which it originated. An embryonic stem cell is a primitive (undifferentiated) cell from the embryo that has the potential to become a wide variety of specialized cell types. Non-limiting examples of embryonic stem cells are the HES2 (also known as ES02) cell line available from ESI, Singapore and the H1 or H9 (also known as WA01) cell line available from WiCell, Madison, Wis. Pluripotent embryonic stem cells can be distinguished from other types of cells by the use of markers including, but not limited to, Oct-4, alkaline phosphatase, CD30, TDGF-1, GCTM-2, Genesis, Germ cell nuclear factor, SSEA1, SSEA3, and SSEA4. An -induced pluripotent stem cell (iPSC) is an artificially derived stem cell from a non-pluripotent cell, typically an adult somatic cell, produced by inducing expression of one or more stem cell specific genes. In one embodiment, the stem cell may refer to a “parthenogenetic stem cell” which is a stem cell arising from parthenogenetic activation of an egg. Methods of creating a parthenogenetic stem cell are known in the art. See, for example, Cibelli et al. et al. (2002) Science 295(5556):819 and Vrana et al. et al. (2003) Proc. Natl. Acad. Sci. USA 100(Suppl. 1)11911-6 (2003).

“Embryoid bodies or EBs” are three-dimensional (3-D) aggregates of embryonic stem cells formed during culture that facilitate subsequent differentiation. When grown in suspension culture, EBs cells form small aggregates of cells surrounded by an outer layer of visceral endoderm. Upon growth and differentiation, EBs develop into cystic embryoid bodies with fluid-filled cavities and an inner layer of ectoderm-like cells.

The term “propagate” means to grow or alter the phenotype of a cell or population of cells. The term “growing” refers to the proliferation of cells in the presence of supporting media, nutrients, growth factors, support cells, or any chemical or biological compound necessary for obtaining the desired number of cells or cell type. In one embodiment, the growing of cells results in the regeneration of tissue. In yet another embodiment, the tissue is comprised of neuronal progenitor cells or neuronal cells.

The term “culturing” refers to the in vitro propagation of cells or organisms on or in media of various kinds. It is understood that the descendants of a cell grown in culture may not be completely identical (i.e., morphologically, genetically, or phenotypically) to the parent cell. By “expanded” is meant any proliferation or division of cells. A “cultured” cell is a cell that has been separated from its native environment and propagated under specific, pre-defined conditions. Such culture may be performed in a bioreactor supporting a biologically active environment (e.g., temperature, O₂% and CO₂%). In one embodiment, the bioreactor is a closed and/or continuous bioreactor. Additionally or alternatively, the bioreactor is a three dimensional bioreactor.

“Differentiation” describes the process whereby an unspecialized cell acquires the features of a specialized cell such as a heart, liver, or muscle cell. “Directed differentiation” refers to the manipulation of stem cell culture conditions to induce differentiation into a particular cell type. “Dedifferentiated” defines a cell that reverts to a less committed position within the lineage of a cell. As used herein, the term “differentiates or differentiated” defines a cell that takes on a more committed (“differentiated”) position within the lineage of a cell. As used herein, “a cell that differentiates into a mesodermal (or ectodermal or endodermal) lineage” defines a cell that becomes committed to a specific mesodermal, ectodermal or endodermal lineage, respectively. Examples of cells that differentiate into a mesodermal lineage or give rise to specific mesodermal cells include, but are not limited to, cells that are adipogenic, leiomyogenic, chondrogenic, cardiogenic, dermatogenic, hematopoetic, hemangiogenic, myogenic, nephrogenic, urogenitogenic, osteogenic, pericardiogenic, or stromal.

Examples of cells that differentiate into ectodermal lineage include, but are not limited to epidermal cells, neurogenic cells, and neurogliagenic cells.

A “marrow stromal cell” are used interchangeably with “mesenchymal stem cells,” or MSC, is a multipotent stem cell that can differentiate into a variety of cell types. Cell types that MSCs have been shown to differentiate into in vitro or in vivo include osteoblasts, chondrocytes, myocytes, adipocytes, endothelial cells, odontoblasts and neurons. Mesenchyme is embryonic connective tissue that is derived from the mesoderm and that differentiates into hematopoietic and connective tissue, whereas MSCs do not differentiate into hematopoietic cells. Stromal cells are connective tissue cells that form the supportive structure in which the functional cells of the tissue reside. Methods to isolate such cells, propagate and differentiate such cells are known in the technical and patent literature, e.g., U.S. Patent Application Publication Nos. 2007/0224171, 2007/0054399 and 2009/0010895, which are incorporated by reference in their entirety.

“pMSC” or “PMSC” or “mpSCs” are acronyms for mesenchymal stem cells isolated or purified from placental tissue prior to delivery of the fetus by surgery or birth. Within this disclosure, the cells also are referred to as pre-term placenta-derived stem cell (mpSCs) or when isolated by chorionic villus sampling, they are identified as C-mpSCs. In one aspect, the PMSC express angiogenic and immunomodulatory cytokines (e.g. Angiogenin, Angiopoietin-1, HGF, VEGF, IL-8, MCP-1, uPA).

Chorionic Villus Sampling (CVS) is a technique to diagnose complications during a pregnancy. A small section of placental tissue is collected without disturbing the pregnancy, and these cells are examined for disease. The tissue sampled is the chorionic villus, and CVS is the technique used to obtain chorionic villus samples.

The term “encode” as it is applied to nucleic acid sequences refers to a polynucleotide which is said to “encode” a polypeptide if, in its native state or when manipulated by methods well known to those skilled in the art, can be transcribed and/or translated to produce the mRNA for the polypeptide and/or a fragment thereof. The antisense strand is the complement of such a nucleic acid, and the encoding sequence can be deduced therefrom.

As used herein, “expression” refers to the process by which polynucleotides are transcribed into mRNA and/or the process by which the transcribed mRNA is subsequently being translated into peptides, polypeptides, or proteins. If the polynucleotide is derived from genomic DNA, expression may include splicing of the mRNA in a eukaryotic cell.

As used herein, the terms “nucleic acid sequence” and “polynucleotide” are used interchangeably to refer to a polymeric form of nucleotides of any length, either ribonucleotides or deoxyribonucleotides. Thus, this term includes, but is not limited to, single-, double-, or multi-stranded DNA or RNA, genomic DNA, complementary DNA (cDNA), DNA-RNA hybrids, or a polymer comprising purine and pyrimidine bases or other natural, chemically or biochemically modified, non-natural, or derivatized nucleotide bases. In certain embodiments, the polynucleotide comprises and/or encodes a messenger RNA (mRNA), a short hairpin RNA, and/or small hairpin RNA. In one embodiment, the polynucleotide is or encodes an mRNA. In certain embodiments, the polynucleotide is a double-strand (ds) DNA, such as an engineered ds DNA or a ds cDNA synthesized from a single-stranded RNA. A polynucleotide disclosed herein can be delivered to a cell or tissue using an EMN as described herein. As used herein, when referring to the length of a polynucleotide, the unit “nucleotides” i.e., “nt” is used. In the embodiments of a single-strand polynucleotide, the length of the polynucleotide is presented herein as the total number of nucleotide residues that the polynucleotide comprises. In the embodiments of a double-strand or multi-strand polynucleotide, the length of the polynucleotide is presented as the number of the total number of nucleotide residues that the longest change of the polynucleotide comprises.

As used herein, the terms “engineered” “synthetic” “recombinant” and “non-naturally occurring” are interchangeable and indicate intentional human manipulation, for example, a modification from its naturally occurring form, and/or a sequence optimization.

As used herein, N/P ratio, or basically the ratio of positively-chargeable polymer amine (N=nitrogen) groups to negatively-charged nucleic acid phosphate (P) groups, is one of the most important physicochemical properties of polymer-based gene delivery vehicles which is also adopted herein for quantifying the physiochemical properties of the cargo-core complex and/or an EMNs as described herein. The N/P character of a polymer/nucleic acid complex can influence many other properties such as its net surface charge, size, and stability. At high N/P ratios, especially ones well above the point required to form charge-neutralized complexes with siRNA, important questions arise about how these complexes will behave in an in vivo environment in response to the excess cationic charge. One important implication of N/P ratio in DNA polyplex systems is the enhancement in in vitro gene expression that is typically observed at high N/P ratios as a result of free cationic polymer which enhances intracellular delivery.

The term “protein”, “peptide” and “polypeptide” are used interchangeably and in their broadest sense to refer to a compound of two or more subunits of amino acids, amino acid analogs or peptidomimetics. The subunits may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence. As used herein the term “amino acid” refers to either natural and/or unnatural or synthetic amino acids, including glycine and both the D and L optical isomers, amino acid analogs and peptidomimetics.

As used herein, the terms “conjugate,” “conjugated,” “conjugating,” and “conjugation” refer to the formation of a bond between molecules, and in particular between two amino acid sequences and/or two polypeptides. Conjugation can be direct (i.e. a bond) or indirect (i.e. via a further molecule). The conjugation can be covalent or non-covalent.

An “effective amount” or “efficacious amount” refers to the amount of an agent (such as an EMN as disclosed herein), or combined amounts of two or more agents, that, when administered for the treatment of a subject, is sufficient to effect such treatment for the disease. The “effective amount” will vary depending on the agent(s), the disease and its severity and the age, weight, etc., of the subject to be treated.

The terms “subject,” “host,” “individual,” and “patient” are as used interchangeably herein to refer to human and veterinary subjects, for example, humans, animals, non-human primates, dogs, cats, sheep, mice, horses, and cows. In some embodiments, the subject is a human.

A “pharmaceutical composition” is intended to include the combination of an agent (such as an EMN as disclosed herein) with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

The term “tissue” is used herein to refer to tissue of a living or deceased organism or any tissue derived from or designed to mimic a living or deceased organism. The tissue may be healthy, diseased, and/or have genetic mutations. The biological tissue may include any single tissue (e.g., a collection of cells that may be interconnected) or a group of tissues making up an organ or part or region of the body of an organism. The tissue may comprise a homogeneous cellular material or it may be a composite structure such as that found in regions of the body including the thorax which for instance can include lung tissue, skeletal tissue, and/or muscle tissue. Exemplary tissues include, but are not limited to those derived from liver, lung, thyroid, skin, pancreas, blood vessels, bladder, kidneys, brain, biliary tree, duodenum, abdominal aorta, iliac vein, heart and intestines, including any combination thereof.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, the term “treatment” excludes prevention.

As used herein, the phrase “rescuing a cell” or any grammatical variation thereof refers to one of more of the following: (1) improving the cell viability, such as making the cell alive for a long time period; (2) enhancing a cell function; (3) making the cell show a morphology of a healthy control cell; (4) making the cell not show a damaged cell morphology; (5) bringing the cell morphology closer to that of a healthy control cell and/or less like a damaged cell morphology; (6) making the cell have an expression profile of a healthy control cell; (7) making the cell not have an expression profile of a damaged cell; or (8) making the cell expression profile closer to that of a healthy control cell and/or less like a damaged cell. In one embodiment, rescuing a cell refers to preventing, delaying, inhibiting, ameliorating or reversing one or more of the following in a cell: cell death, apoptosis, necrosis, and/or lose of a cell function.

As used herein, the term “cell morphology” or any variation thereof refers to an important aspect of the phenotype of a cell, including the shape, structure, form, and size of cells. Neuron cell morphology is further described and measure in the Examples.

Expression profile is another aspect of the phenotype of a cell. As used herein, it refers to one or more or part of or all molecule as well as presence and/or abundance in a cell. Such cell molecule include but not limited to a polynucleotide (such as mRNA), a polypeptide or protein, or a lipid. In one embodiment, the expression profile comprise presence or level of an apoptotic marker in a cell. See e.g., abcam.com/kits/apoptosis-assays for available apoptotic markers and assays.

As used herein, a “damaged cell” refers to one or more of the cell morphology, expression profile cell function and/or cell viability are less desirable compared to a healthy control.

Membranous vesicles secreted by cells are collectively termed extracellular vesicles (EVs), of which there are three main subtypes: exosomes, microvesicles and apoptotic bodies. Exosomes are the smallest type of EVs (50-150 nm in diameter) and are released following the fusion of late endosomes and multi-vesicular bodies within the plasma membrane. Exosomes are naturally occurring nanosized vesicles and comprised of natural lipid bilayers with the abundance of adhesive proteins that readily interact with cellular membranes. These vesicles have a content that includes cytokines and growth factors, signaling lipids, mRNAs, and regulatory miRNAs. In multicellular organisms, exosomes and other EVs are present in tissues and can also be found in biological fluids including blood, urine, and cerebrospinal fluid. They are also released in vitro by cultured cells into their growth medium.

Referred to herein as a native exosome is an exosome that is naturally occurring, released from a cell without human intervention and optionally purified. Certain native exosome-specific markers are well known in the art, such as integrin α4β1, CD 81, CD 9 and CD 63. A nanovesicle sharing the same morphology (such as size) and function of a native exosome but are produced with human intervention (such as using the method as described in the Example) is referred to herein as an exosome mimicking nanovesicle (EMN). Both native exosomes and EMNs comprises a “shell” (whose composition is similar to a plasma membrane) forming the vesicle wall/barrier (i.e., “shell”) and encapsulating certain molecules as content within the vesicle. In certain embodiments, an exogenous agent/molecule (such as a polynucleotide, a peptide/protein, or a small molecular, optionally heterologous to the subject/tissue/cell from which the shell is derived) encapsulated within an EMN shell is referred to herein as a cargo. Without wishing to be bound by the theory, such cargo molecular may be further loaded (conjugated or unconjugated linked) to a core which facilitates loading the cargo to a vesicle, improve the cargo's stability, and/or provide a sustained release of cargo(s) free of the core. Such core is normally inert and does not perform any other biological function in the EMN, after being released from the EMN, in a cell culture and/or in a subject. In certain embodiments, a cargo comprises a core as described herein.

Lipid rafts are highly organized plasma membrane microdomains enriched in phospholipids, glycosphingolipids, and cholesterol, and serve as matrix for receptors, such as G protein coupled receptors (GPCRs), and other signaling molecules. See, for example, Villar et al. (2016). Lipid rafts are subdomains of plasma membrane (10-200 nm) rich in glycosphingolipids and cholesterol that play an active role in signal transduction. Rafts appear to be small in size, but may constitute a relatively large fraction of the plasma membrane. The lipid rafts are of two main types, planar lipid rafts and caveolae. Planar lipid rafts are continuous with the plasma membrane and contain flotillin proteins, while caveolae are inviganitated lipid rafts and composed of caveolin 1 proteins. It is noted that during the biosynthesis of exosomes and as the exosomes proceed through the different stages, they retain the raft proteins. In most cases, raft structure is relatively stable and resemble the composition of the cell membrane

As used herein, cell derived conditioned medium refers to a culture medium collected after culturing cells for a certain time period, such as 24 hours or 48 hours, and containing molecules (such as polynucleotide, or peptide/protein) and other components (such as certain vesicles) secreted by the cultured cell into the extracellular space. In one embodiment, such conditioned medium is substantially free of any cell. Additionally or alternatively, the conditioned medium is substantially free of any native exosomes. In a further embodiment, the conditioned medium is further purified and/or condensed so that the concentration of one or more of the molecules in the medium is increased. In yet a further embodiment, one or more of undesired molecules may be removed from the medium. In one embodiment, the conditioned medium comprises a cell secretome which is the set of proteins expressed by an organism and secreted into the extracellular space, for example a secretome of hPMSC.

As used herein, the terms “antibody,” “antibodies” and “immunoglobulin” includes whole antibodies and any antigen binding fragment or a single chain thereof. Thus the term “antibody” includes any protein or peptide containing molecule that comprises at least a portion of an immunoglobulin molecule. The terms “antibody,” “antibodies” and “immunoglobulin” also include immunoglobulins of any isotype, fragments of antibodies which retain specific binding to antigen, including, but not limited to, Fab, Fab′, F(ab)2, Fv, scFv, dsFv, Fd fragments, dAb, VH, VL, VhH, and V-NAR domains; minibodies, diabodies, triabodies, tetrabodies and kappa bodies; multispecific antibody fragments formed from antibody fragments and one or more isolated. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, at least one portion of a binding protein, chimeric antibodies, humanized antibodies, single-chain antibodies, and fusion proteins comprising an antigen-binding portion of an antibody and a non-antibody protein. The variable regions of the heavy and light chains of the immunoglobulin molecule contain a binding domain that interacts with an antigen. The constant regions of the antibodies (Abs) may mediate the binding of the immunoglobulin to host tissues. The antibodies can be polyclonal, monoclonal, multispecific (e.g., bispecific antibodies), and antibody fragments, so long as they exhibit the desired biological activity. Antibodies can be isolated from any suitable biological source, e.g., murine, rat, sheep and canine.

Within the fields of molecular biology and pharmacology, a small molecule is a low molecular weight (<900 daltons) organic compound that may regulate a biological process, with a size on the order of 1 nm. Many drugs are small molecules, such as Gefitinib, Erlotinib, Sunitinib, Bortezomib, Batimastat, Obatoclax and Navitoclax. Larger structures such as nucleic acids and proteins, and many polysaccharides are not small molecules, although their constituent monomers (ribo- or deoxyribonucleotides, amino acids, and monosaccharides, respectively) are often considered small molecules. Small molecules may be used as research tools to probe biological function as well as leads in the development of new therapeutic agents. In one embodiment, a small molecular is used interchangeably with small molecular drug that can enter cells easily because it has a low molecular weight. Once inside the cells, it can affect other molecules, such as proteins, and may cause cancer cells to die. This is different from drugs that have a large molecular weight, which keeps them from getting inside cells easily. In one embodiment, the small molecule refers to a chemical compound which is composed of many identical molecules (or molecular entities) composed of atoms from more than one element held together by chemical bonds. In another embodiment, the small molecule refers to a biological molecule which can be produced by cells and/or living organisms in a low molecular weight.

A therapeutic agent, as used herein refer to any chemical compound, biological molecule (e.g. polynucleotide, vector, polypeptide/protein, lipid, carbohydrate), cellular organelle, cell, modified cells (such as CAR-T cell, CAR-NK cell, CAR-macrophages), cell population, tissue, organ, or a pharmaceutical composition thereof, which exhibit certain biological functions (such as treating a disease, treating a damaged cell and/or rescuing a cell).

As used herein, cell-penetrating peptides (CPPs) are short peptides that facilitate cellular intake/uptake of various molecular equipment (from nanosize particles to small chemical molecules and large fragments of DNA, such as a native exosome and/or an EMN as disclosed herein). The function of the CPPs are to deliver the cargo into cells, for example via a process that commonly occurs through endocytosis. CPPs typically have an amino acid composition that either contains a high relative abundance of positively charged amino acids such as lysine or arginine or has sequences that contain an alternating pattern of polar/charged amino acids and non-polar, hydrophobic amino acids. These two types of structures are referred to as polycationic or amphipathic, respectively. A third class of CPPs are the hydrophobic peptides, containing only apolar residues, with low net charge or have hydrophobic amino acid groups that are crucial for cellular uptake. Non-limiting examples of CPPs include BP100, 2BP100, Rev(34-50), R9, D-R9, R12, KH9, K9, K18, Pen2W2F, DPV3, 6-Oct, R9-TAT, Tat(49-57), Retro-Tat(57-49), Sc18, KLA10, IX, XI, No. 14-12, pVEC, PenArg, M918, and Penetratin. See, for example, www.lifetein.com/Cell_Penetrating_Peptides.html for more CPPs as well as their amino acid sequences.

Cell targeting peptides (CTPs) are small peptides which have high affinity and specificity to a cell or tissue targets. They are typically identified by using phage display and chemical synthetic peptide library methods. Suitable CTPs can be readily selected by one of skill in the art, for example, a peptide having a sequence of QPWLEQAYYSTF (SEQ ID NO: 2) may be used to target a normal endothelium while a peptide having a sequence of YPHIDSLGHWRR (SEQ ID NO: 3) may be used to target a hypoxic endothelium. See, Andrieu et al. (2019).

HLA-G histocompatibility antigen, class I, G, also known as human leukocyte antigen G (HLA-G), is a protein that in humans is encoded by the HLA-G gene. As a non-classical major histocompatibility complex (MHC) class I molecule, HLA-G inhibits natural killer cell (NK) killing. Unlike bone marrow-derived MSCs (BM-MSCs), Placenta-derived MSCs express HLA-G on their surface in response to interferon gamma (IFNγ), which is a key inflammatory mediator involved with the onset of multiple sclerosis (MS). Therefore, the expression of HLA-G on PMSCs would make them a unique therapeutic cell source for the treatment of neurodegenerative diseases like MS. Without wishing to be bound by the theory, presence of HLA-G in an EMN as disclosed herein may also slow clearance of the EMNs in a subject via immune responses, thus improving the effectiveness of the EMN treatment.

As used herein, the term “scaffold” refers to a substrate (such as implants or injects) suitable for loading and/or delivering an EMN as disclosed herein into a subject. In one embodiment, a suitable scaffold may serve a function other than delivering the EMN, such as a stent which is a tubular support placed temporarily inside a blood vessel, canal, or duct to aid healing or relieve an obstruction, or a graft which is healthy skin, bone, kidney, liver, or other tissue that is taken from one part of the body or one subject to replace diseased or injured tissue removed from another part of the body or another subject, respectively. In another embodiment, the scaffold is selected from a medical material or a medical device which is suitable for delivering to a subject. Biocompatible matrix refers to a substrate suitable for such deliver too while the substrate is biocompatible, i.e., not harmful to living cell/tissue/subject.

The term “implant” refers to a device manufactured to replace a missing biological structure, support a damaged biological structure, or enhance an existing biological structure. Medical implants are man-made devices, in contrast to a “transplant”, which is a transplanted biomedical tissue. Depending on what is the most functional, various biomedical materials (such as titanium, silicone, or apatite) may be used as the implant surface that contact the body of a subject. In some embodiments, implants contain electronics, for example, artificial pacemaker and cochlear implants. Some implants are bioactive, such as subcutaneous drug delivery devices in the form of implantable pills or drug-eluting stents.

As used herein, the term “administration” or any grammatical variation thereof refers to the process of delivering an agent, for example to a subject. In certain embodiments, the administration is performed in vitro and/or ex vivo. In a further embodiment, the administration refers to an in vitro administration, such as a contacting the agent to be administered with a cell and/or cell culture. Administration can be local or systemic, as the need may be. In one embodiment, the administration is inhalation, intravenous, intrathecal, intraspinal, intrapulmonary, intranasal, epidural, oral (such as a tablet, capsule or suspension), or intraamniotic fluid. In another embodiment, the subject is a fetus and the composition is administered to the fetus in utero. In yet another embodiment, the administration is via aerosol inhalation. In one embodiment, other suitable administration route may be utilized, for example, but not limited to, topical, transdermal, vaginal, rectal, subcutaneous, intraarterial, intramuscular, intraosseous, intraperitoneal, intraocular, subconjunctival, sub-Tenon's, intravitreal, retrobulbar, intracameral, or intratumoral.

As used herein, the phase A-derived B indicates A as the source of B. In one embodiment, A is a cell. In one embodiment, A is the only source of B. In another embodiment, A is one of many sources of B. Additionally or alternatively, B is an agent, molecule and/or component, such as an exosome, an EMN, or conditioned medium. In one embodiment, “derived” refer to a process of isolation, purification and/or concentration. Additionally or alternatively, “derived” may comprises a physical process, a chemical change/modification as well as a biological reaction. As shown in the Example, whole EPCs are mechanically extruded to break the cell and the created plasm membrane self-assembled to EMNs retaining cell surface marker.

“BDNF” is an acronym for Brain Derived Neurotropic Factor that is vital to healing in the nervous system. An exemplary sequence for human BDNF protein is disclosed at Accession No.: NP_00137277 and mRNA is disclosed at NM_001143805. An exemplary murine BDNF is disclosed at NP_001041604 and mRNA is disclosed at NM_001048139.

CD56 is also known as N-CAM (neural cell adhesion molecule) and is reported to act as a hemophilic binding glycoprotein with a role in cell-cell adhesion. The human protein sequence is disclosed at P13591 (niProtKB/Swiss-Prot). A polynucleotide and protein encoded by the polynucleotide are at GenBank number NM_001076682. Additional information regarding the gene and transcripts is disclosed at genecards.org/cgi-bin/carddisp.pl?gene=NCAM1 (last accessed on Aug. 13, 2014). Antibodies to the marker and polynucleotides encoding the marker are commercially available from Sino Biological (old.sinobiological.com/NCAM1-CD56-a-6632. html, last access on Aug. 13, 2014) and Life Technologies.

CD271 is also known as the Nerve Growth Factor Receptor (NGFR). The protein is reported to contain an extracellular domain containing four 40-amino acid repeats with cysteine residues at conserved positions followed by a serine/threonine-rich region, a single transmembrane domain and a 155 amino acid cytoplasmic domain. The human protein sequence is disclosed at TNR16 HUMAN, P08138 (uniprot.org/uniprot/P08138#section_comments, last accessed on Aug. 13, 2014). A polynucleotides encoding the marker is under GenBank No. NM_002507 (see also genecards.org/cgi-bin/carddisp.pl?gene=NGFR, last accessed on Aug. 13, 2014). Antibodies are commercially available from Miltenyi Biotech and other vendors.

CD105 is also known as Endoglin (ENG) is reported to be a 658 amino acid sequence and a homodimer that forms a heteromeric complex with the signaling receptors for transforming growth factor-beta (TGFBR). A polynucleotide encoding the marker and an amino acid sequence is disclosed under GenBank No. M_001278138 (see also genecards.org/cgi-bin/carddisp.pl?gene=ENG, last accessed on Aug. 13, 2014). Antibodies to the marker are commercially available from numerous vendors, e.g., R&D Systems Antibodies, Novus Biologicals and Abcam antibodies.

CD90 also is known as Thy-1. A polynucleotide encoding the marker and an amino acid sequence are disclosed under GenBank number NM_006288. Additional information regarding the marker and vendors that provide antibodies to the marker are disclosed under Genecards reference: genecards.org/cgi-bin/carddisp.pl?gene=THY1, last accessed on Aug. 13, 2014.

CD73 also is known as NTSE. The protein is reported to be a gene is a plasma membrane protein that catalyzes the conversion of extracellular nucleotides to membrane-permeable nucleosides. The encoded protein is used as a determinant of lymphocyte differentiation. Defects in this gene can lead to the calcification of joints and arteries. Two transcript variants encoding different isoforms have been reported for this gene. See genecards.org/cgi-bin/carddisp.pl?gene=NTSE, last accessed on Aug. 13, 2014. A polynucleotides encoding the protein and an encoded amino acid sequences are disclosed under GenBank number BC065937. Antibodies to the marker are commercially available from several vendors, e.g., R&D Systems Antibodies.

CD44 is reported to be a cell-surface glycoprotein involved in cell-cell interactions, cell adhesion and migration. It is a receptor for hyaluronic acid (HA) and can also interact with other ligands, such as osteopontin, collagens, and matrix metalloproteinases (MMPs). A polynucleotide and encoded amino acid sequence are disclosed under GenBank number FJ216964 (last accessed Aug. 13, 2014). Additional information regarding the marker and commercially available antibodies to the marker are disclosed at genecards.org/cgi-bin/carddisp.pl?gene=CD44, last accessed on Aug. 13, 2014.

CD29 also is known as Integrin Beta 1, Fibronectin Receptor, Beta Polypeptide (see Genecards: genecards.org/cgi-bin/carddisp.pl?gene=ITGB1, last accessed on Aug. 13, 2014). A polynucleotide and protein encoded by the polynucleotide are disclosed under GenBank number NG_029012. Additional information regarding the marker and commercially available antibodies to the marker are disclosed at genecards.org/cgi-bin/carddisp.pl?gene=CD44, last accessed on Aug. 13, 2014.

CD184 also is known as “chemokine (C-X-C Motif) receptor.” The protein is reported to have transmembrane regions and is located on the cell surface. It acts with the CD4 protein to support HIV entry into cells and is also highly expressed in breast cancer cells. A polynucleotide and encoded amino acid sequence are disclosed under GenBank number NM_003467. Additional information regarding the marker and commercially available antibodies to the marker are disclosed at genecards.org/cgi-bin/carddisp.pl?gene=CXCR4, last accessed on Aug. 13, 2014.

CD49d (also known as ITGA4) is an integrin alpha subunit. It makes up half of the α4β1 lymphocyte homing receptor. The product of this gene is reported to be a member of the integrin alpha chain family of proteins. Integrins are heterodimeric integral membrane proteins composed of an alpha chain and a beta chain. The gene encoding CD49d encodes an alpha 4 chain. A polynucleotide and amino acid sequence encoded by it is reported under GenBank number NM_000885. Additional information regarding the marker and commercially available antibodies to the marker are disclosed at genecards.org/cgi-bin/carddisp.pl?gene=TRIM49D1, last accessed on Aug. 13, 2014.

CD49f is also known as integrin, alpha 6 or ITGA6. The product of this gene is reported to be a member of the integrin alpha chain family of proteins. A polynucleotide and amino acid sequence encoded by it is reported under GenBank number NM_000210. Additional information regarding the marker and commercially available antibodies to the marker are disclosed at genecards.org/cgi-bin/carddisp.pl?gene=ITGA6, last accessed on Aug. 13, 2014.

CD31 also is known as platelet/endothelial cell adhesion molecule 1 (PECAM1). A polynucleotide and amino acid sequence encoded by it is reported under GenBank number AF281301. Additional information regarding the marker and commercially available antibodies to the marker are disclosed at genecards.org/cgi-bin/carddisp.pl?gene=PECAM1, last accessed on Aug. 13, 2014.

CD34 is a cell surface marker. A polynucleotide and amino acid sequence encoded by it is reported under GenBank number M81104 (X60172). Additional information regarding the marker and commercially available antibodies to the marker are disclosed at genecards.org/cgi-bin/carddisp.pl?gene=CD34, last accessed on Aug. 13, 2014.

CD45 also is known as protein tyrosine phosphatase, receptor type C (PTPRC). A polynucleotide and amino acid sequence encoded by it is reported under GenBank number AY538691. Additional information regarding the marker and commercially available antibodies to the marker are disclosed at genecards.org/cgi-bin/carddisp.pl?gene=PTPRC, last accessed on Aug. 13, 2014.

The acronym “IL-8” intends “interleukin 8”. Information regarding IL-8 and antibodies to detect and quantify as well as commercially available assay kits are describe at genecards.org/cgi-bin/carddisp.pl?gene=IL8, last accessed on Aug. 13, 2014.

As used herein, the term “integrin receptor” or “integrin” intends the cell surface marker to which a ligand can bind.

As used herein, the term “differentiates or differentiated” defines a cell that takes on a more committed (“differentiated”) position within the lineage of a cell. “Dedifferentiated” defines a cell that reverts to a less committed position within the lineage of a cell.

As used herein, a “pluripotent cell” defines a less differentiated cell that can give rise to at least two distinct (genotypically and/or phenotypically) further differentiated progeny cells.

A “multi-lineage stem cell” or “multipotent stem cell” refers to a stem cell that reproduces itself and at least two further differentiated progeny cells from distinct developmental lineages. The lineages can be from the same germ layer (i.e. mesoderm, ectoderm or endoderm), or from different germ layers. An example of two progeny cells with distinct developmental lineages from differentiation of a multilineage stem cell is a myogenic cell and an adipogenic cell (both are of mesodermal origin, yet give rise to different tissues). Another example is a neurogenic cell (of ectodermal origin) and adipogenic cell (of mesodermal origin).

A “composition” is also intended to encompass a combination of active agent and another carrier, e.g., compound or composition, inert (for example, a detectable agent or label) or active, such as an adjuvant, diluent, binder, stabilizer, buffers, salts, lipophilic solvents, preservative, adjuvant or the like. Carriers also include biocompatible scaffolds, pharmaceutical excipients and additives proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. Carbohydrate excipients are also intended within the scope of this invention, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

“Substantially homogeneous” describes a population of cells in which more than about 50%, or alternatively more than about 60%, or alternatively more than 70%, or alternatively more than 75%, or alternatively more than 80%, or alternatively more than 85%, or alternatively more than 90%, or alternatively, more than 95%, of the cells are of the same or similar phenotype. Phenotype can be determined by a pre-selected cell surface marker or other marker.

A “biocompatible scaffold” refers to a scaffold or matrix for tissue-engineering purposes with the ability to perform as a substrate that will support the appropriate cellular activity to generate the desired tissue, including the facilitation of molecular and mechanical signaling systems, without eliciting any undesirable effect in those cells or inducing any undesirable local or systemic responses in the eventual host. In other embodiments, a biocompatible scaffold is a precursor to an implantable device which has the ability to perform its intended function, with the desired degree of incorporation in the host, without eliciting an undesirable local or systemic effects in the host. Biocompatible scaffolds are described in U.S. Pat. No. 6,638,369.

A neuron is an excitable cell in the nervous system that processes and transmits information by electrochemical signaling. Neurons are found in the brain, the vertebrate spinal cord, the invertebrate ventral nerve cord and the peripheral nerves. Neurons can be identified by a number of markers that are listed on-line through the National Institute of Health at the following website: “stemcells.nih.gov/info/scireport/appendixe.asp#eii,” and are commercially available through Chemicon (now a part of Millipore, Temecula, Calif.) or Invitrogen (Carlsbad, Calif.).

As used herein and known to the skilled artisan, a “marker” is a receptor or protein expressed by the cell or internal to the cell which can be used as an identifying and/or distinguishing factor. If the marker is noted as (“+”), the marker is positively expressed. If the marker is noted as (“−”), the marker is absent or not expressed. Variable expression of markers are also used, such as “high” and “low” and relative terms.

A neural stem cell is a cell that can be isolated from the adult central nervous systems of mammals, including humans. They have been shown to generate neurons, migrate and send out aconal and dendritic projections and integrate into pre-existing neuroal circuits and contribute to normal brain function. Reviews of research in this area are found in Miller (2006) Brain Res. 1091(1):258-264; Pluchino et al. (2005) Brain Res. Brain Res. Rev. 48(2):211-219; and Goh et al. (2003) Stem Cell Res. 12(6):671-679. Neural stem cells have previously been identified and isolated by neural stem cell specific markers including, but limited to, CD133, ICAM-1, MCAM, CXCR4 and Notch 1. Neural stem cells can be isolated from animal or human by neural stem cell specific markers with methods known in the art. See, e.g., Yoshida et al. (2006) Stem Cells 24(12):2714-22.

A “precursor” or “progenitor cell” intends to mean cells that have a capacity to differentiate into a specific type of cell. A progenitor cell may be a stem cell. A progenitor cell may also be more specific than a stem cell. A progenitor cell may be unipotent or multipotent. Compared to adult stem cells, a progenitor cell may be in a later stage of cell differentiation. An example of progenitor cell include, without limitation, a progenitor nerve cell.

A “neural precursor cell”, “neural progenitor cell” or “NP cell” refers to a cell that has a capacity to differentiate into a neural cell or neuron. A NP cell can be an isolated NP cell, or derived from a stem cell including but not limited to an iPS cell. Neural precursor cells can be identified and isolated by neural precursor cell specific markers including, but limited to, nestin and CD133. Neural precursor cells can be isolated from animal or human tissues such as adipose tissue (see, e.g., Vindigni et al. (2009) Neurol. Res. 2009 Aug 5. (Epub ahead of print)) and adult skin (see, e.g., Joannides (2004) Lancet. 364(9429):172-8). Neural precursor cells can also be derived from stem cells or cell lines or neural stem cells or cell lines. See generally, e.g., U.S. Patent Application Publications Nos. 2009/0263901, 2009/0263360 and 2009/0258421.

A nerve cell that is “terminally differentiated” refers to a nerve cell that does not undergo further differentiation in its native state without treatment or external manipulation. In one embodiment, a terminally differentiated cell is a cell that has lost the ability to further differentiate into a specialized cell type or phenotype.

A population of cells intends a collection of more than one cell that is identical (clonal) or non-identical in phenotype and/or genotype.

As used herein, the terms “disease” “disorder” and “condition” are used interchangeably, referring to an abnormal condition that negatively affects the structure or function of all or part of a subject. For example, a vascular disease, a neurological and/or neurodegenerative disease or a hyper-inflammation as disclosed herein.

The term neurodegenerative condition (or disorder) is an inclusive term encompassing acute and chronic conditions, disorders or diseases of the central or peripheral nervous system. A neurodegenerative condition may be age-related, or it may result from injury or trauma, or it may be related to a specific disease or disorder. Acute neurodegenerative conditions include, but are not limited to, conditions associated with neuronal cell death or compromise including cerebrovascular insufficiency, focal or diffuse brain trauma, diffuse brain damage, spinal cord injury or peripheral nerve trauma, e.g., resulting from physical or chemical burns, deep cuts or limb severance. Examples of acute neurodegenerative disorders are: cerebral ischemia or infarction including embolic occlusion and thrombotic occlusion, reperfusion following acute ischemia, perinatal hypoxic-ischemic injury, cardiac arrest, as well as intracranial hemorrhage of any type (such as epidural, subdural, subarachnoid and intracerebral), and intracranial and intravertebral lesions (such as contusion, penetration, shear, compression and laceration), as well as whiplash and shaken infant syndrome. Chronic neurodegenerative conditions include, but are not limited to, Alzheimer's disease, Pick's disease, diffuse Lewy body disease, progressive supranuclear palsy (Steel-Richardson syndrome), multisystem degeneration (Shy-Drager syndrome), chronic epileptic conditions associated with neurodegeneration, motor neuron diseases including amyotrophic lateral sclerosis, degenerative ataxias, cortical basal degeneration, ALS-Parkinson's-Dementia complex of Guam, subacute sclerosing panencephalitis, Huntington's disease, Parkinson's disease, synucleinopathies (including multiple system atrophy), primary progressive aphasia, striatonigral degeneration, Machado-Joseph disease/spinocerebellar ataxia type 3 and olivopontocerebellar degenerations, Gilles De La Tourette's disease, bulbar and pseudobulbar palsy, spinal and spinobulbar muscular atrophy (Kennedy's disease), primary lateral sclerosis, familial spastic paraplegia, Werdnig-Hoffmann disease, Kugelberg-Welander disease, Tay-Sach's disease, Sandhoff disease, familial spastic disease, Wohlfart-Kugelberg-Welander disease, spastic paraparesis, progressive multifocal leukoencephalopathy, familial dysautonomia (Riley-Day syndrome), and prion diseases (including, but not limited to Creutzfeldt-Jakob, Gerstmann-Straussler-Scheinker disease, Kuru and fatal familial insomnia), demyelination diseases and disorders including multiple sclerosis and hereditary diseases such as leukodystrophies.

Other neurodegenerative conditions include dementias, regardless of underlying etiology, including age-related dementia and other dementias and conditions with memory loss including dementia associated with Alzheimer's disease, vascular dementia, diffuse white matter disease (Binswanger's disease), dementia of endocrine or metabolic origin, dementia of head trauma and diffuse brain damage, dementia pugilistica and frontal lobe dementia. The term treating (or treatment of) a disorder/disease or condition refers to ameliorating the effects of, or delaying, halting or reversing the progress of, or delaying or preventing the onset of, a condition as defined herein. In one aspect, “treatment” is an improvement in locomotor function as compared to untreated controls, such as for example, the ability for self-care, to bear weight and/or become ambulatory (walk).

The term effective amount refers to a concentration or amount of a reagent or composition, such as a composition as described herein, cell population or other agent, that is effective for producing an intended result, including cell growth and/or differentiation in vitro or in vivo, or for the treatment of a disease/disorder/condition as described herein. It will be appreciated that the number of cells to be administered will vary depending on the specifics of the disorder to be treated, including but not limited to size or total volume/surface area to be treated, as well as proximity of the site of administration to the location of the region to be treated, among other factors familiar to the medicinal biologist.

The terms effective period (or time) and effective conditions refer to a period of time or other controllable conditions (e.g., temperature, humidity for in vitro methods), necessary or preferred for an agent or composition to achieve its intended result, e.g., the differentiation of cells to a pre-determined cell type.

The term patient or subject refers to animals, including mammals, such as bovines, canines, felines, ovines, equines, preferably humans, who are treated with the pharmaceutical compositions or in accordance with the methods described herein.

The term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present invention include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

A “control” is an alternative subject or sample used in an experiment for comparison purpose. A control can be “positive” or “negative”. For example, where the purpose of the experiment is to determine a correlation of an altered expression level of a gene with a particular phenotype, it is generally preferable to use a positive control (a sample from a subject, carrying such alteration and exhibiting the desired phenotype), and a negative control (a subject or a sample from a subject lacking the altered expression or phenotype). Additionally, when the purpose of the experiment is to determine if an agent effects the differentiation of a stem cell, it is preferable to use a positive control (a sample with an aspect that is known to affect differentiation) and a negative control (an agent known to not have an affect or a sample with no agent added).

The terms autologous transfer, autologous transplantation, autograft and the like refer to treatments wherein the cell donor is also the recipient of the cell replacement therapy. The terms allogeneic transfer, allogeneic transplantation, allograft and the like refer to treatments wherein the cell donor is of the same species as the recipient of the cell replacement therapy, but is not the same individual. A cell transfer in which the donor's cells and have been histocompatibly matched with a recipient is sometimes referred to as a syngeneic transfer. The terms xenogeneic transfer, xenogeneic transplantation, xenograft and the like refer to treatments wherein the cell donor is of a different species than the recipient of the cell replacement therapy.

As used herein, a “pluripotent cell” defines a less differentiated cell that can give rise to at least two distinct (genotypically and/or phenotypically) further differentiated progeny cells. In another aspect, a “pluripotent cell” includes an Induced Pluripotent Stem Cell (iPSC) which is an artificially derived stem cell from a non-pluripotent cell, typically an adult somatic cell, produced by inducing expression of one or more stem cell specific genes. Such stem cell specific genes include, but are not limited to, the family of octamer transcription factors, i.e., Oct-3/4; the family of Sox genes, i.e. Sox1, Sox2, Sox3, Sox 15 and Sox 18; the family of Klf genes, i.e. Klf1, Klf2, Klf4 and Klf5; the family of Myc genes, i.e. c-myc and L-myc; the family of Nanog genes, i.e. OCT4, NANOG and REX1; or LIN28. Examples of iPSCs are described in Takahashi et al. (2007) Cell advance online publication 20 Nov. 2007; Takahashi & Yamanaka (2006) Cell 126:663-76; Okita et al.(2007) Nature 448:260-262; Yu et al. (2007) Science advance online publication 20 Nov. 2007; and Nakagawa et al. (2007) Nat. Biotechnol. Advance online publication 30 Nov. 2007.

As used herein, the term “CRISPR” refers to a technique of sequence specific genetic manipulation relying on the clustered regularly interspaced short palindromic repeats pathway (CRISPR). CRISPR can be used to perform gene editing and/or gene regulation, as well as to simply target proteins to a specific genomic location. Gene editing refers to a type of genetic engineering in which the nucleotide sequence of a target polynucleotide is changed through introduction of deletions, insertions, or base substitutions to the polynucleotide sequence. In some aspects, CRISPR-mediated gene editing utilizes the pathways of nonhomologous end-joining (NHEJ) or homologous recombination to perform the edits. Gene regulation refers to increasing or decreasing the production of specific gene products such as protein or RNA.

The term “guide RNA” or “gRNA” as used herein refers to the guide RNA sequences used to target the CRISPR complex to a specific nucleotide sequence such as a specific region of a cell's genome. Techniques of designing gRNAs and donor therapeutic polynucleotides for target specificity are well known in the art. For example, Doench, J., et al. Nature biotechnology 2014; 32(12):1262-7, Mohr, S. et al. (2016) FEBS Journal 283: 3232-38, and Graham, D., et al. Genome Biol. 2015; 16: 260. gRNA comprises or alternatively consists essentially of, or yet further consists of a fusion polynucleotide comprising CRISPR RNA (crRNA) and trans-activating CRISPR RNA (tracrRNA); or a polynucleotide comprising CRISPR RNA (crRNA) and trans-activating CRISPR RNA (tracrRNA). In some aspects, a gRNA is synthetic (Kelley, M. et al. (2016) J of Biotechnology 233 (2016) 74-83).

The term “inhibitory RNA” refers to an RNA molecule capable of RNA interference, a mechanism whereby an inhibitory RNA molecule targets a messenger RNA (mRNA) molecule, resulting in inhibition gene expression and/or translation. RNA interference is also known as post-transcriptional gene silencing. Exemplary inhibitory RNAs include but are not limited to antisense RNAs, microRNAs (miRNA), small interfering RNAs (siRNA), short hairpin RNAs (shRNA), double stranded RNA (dsRNA) and intermediates thereof. Methods of designing, cloning, and expressing inhibitory RNAs are known in the art (e.g. McIntyre et al, BMC Biotechnol 2006; 6:1; Moore et al. Methods Mol Biol. 2010; 629: 141-158) and custom RNAi kits are commercially available (e.g. GeneAssist™ Custom siRNA Builder, ThermoFisher Scientific, Waltham, Mass.).

As used herein, the term “autologous,” in reference to cells refers to cells that are isolated and infused back into the same subject (recipient or host). “Allogeneic” refers to non-autologous cells.

As used herein, the term “extrusion” or any grammatical vacation thereof refers to a continuous process feeding materials through an extruder where the materials are pumped through a filter. In one embodiment, used herein are filters having pores of different size, such as a diameter of 400 nm to 50 nm. Such pore filters are then identified herein based on its diameters, for example, 200 nm filter refers to a filter having pores at a diameter of 200 nm. As used herein, suitable filters include but are not limited to 50 nm filter, 60 nm filter, 70 nm filter, 80 nm filter, 90 nm filter, 100 nm filter, 110 nm filter, 120 nm filter, 130 nm filter, 140 nm filter, 150 nm filter, 160 nm filter, 170 nm filter, 180 nm filter, 190 nm filter, 200 nm filter, 210 nm filter, 220 nm filter, 230 nm filter, 240 nm filter, 250 nm filter, 260 nm filter, 270 nm filter, 280 nm filter, 290 nm filter, 300 nm filter, 310 nm filter, 320 nm filter, 330 nm filter, 340 nm filter, 350 nm filter, 360 nm filter, 370 nm filter, 380 nm filter, 390 nm filter. In certain embodiments, extrusion may comprises extruding the materials through multiple filters. In a further embodiment, the multiple filters are applied to the materials based on their filter pore sizes, from the largest to the smallest.

### Modes for Carrying Out the Disclosure

Exosome-mimicking nanovesicles (EMNs) are produced by using isolated lipid-raft. The EMNs can be used to package biological materials such as stem cell secretome/cell-derived conditioned media and RNAs. Previous studies have used cell membrane vesicles to cloak liposomes or PLGA particles, however, this disclosure uses lipid rafts and/or plasma membrane to produce an EMN. These EMNs are completely cell derived and retain cell surface markers that likely help in the targeted delivery of these vesicles to specific cells. Also, the EMNs here can be further personalized (autologous therapy) and possess the surface receptors for targeted delivery. The same kind of vesicles can be produced from artificial lipids, however, their surfaces often require modification targeted delivery and use in the body.

Several non-limiting examples are provided in this application for illustration purposes. One example shows that the exosome-mimicking nanovesicles containing the concentrated conditioned media is able to improve the recovery of apoptotic neurons in culture. Briefly, Applicant cultured PMSCs, isolated lipid rafts and/or plasma membrane (such as via density gradient centrifugation), characterized lipid rafts and/or plasma membrane (such as via Western blotting screening raft/exosome specific makers), obtained and analyzed and concentrated conditioned media, synthesized EMNs (for example, containing conditioned media using Mini Extruder), determined loading efficiency and evaluated the synthesized EMNs (e.g., via Transmission electron microscopy (TEM) showing morphology, via nanoparticle tracking analysis (NTA) providing size distribution and concentration, and via plate reader quantify uptake of a detectable cargo (such as FITC-BSA) using fluorescent plate reader assay; and performed functional assay (e.g., neuroprotection assay such as WimNeuron Analysis analyzing neurite outgrowth, branching and circuitry length) exhibiting EMNs' neuroprotective functions on apoptotic neurons.

Another example focuses on vascular related condition. Extracellular vesicles (EVs) are small nanovesicles derived from the invagination of the cell plasma membranes that function as primary messengers of intercellular communication (Thery et al., Nat. Rev. Immunol, (2002); Chang et al., Cell Biosci, (2019); Colombo et al., (2014)). EVs can be secreted by all types of cells that have shown great promise as noninvasive nanotherapeutics for regenerative medicine (Thery et al., Nat. Rev. Immunol, (2002); Chang et al. Cell Biosci, (2019); Colombo et al., (2014)). Applicant showed that EVs derived from placental mesenchymal stromal cells have significant neuroprotective and immunomodulatory properties that make them a viable treatment option for neurodegenerative disorders (Kumar et al., (2019); Clark et al., (2019). Particularly, placental MSCs secretions include free proteins, such as (brain-derived neurotrophic factor (BDNF), hepatocyte growth factor (HGF), and vascular endothelial growth factors (VEGF)) as well as exosomes and have neuroprotective functions. See, Kumar et al. (2019), Clark et al. (2019), and Chen et al. (2017). Similarly, many other studies have shown that exosomes, a subclass of EVs, derived from endothelial progenitor cells (EPCs) exhibit significant angiogenic potential. Multiple studies have characterized their ability to promote endothelial cell proliferation, migration, and tube formation (Li et al., Cytotherapy, (2016); Li et al., Journal of Diabetes and its Complications (2016); Wu et al., Experimental Cell Research (2018); Mathiyalagan et al., Circ Res, (2017)). Without wishing to be bound by the theory, this is mainly due to their internal cargo, which contains abundant levels of miR126, a highly proangiogenic miRNA that is known to promote vascularization and attenuates levels of inflammatory cytokines and chemokines (Wu et al., Experimental Cell Research, (2018); Zhou et al., Molecular Therapy, (2018)). miR126 has also been seen to facilitate the recruitment of endogenous circulating EPCs and stimulate maturation into functional endothelial phenotypes (Fish et al., Developmental Cell, (2008); Sessa et al., Biochim. Biophy. Acta, (2012)) all the while preventing vascular smooth muscle cell proliferation and migration to limit neointimal hyperplasia (Jansen et al., Journal of Molecular and Cellular Cardiology, (2017); Izuhara et al., PLoS ONE, (2017)). Furthermore, emerging data on EV membrane properties also reveal the presence of unique proteins that impart highly specific targeting properties to the EVs following cell secretion (Deng et al., Cellular Reprogramming, (2018); Murphy et al., Exp Mol Med, (2019)). Together, EV membrane composition and proteins are key in protecting internal cargo from degradation by enzymes and in facilitating proper EV uptake by target cell. All in all, EVs present as a biological and multifunctional therapeutic and treatment for a variety of diseases and defects. However, EV application for clinical translation has been greatly limited due to difficulties in EV isolation and purification. Obtaining EVs, especially from cell cultures, is an extremely time-consuming, laborious, and costly process [Zhang et al., Cell Biosci, (2019); Li et al., APL Bioeng, (2019)). Preferentially sorting functional subpopulations of therapeutic EVs from other vesicle types is also difficult, and thus the isolated EV fractions contain unwanted populations of vesicles (Li et al., APL Bioeng, (2019)). Additionally, there is an inherent heterogeneity of functional properties between EVs of similar cell origin due to the sensitivity of EV secretion and properties in response to cellular environment (Thery et al., Nat. Rev. Immunl, (2002); Colombo et al., (2014)). With the difficulties in obtaining and standardizing EVs, the therapeutic application of these nanovesicles has become a major challenge. Therefore, rather than using native EVs as vascular therapeutics, Applicant instead sought to develop an EV-inspired nanotherapeutic that can mimic the functional and physical properties of native EVs. As a proof-of-concept, Applicant sought to mimic EPC EVs in order to promote vascularization and reendothelization, both of which are common regenerative processes involved in the treatment of many diseases and disorders. This EPC EV-mimic (EPC-EM) consists of a miR126-loaded poly (lactic-co-glycolic acid) (PLGA) nanoparticle that will be coated with EPC-derived plasma membrane (PM) and functionalized with a collagen-binding peptide SILY. Applicant designed this EPC-EM for multiple functions: (1) target exposed collagen and prevent platelet adhesion and activation to decrease early and late-stage thrombosis, (2) promote reendothelialization and vascularization by upregulating angiogenic genes in endothelial cells, and (3) limit neointimal hyperplasia by suppressing overactive vascular smooth muscle cell proliferation and migration. Without wishing to be bound by the theory, these functions are directed by the main components of the EPC-EM design.

miRNAs (e.g. miR126), which is be used to stimulate EPC and EC migration and proliferation for reendothelization and modulate smooth muscle cell function to prevent neointimal hyperplasia. A potent proangiogenic microRNA, miR126, has been well characterized for its proangiogenic properties as well its ability to modulate smooth muscle cell function (Fish et al., Developmental Cell, (2008); Jansen et al., Journal of Molecular and Cellular Cardiology, (2017); Izuhara et al. PLoS ONE, (2017)). Additionally, miRNA-loaded PLGA nanoparticles have been previously established in the field of nanomedicine (Anata et al. Cells. Mol. Pharmaceutics, (2015); Devulapally et al., ACS Nano, (2015); Devalliere et al., The FASEB Journal, (2014); Tsumaru et al., Journal of Vascular Surgery, (2018). It is necessary to encapsulate miRNA within delivery systems as synthetic naked miRNAs are quickly degraded by circulating nucleases in plasma. Unlike cationic lipid polymers that have been traditionally used to encapsulate and deliver nucleic acids, PLGA is noncytotoxic, provides greater stability, and has tailorable release kinetics (Sharma et al. Biomaterials, (2011)). Therefore, PLGA is an ideal polymer biomaterial that can retain miRNA and provide stability for the EPC-EM design.

EPC PM, which has shown potential to mediate many biological processes, including angiogenesis and platelet adhesion. Use of EPC PM can mimic the physical membranous structure of EVs. EVs, including exosomes, are composed of membrane lipids and proteins due their eventual secretion through the plasma membrane of cells. This membranous structure can potentially be mimicked by coating isolated plasma membrane onto the PLGA core. EPC plasma membrane additionally has important transmembrane proteins that can help mediate platelet adhesion and angiogenic processes.

Ligands (e.g. SILY), which will be used for EPC-EM targeting and localization to the exposed collagen at injured vascular sites. SILY is an ideal peptide due to its strong binding affinity to collagen (Paderi et al., Biomaterials, (2011); McMasters et al., Acta Biomaterialia, (2017)). Derived from a platelet-receptor for collagen, SILY also directly competes with platelets for preferential binding to exposed collagen at damaged sites. By preventing platelet binding, SILY can inhibit the initiation of the platelet cascade and limit adverse immune responses.

By combining the described components into a single nanotherapeutic system, Applicant sought to engineer a synthetic EV that can inhibit platelet binding and promote vascularization. Without wishing to be bound by the theory, the EPC-EM mechanism of action in vivo occurs in two ways. First, EPC-EM particles localize and bind to the exposed collagen at injured sites, where it prevents platelet adhesion and releases encapsulated miR126 to modulate vascular smooth muscle cell and endothelial cell (EC) behavior. Second, any unbound or excess EPC-EM particles also follow more conventional EV mechanism of action and are uptaken by adjacent ECs and circulating EPCs to promote vascularization at the injured sites.

Provided herein is an exosome mimicking nanovesicle (EMN) comprising cell-derived lipid rafts and/or plasma membrane and substantially devoid of native exosomes. The EMN can be derived from a differentiated cell, a partially differentiated cell, or a stem cell. They can be derived from any specifies of cell having a cellular membrane such as animal cell, mammalian cells, e.g., canine, equine, feline, ovine, and human. In one aspect the EMN is derived from an adult stem cell, non-limiting examples of such include a neuronal stem cell, a mesenchymal stem cell, an adipose derived stem cell and an induced pluripotent stem cell (iPSC), and optionally wherein the mesenchymal stem cell expresses CD105⁺, CD90⁺, CD73⁺, CD44⁺ and CD29⁺ and optionally CD184+. In a further aspect, the stem cell is a mesenchymal stem cell expresses exosome specific markers CD9, CD63, ALIZ, TSG101 and the alpha 4 and beta 1 integrin. In a further aspect, the stem cell is a human stem cell that expresses CD105⁺, CD90⁺, CD73⁺, CD44⁺ and CD29⁺ and optionally CD184+. In a further aspect, the stem cell is a human mesenchymal stem cell expresses exosome specific markers CD9, CD63, ALIZ, TSG101 and the alpha 4 and beta 1 integrin. The cells that are used to derive the EMNs can be isolated or of the type from a pediatric, fetal or pre-term placenta-derived stem cell. Alternatively, they can be derived from appropriate cell lines or they can be from recently isolated tissue subject to minimal passages (P0 to P4, for example), prior to manipulation.

The EMNs of this disclosure can further comprise a stem cell derived secretome. In addition, or alternatively, the EMNs of this disclosure can further comprise an exogenous agent selected from a polynucleotide, a peptide, a protein, an antibody fragment, or a therapeutic agent (e.g., a small molecule or biologic). Non-limiting examples include a polynucleotide selected from an inhibitory RNA, a therapeutic gene or a CRISPR system. In addition, or alternatively, the EMN comprises serum albumin and biotin.

The EMNs can be combined into populations, wherein the EMNs can be the same or different from each other in terms of cell derivation, cell type, concentration or identity of contents, or size of the plurality of EMNs. Alternatively, the plurality of EMNs can be substantially identical or identical to each other in terms of cell derivation, cell type, concentration or identity of contents, or size of the plurality of EMNs. The individual EMN and populations can be combined with a carrier, such as a pharmaceutically acceptable carrier. The carrier can be a modified for the intended use, e.g., a biocompatible matrix or scaffold or a liquid carrier. Alternatively, the carrier is selected from a hydrogel, a thixotropic agent, a phase changing agent, a collagen gel, a collagen gel, an extracellular matrix (ECM), an amnion patch, a nanofiber scaffold (aligned and nonaligned) and fibrin glue.

The EMNs and compositions can be used to rescue a neuron by a method comprising, or alternatively consisting essentially of, or yet further consisting of, administering an effective amount of the EMN or plurality to the neuron. In one aspect the neuron is an apoptotic neuron and the method is used to rescue an apoptotic neuron. The administration can be in vitro to a neuron in an ex vivo environment or in vivo by administration to a cell or tissue in a subject. The subject and neuron can be from any animal species, e.g., a canine, an equine, a feline, an ovine, a simian or a human patient. The subject can be a fetus, an infant, a child or an adult. The EMN can be autologous or allogeneic to the subject or cell being treated. Administration can be local or systemic, as the need may be. They can be administered in a pharmaceutically acceptable carrier or a biocompatible matrix. In one aspect, the subject is a fetus and the EMN, or EMN composition is administered to the fetus in utero. In one embodiment, the administration is an inhaled/intranasal administration. In a further embodiment, the administration is via aerosol inhalation.

In one aspect, provided is an exosome mimicking nanovesicle (EMN) comprising a shell encapsulating a cargo. In one embodiment, the shell comprises a plasma membrane. Additionally or alternatively, the shell and/or the EMN comprises a lipid raft. In a further embodiment, the EMN is substantially devoid of (or substantially free of) native exosomes. In yet a further embodiment, the shell and/or the EMN comprises an artificial lipid. As used herein, the term “artificial lipid” intends a lipid composition whose source is not directly from a natural source, e.g., a cell or tissue.

In one embodiment, the shell is derived from or isolated from a cell capable of secreting an exosome. In yet a further embodiment, the EMN comprises a core encapsulated in the shell with the cargo. Additionally or alternatively, the shell further comprises a peptide or a protein, which is referred to herein as a shell peptide or a shell protein. In one embodiment, the EMN further comprises a scaffold.

In certain embodiments, a cargo of the EMN as disclosed herein comprises an exogenous agent. In a further embodiment, the exogenous agent is selected from a polynucleotide, a peptide, a protein, an antibody fragment, a small molecule or a therapeutic agent.

In one embodiment, the polynucleotide is selected from a RNA, a DNA, an inhibitory RNA, an miRNA, an siRNA, a therapeutic gene or a CRISPR system. In a further embodiment, the miRNA is one or more of the following: hsa-miR-138-5p, hsa-miR-22-5p, miR-218-5p, hsa-let-7b-5p, hsa-let-7f-5p, hsa-miR-122-5p, hsa-let-7g-5p, hsa-let-7i-5p, hsa-miR-22-5p, hsa-miR-186-5p, hsa-let-7d-5p, hsa-miR-19a-3p, hsa-mir-98, hsa-let-7c, or hsa-miR-29a-3p. In yet a further embodiment, the cargo comprises a miRNA and a cationic counterion (such as spermidine). In one embodiment, the cargo comprises a complex comprising an hsa-miR126-3p and a cationic counterion (such as spermidine). In another embodiment, the cargo comprises a complex of an hsa-miR126-3p and a cationic counterion (such as spermidine). In a further embodiment, the polynucleotide further comprises a regulatory sequence which directs the expression of the RNA or DNA. In one embodiment, the polynucleotide (such as a therapeutic gene) is about 3 nucleotides (nt) to one of the following: less than about 500 nt, or less than about 1000 nt, or less than about 2000 nt, or less than about 3000 nt, or less than about 4000 nt, or less than about 5000 nt, or less than about 6000 nt, or less than about 7000 nt, or less than about 8000 nt, or less than about 9000 nt, or less than about 10000 nt, or less than about 15000 nt, or less than about 20000 nt, or less than about 30000 nt, or less than about 40000 nt, or less than about 50000 nt, or less than about 60000 nt, or less than about 70000 nt, or less than about 80000 nt, or less than about 90000 nt. In a further embodiment, the therapeutic agent is a gene encoding a polynucleotide encoding a B-cell lymphoma/leukemia 11A.

In one embodiment, the cargo comprises a peptide or a protein, that is optionally selected from one or more of a growth factor, a chemokine, or a cytokine. In a further embodiment, the growth factor is selected from the group of: a platelet-derived growth factor, a hepatocyte growth factor (HGF), a brain-derived neurotropic factor (BDNF), or a vascular endothelial growth factors (VEGF) or a combination thereof. In yet a further embodiment, the chemokine or cytokine is selected from the group of: a monocyte chemoattractant protein-1 (MCP-1), IL-8, or IL-6 or a combination thereof. Additionally or alternatively, the cargo comprises a peptide or a protein that optionally selected from the group of: HGF, BDNF, VEGF, galectin 1, MCP-1, IL-8, IL-6, a-catenin, b-catenin, platelet-derived growth factor, TGF-β, Wnt5a, tissue factor, integrin a4b1, MMP1, MMP2, MMP14, ADAM9, ADAM10, ADAM17, a disintegrin and metalloprotease (for example, ADAM), matrix metalloproteinase (MMP), or TIMP (optionally a tissue inhibitor of metalloproteinase, for example TIMP 1, TIMP-2, or TIMP-3) BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TNF-α, or a combination thereof.

In certain embodiments, the cargo comprises a cell derived conditioned medium. In one embodiment, the cargo comprises one or more of the following: platelet-derived growth factor, hepatocyte growth factor (HGF), brain-derived neurotropic factor (BDNF), vascular endothelial growth factors (VEGF), Bone morphogenetic proteins (BMPs), Ciliary neurotrophic factor (CNTF), Epidermal growth factor (EGF), Macrophage colony-stimulating factor (M-CSF), Granulocyte colony-stimulating factor (G-CSF), Granulocyte macrophage colony-stimulating factor (GM-CSF), Ephrin A1, Ephrin A2, Ephrin A3, Ephrin A4, Ephrin A5, Ephrin B1, Ephrin B2, Ephrin B3, Erythropoietin (EPO), Fibroblast growth factor (FGF), Growth differentiation factor-9 (GDF9), Hepatoma-derived growth factor (HDGF), Insulin-like growth factors, Interleukin, Keratinocyte growth factor (KGF), Migration-stimulating factor (MSF), Macrophage-stimulating protein (MSP), Neuregulin, Nerve growth factor (NGF), Neurotrophin-3 (NT-3), Neurotrophin-4 (NT-4), Placental growth factor (PGF), Platelet-derived growth factor (PDGF), T-cell growth factor (TCGF), Thrombopoietin (TPO), Transforming growth factor alpha (TGF-α), Transforming growth factor beta (TGF-β), or Tumor necrosis factor-alpha (TNF-α). In a further embodiment, the cargo comprises a cell derived conditioned medium in addition to one or more of the following: HGF, BDNF, VEGF, BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TGF-β, or TNF-α. In another embodiment, the conditioned medium comprises one or more of the following: HGF, BDNF, VEGF, BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TGF-β, or TNF-α.

In certain embodiments, the cargo comprises one or more of proteins or polypeptides having a molecular weight from about 1 Da to about 1000 kDa. In one embodiment, the cargo comprises one or more of proteins or polypeptides having a molecular weight less than about 1000 kDa, or about 900 kDa, or about 800 kDa, or about 700 kDa, or about 600 kDa, or about 500 kDa, or about 400 kDa, or about 300 kDa, or about 200 kDa, or about 100 kDa. Additionally or alternatively, the cargo comprises one or more of proteins or polypeptides having a molecular weight more than about 1 Da, or about 2 Da, or about 10 Da, or about 50 Da, or about 100 Da, or about 200 Da, or about 300 Da, or about 400 Da, or about 500 Da, or about 600 Da, or about 700 Da, or about 800 Da, or about 900 Da, or about 1 kDa, or about 2 kDa, or about 3 kDa, or about 4 kDa, or about 5 kDa, or about 6 kDa, or about 7 kDa, or about 8 kDa, or about 9 kDa, or about 10 kDa, or about 10 kDa, or about 20 kDa, or about 30 kDa, or about 40 kDa, or about 50 kDa, or about 60 kDa, or about 70 kDa, or about 80 kDa, or about 90 kDa, or about 100 kDa. In one embodiment, the cargo comprises one or more of proteins or polypeptides having a molecular weight is selected from the following: from about 1 Da to about 1000 kDa, from about 10 Da to about 1000 kDa, from about 100 Da to about 1000 kDa, from about 1 kDa to about 1000 kDa, from about 1 Da to about 500 kDa, from about 10 Da to about 500 kDa, from about 100 Da to about 500 kDa, from about 1 kDa to about 500 kDa, from about 1 Da to about 400 kDa, from about 10 Da to about 400 kDa, from about 100 Da to about 400 kDa, from about 1 kDa to about 400 kDa, from about 1 Da to about 300 kDa, from about 10 Da to about 300 kDa, from about 100 Da to about 300 kDa, from about 1 kDa to about 300 kDa, from about 1 Da to about 200 kDa, from about 10 Da to about 200 kDa, from about 100 Da to about 200 kDa, from about 1 kDa to about 200 kDa, from about 1 Da to about 100 kDa, from about 10 Da to about 100 kDa, from about 100 Da to about 100 kDa, from about 1 kDa to about 100 kDa.

In certain embodiments, the core is selected from the group of a polymer core, optionally wherein the core is selected from the group of poly(l-lysine) (PLL), polyethylenimine (PEI), polyamidoamines, polyimidazoles, poly(ethylene oxide), polyalkylcyanoacrylates, polylactide, polylactic acid (PLA), poly-ε-caprolactone (PCL), poly (lactic-co-glycolic acid) (PLGA), silica, alginate, cellulose, pullulan, gelatin, or chitosan. In one embodiment, the core comprises a PLGA core and the plasma membrane to PLGA weight ratio is from about 1:10 to about 10:1, optionally about 1:20, or about 1:8, or about 1:5, or about 1:4, or about 1:3, or about 1:2, or about 1:1, or about 2:1, or about 3:1, or about 4:1, or about 5:1, or about 6:1, about 8:1, or about 10:1. Additionally or alternatively, N/P ratio of a complex comprising a cargo loaded on PLGA and/or an EMN comprising a cargo and a PLGA core is from 100:1 to 1:1, or from 50:1 to 1:1 , from 20: 1 to 1:1, about 15:1, about 10:1, about 11:1, about 12:1, about 13:1, about 14:1, about 20:1, about 19:1, about 18:1, about 17:1, or about 16:1.

In certain embodiments, the cargo and/or shell comprises a peptide or a protein that optionally selected from the group of: HGF, BDNF, VEGF, galectin 1, MCP-1, IL-8, IL-6, a-catenin, b-catenin, platelet-derived growth factor, TGF-β, Wnt5a, tissue factor, integrin a4b1, MMP1, MMP2, MMP14, ADAM9, ADAM10, ADAM17, a disintegrin and metalloprotease (for example, ADAM), matrix metalloproteinase (MMP), or TIMP (optionally a tissue inhibitor of metalloproteinase, for example TIMP 1, TIMP-2, or TIMP-3) BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TNF-α, or a combination thereof.

In certain embodiments, the shell peptide or protein facilitates one or more of the following: targeting the EMN to a cell and/or tissue, penetrating a cell, modulating immunoregulatory activity, or protecting a cell. In a further embodiment, the shell peptide or protein is selected from the following: a collagen-binding ligand, a platelet-receptor for collagen, an inhibitor of platelet reactivity, SILY (RRANAALKAGELYKSILYGC, SEQ ID NO: 1), CD39; a cell-penetrating peptide; a cell-targeting peptide; a human leukocyte antigen-G (HLA-G); Galectin1 or a combination thereof. In yet a further embodiment, the peptide or protein is conjugated to the shell covalently or non-covalently, directly or indirectly via a linker. In one embodiment, the peptide or protein is conjugated to the shell via one or more of the following: Click chemistry, DOPE-PEG-peptide, DOPE-NHS-peptide chemistry, biotin-streptavidin linkage, or peptide-peptide linkage. In another embodiment, the peptide or protein is conjugated via using hosphatidylethanolamines, such as DSPE, DMPE, DPPE, or DOPE. In yet another embodiment, the peptide or protein is conjugated to the shell via biotin-streptavidin linkage or peptide-peptide linkage. In one embodiment, the peptide or protein covalently binds an azide group to an alkyne moiety using a triazole linkage. In another embodiment, DBCO-sulfo-NHS comprises a biochemical linker to conjugate a modified azide-SILY to the shell via sulfo-NHS ester and Click chemistry.

In certain embodiments, the scaffold is selected from the group of: a graft, a stent, a medical material, an implant, a transplant, or a medical device.

In certain embodiments, the shells or cargos are the same or different from each other. In another embodiment, the shells and cargos are the same or different from each other.

In another aspect, provided is a composition comprising a carrier and an EMN as disclosed herein and/or a plurality of EMNs. In certain embodiments, the shells or cargos are the same or different from each other. In another embodiment, the shells and cargos are the same or different from each other. In one embodiment, the plurality further comprises EMNs comprising serum albumin and/or biotin as the cargo.

In one embodiment, provided is an EMN comprising lipid rafts derived from human placenta MSCs (hPMSCs) in/as the shell and hPMSCs-derived condition medium as cargos. In another embodiment, provided is an EMN comprising endothelial progenitor cell (EPC) derived plasma membrane in/as the shell and miR126 as a cargo, and optionally the cargo is loaded to a PLGA core before encapsulated by the shell.

Also provided is a method for treating a damaged neuron, comprising contacting the neuron with an effective amount of an EMN or plurality of EMN as described herein to the damaged neuron. In one aspect, the neuron is an isolated cortical neuron or a spinal cord neuron. The contacting can be in vitro or in vivo. Alternatively, the administration can be in vitro to a neuron in an ex vivo environment or in vivo by administration to a cell or tissue in a subject. The subject and neuron can be from any animal species, e.g., a canine, an equine, a feline, an ovine, a simian or a human patient. The subject can be a fetus, an infant, a child or an adult. The EMN can be autologous or allogeneic to the subject or cell being treated. Administration can be local or systemic, as the need may be. They can be administered in a pharmaceutically acceptable carrier or a biocompatible matrix. In one aspect, the subject is a fetus and the EMN, or EMN composition is administered to the fetus in utero. The method of is useful to treat a neuron by a neurodegenerative disease or disorder, an ischemic brain injury, a moderate or a catastrophic brain injury, a chemical neurotoxin exposure, a spinal cord injury, a traumatic brain injury, Parkinson's disease or a spinal cord contusion.

Also provided by this disclosure is a method for treating one or more of: Myelomeningocele (MCC), spina bifida, spinal cord injury or paralysis, in a subject in need thereof comprising administering to a subject in need thereof an effective amount of an EMN or a plurality of EMN, as described herein. The subject and neuron can be from any animal species, e.g., a canine, an equine, a feline, an ovine, a simian or a human patient. The subject can be a fetus, an infant, a child or an adult. The EMN can be autologous or allogeneic to the subject or cell being treated. Administration can be local or systemic, as the need may be. They can be administered in a pharmaceutically acceptable carrier or a biocompatible matrix. In one aspect, the subject is a fetus and the EMN, or EMN composition is administered to the fetus in utero.

In yet another aspect, provided is a method for rescuing a cell comprising administering an effective amount of an EMN as disclosed herein, and/or a plurality of to the cell as disclosed herein. In one embodiment, the cell is selected from the group of: a neuron, an endothelial cell, or a lung cell. Additionally or alternatively, the administration is in vitro or in vivo. In another embodiment, the administration is in vivo and the cell is a mammalian cell.

In a further aspect, provided is a method for preventing or treating one or more of: vascular diseases, neuronal diseases, or a hyper-inflammation in a subject in need thereof comprising administering to a subject in need thereof an effective amount of an EMN of as disclosed herein, and/or a plurality of EMNs as disclosed herein. In one embodiment, the vascular diseases are selected from the group of hind limb ischemia or cardiac ischemia. In another embodiment, the neuronal diseases are selected from the group of a neurodegenerative disease or disorder, an ischemic brain injury, stroke, a moderate or a catastrophic brain injury, a chemical neurotoxin exposure, a spinal cord injury, a traumatic brain injury, Alzheimer's disease, Parkinson's disease or a spinal cord contusion, spina bifida, myelomeningocele (MCC), multiple sclerosis, demyelination, oligodendroglia degeneration, lack of oligodendrocyte precursor cell (OPC) differentiation, or paralysis. In yet another embodiment, the hyper-inflammation is caused by a viral, bacterial, fungal or parasitic infection. In a further embodiment, the infection is a coronavirus infection, such as severe acute respiratory syndrome (SARS) coronavirus (SARS-CoV), SARS-CoV-2 causing the novel coronavirus disease-2019 (COVID-19), or Middle East respiratory syndrome (MERS) coronavirus (MERS-CoV). In yet another embodiment, the hyper-inflammation is caused by an acute respiratory distress syndrome (ARDS), a virus induced ARDS, a pneumonia, or a drug treatment, further optionally wherein the drug treatment is selected from administering an antibody or a fragment thereof, a gene therapy (such as administering an AAV viral vector or an HSV), or a cell therapy (such as an adoptive T-cell therapy, an adoptive NK-cell therapy, or an adoptive macrophage therapy, administering CAR-T cells, CAR-NK cells and/or CAR-macrophages).

In yet a further aspect, provided is a method for treating a damaged cell or preventing the cells from being damaged comprising contacting the cell with an effective amount of an EMN as disclosed herein, and/or a plurality of EMNs as disclosed herein to the damaged cell. In one embodiment, the cell is selected from neurons, endothelial cells, or lung cells. In another embodiment, the contacting is in vitro or in vivo. In one embodiment, the neuron to be treated is damaged by a neurodegenerative disease or disorder, such as an ischemic brain injury, stroke, a moderate or a catastrophic brain injury, a chemical neurotoxin exposure, a spinal cord injury, a traumatic brain injury, Alzheimer's disease, Parkinson's disease or a spinal cord contusion, spina bifida, myelomeningocele (MCC), multiple sclerosis, demyelination, oligodendroglia degeneration, lack of oligodendrocyte precursor cell (OPC) differentiation, paralysis, or a hyper-inflammation. In another embodiment, the endothelial cell is damaged in a vascular disease, an ischemia, a cardiovascular disease, hind limb ischemia, cardiac ischemia, or a hyper-inflammation. In yet another embodiment, the lung cell is damaged by a hyper-inflammation, optionally caused by an acute respiratory distress syndrome (ARDS), a virus induced ARDS, or a pneumonia. In one embodiment, the hyper-inflammation is caused by a viral, bacterial, fungal or parasitic infection, optionally a coronavirus infection. In a further embodiment, the coronavirus is selected from severe acute respiratory syndrome (SARS) coronavirus (SARS-CoV), SARS-CoV-2 causing the novel coronavirus disease-2019 (COVID-19), or Middle East respiratory syndrome (MERS) coronavirus (MERS-CoV). In one embodiment, the hyper-inflammation is optionally due to a drug treatment. In a further embodiment, the drug treatment is selected from administering an antibody or a fragment thereof, a gene therapy, or a cell therapy. In one embodiment, the gene therapy is an adeno-associated virus therapy, and the cell therapy is selected from the group of an adoptive T-cell therapy, an adoptive NK-cell therapy, or an adoptive macrophage therapy.

In certain embodiments, particularly those relating to administration, the EMN and/or plurality of EMNs is administered in a pharmaceutically acceptable carrier or biocompatible matrix.

In certain embodiments, particularly those relating to administration, Administration can be local or systemic, as the need may be. In one embodiment, the administration is inhalation, intravenous, intrathecal, intraspinal, intrapulmonary, intranasal, epidural, oral, or intraamniotic fluid. In another embodiment, the subject is a fetus and the composition is administered to the fetus in utero. In yet another embodiment, the administration is via aerosol inhalation.

In certain embodiments, particularly those relating to administration, the EMNs are administered with a pharmaceutically acceptable carrier or biocompatible matrix, that is optionally selected from a hydrogel, a thixotropic agent, a phase changing agent, a collagen gel, a collagen gel, an extracellular matrix (ECM), an amnion patch, a nanofiber scaffold (aligned and nonaligned) and fibrin glue.

Also provided is a method of producing of an EMN as disclosed herein and/or a plurality as disclosed herein. This production method allow obtaining a therapeutically significant and clinically relevant number of exosomes, i.e., the EMN yield of this method is large enough for producing a therapeutic composition for treating a cell, tissue, and/or a subject. As shown in the Examples, the production method disclosed herein generates exosomes at a much higher level compared to the method of producing an exosome currently available in the field (for example, culturing a cell followed/accompanied by collecting and purifying native exosomes generated).

The method comprises the following: (i) optionally hypotonically lyse cells; (ii) an optional mechanical homogenization; (iii) isolate or purify the lipid rafts and/or plasma membrane from the cell, optionally via one or more of centrifugation, optionally at the same or different relative centrifugal forces, optionally using serial ultracentrifugation and collecting materials at the density of lipid rafts and/or plasma membrane; and (iv) extrude the lipid rafts and/or plasma membrane with a solution comprising cargos using an extruder, optionally the extruder comprises a filter selected from an about 50 nm to 300 nm filter, optionally an about 200 nm filter, an about 150 nm filter, an about 100 nm filter, whereby generating EMNs comprising a cargo and lipid rafts and/or plasma membrane; or (v) extrude the lipid rafts and/or plasma membrane using an extruder, optionally the extruder comprises a filter selected from an about 50 nm to 300 nm filter, optionally an about 200 nm filter, an about 150 nm filter, an about 100 nm filter, centrifuge the extruded materials, remove supernatant and resuspend the rest martials comprising lipid rafts and/or plasma membrane using a solution comprising cargos, whereby EMNs were self-assembled from the extruded lipid rafts and/or plasma membrane encapsulating a cargo.

In one embodiment, the cargo is selected from cell-derived medium, BSA, biotin or any other protein(s) having a molecular size similar to a BSA and/or a biotin (for example, from 1 kDa to about 500 kDa, from 1 kDa to about 250 kDa, from 1 kDa to about 200 kDa). In one embodiment, the cargo is an miRNA. In a further embodiment, the cargo is loaded on a core, such as PLGA. In one embodiment, the EMN comprises cell-derived lipid rafts and/or cell-derived plasma membrane.

In one embodiment, the method further comprises one or more steps of producing, washing, isolating and/or purifying the cargo. Additionally or alternatively, the method further comprises one or more steps of washing, isolating and/or purifying the lipid rafts and/or plasma membrane. In another embodiment, the method further comprises one or more steps of washing, isolating and/or purifying the produced EMNs comprising the cargo. In one embodiment, the cargo is loaded to a core before or during its encapsulation into an EMN shell. In a further embodiment, the method further comprises one or more steps of producing, washing, isolating and/or purifying the core. Additionally or alternatively, the method further comprises loading the cargo to a core. In a further embodiment, the method further comprises one or more of washing, isolating and/or purifying the core loaded with the cargo. Techniques and methods for such washing, isolating and/or purifying steps, as well as the step of producing cargo, core and/or cargo-loaded core, are disclosed herein. See, Examples 1 and 2. Other techniques and methods are known in the art, see for example, Ramasubramanian et al. (2019).

In one embodiment, the cells are selected from the group of: a differentiated cell; a stem cell; a cancer cell; or an immune cell: neutrophils, eosinophils, basophils, mast cells, monocytes, macrophages, dendritic cells, natural killer cells, and lymphocytes (B cells and T cells). In another embodiment, the cell can be any cell as disclosed herein or any combination thereof.

In a further embodiment, if the cargo is a cell derived condition medium, the method further comprises culturing the cell, collect culture medium used for culturing the cell, and optionally isolating, purifying and/or condensing the collected medium. In one embodiment, the cell from which the conditioned medium is derived from may be the same cell providing the lipid rafts/plasma membrane. In another embodiment, the cell from which the conditioned medium is derived from is different from the cell providing the lipid rafts/plasma membrane. In either embodiment, the cells can be any type as disclosed herein or any combination thereof.

This method is fully scalable, for example via performing each step in large scale, and/or in a continuous manner without interruption. One non-limiting example is, without disrupting the overall cell culture, culturing cells in a bioreactor, replenishing culture medium continuously, and at the same time, collecting cells for isolating lipid rafts/plasma membrane as needed. In one embodiment, lipid raft is collected based on its density, for example having a density falls within the range from the density of solution Gradient 4 in Table 3 to the density of solution Gradient 3 in Table 3. In a further embodiment, the density of a lipid raft is about the density of solution Gradient 3 in Table 3. In one embodiment, lipid raft is collected based on its density, for example from about 1.00 g/mL to about 1.32 g/mL, optionally from about 1.06 g/mL to about 1.31 g/mL, or about 1.06 g/mL to about 1.30 g/mL, or about 1.06 g/mL to about 1.29 g/mL, or about 1.06 g/mL to about 1.28 g/mL, or about 1.06 g/mL to about 1.27 g/mL, or about 1.06 g/mL to about 1.26 g/mL, or about 1.06 g/mL to about 1.25 g/mL, or about 1.06 g/mL to about 1.24 g/mL, or about 1.06 g/mL to about 1.23 g/mL, or about 1.06 g/mL to about 1.22 g/mL, or about 1.06 g/mL to about 1.21 g/mL, or about 1.06 g/mL to about 1.20 g/mL, or about 1.06 g/mL to about 1.19 g/mL, or about 1.06 g/mL to about 1.18 g/mL, or about 1.06 g/mL to about 1.17 g/mL, or about 1.06 g/mL to about 1.16 g/mL, or about 1.06 g/mL to about 1.15 g/mL, or about 1.06 g/mL to about 1.14 g/mL, or about 1.06 g/mL to about 1.13 g/mL, or about 1.06 g/mL to about 1.12 g/mL, or about 1.06 g/mL to about 1.11 g/mL, or about 1.06 g/mL to about 1.10 g/mL, or about 1.06 g/mL to about 1.09 g/mL, or about 1.06 g/mL to about 1.08 g/mL, or about 1.06 g/mL to about 1.07 g/mL, or about 1.07 g/mL to about 1.31 g/mL, or about 1.07 g/mL to about 1.30 g/mL, or about 1.07 g/mL to about 1.29 g/mL, or about 1.07 g/mL to about 1.28 g/mL, or about 1.07 g/mL to about 1.27 g/mL, or about 1.07 g/mL to about 1.26 g/mL, or about 1.07 g/mL to about 1.25 g/mL, or about 1.07 g/mL to about 1.24 g/mL, or about 1.07 g/mL to about 1.23 g/mL, or about 1.07 g/mL to about 1.22 g/mL, or about 1.07 g/mL to about 1.21 g/mL, or about 1.07 g/mL to about 1.20 g/mL, or about 1.07 g/mL to about 1.19 g/mL, or about 1.07 g/mL to about 1.18 g/mL, or about 1.07 g/mL to about 1.17 g/mL, or about 1.07 g/mL to about 1.16 g/mL, or about 1.07 g/mL to about 1.15 g/mL, or about 1.07 g/mL to about 1.14 g/mL, or about 1.07 g/mL to about 1.13 g/mL, or about 1.07 g/mL to about 1.12 g/mL, or about 1.07 g/mL to about 1.11 g/mL, or about 1.07 g/mL to about 1.10 g/mL, or about 1.07 g/mL to about 1.09 g/mL, or about 1.07 g/mL to about 1.08 g/mL, or about 1.08 g/mL to about 1.31 g/mL, or about 1.08 g/mL to about 1.30 g/mL, or about 1.08 g/mL to about 1.29 g/mL, or about 1.08 g/mL to about 1.28 g/mL, or about 1.08 g/mL to about 1.27 g/mL, or about 1.08 g/mL to about 1.26 g/mL, or about 1.08 g/mL to about 1.25 g/mL, or about 1.08 g/mL to about 1.24 g/mL, or about 1.08 g/mL to about 1.23 g/mL, or about 1.08 g/mL to about 1.22 g/mL, or about 1.08 g/mL to about 1.21 g/mL, or about 1.08 g/mL to about 1.20 g/mL, or about 1.08 g/mL to about 1.19 g/mL, or about 1.08 g/mL to about 1.18 g/mL, or about 1.08 g/mL to about 1.17 g/mL, or about 1.08 g/mL to about 1.16 g/mL, or about 1.08 g/mL to about 1.15 g/mL, or about 1.08 g/mL to about 1.14 g/mL, or about 1.08 g/mL to about 1.13 g/mL, or about 1.08 g/mL to about 1.12 g/mL, or about 1.08 g/mL to about 1.11 g/mL, or about 1.08 g/mL to about 1.10 g/mL, or about 1.08 g/mL to about 1.09 g/mL, or about 1.09 g/mL to about 1.31 g/mL, or about 1.09 g/mL to about 1.30 g/mL, or about 1.09 g/mL to about 1.29 g/mL, or about 1.09 g/mL to about 1.28 g/mL, or about 1.09 g/mL to about 1.27 g/mL, or about 1.09 g/mL to about 1.26 g/mL, or about 1.09 g/mL to about 1.25 g/mL, or about 1.09 g/mL to about 1.24 g/mL, or about 1.09 g/mL to about 1.23 g/mL, or about 1.09 g/mL to about 1.22 g/mL, or about 1.09 g/mL to about 1.21 g/mL, or about 1.09 g/mL to about 1.20 g/mL, or about 1.09 g/mL to about 1.19 g/mL, or about 1.09 g/mL to about 1.18 g/mL, or about 1.09 g/mL to about 1.17 g/mL, or about 1.09 g/mL to about 1.16 g/mL, or about 1.09 g/mL to about 1.15 g/mL, or about 1.09 g/mL to about 1.14 g/mL, or about 1.09 g/mL to about 1.13 g/mL, or about 1.09 g/mL to about 1.12 g/mL, or about 1.09 g/mL to about 1.11 g/mL, or about 1.09 g/mL to about 1.10 g/mL, or about 1.10 g/mL to about 1.31 g/mL, or about 1.10 g/mL to about 1.30 g/mL, or about 1.10 g/mL to about 1.29 g/mL, or about 1.10 g/mL to about 1.28 g/mL, or about 1.10 g/mL to about 1.27 g/mL, or about 1.10 g/mL to about 1.26 g/mL, or about 1.10 g/mL to about 1.25 g/mL, or about 1.10 g/mL to about 1.24 g/mL, or about 1.10 g/mL to about 1.23 g/mL, or about 1.10 g/mL to about 1.22 g/mL, or about 1.10 g/mL to about 1.21 g/mL, or about 1.10 g/mL to about 1.20 g/mL, or about 1.10 g/mL to about 1.19 g/mL, or about 1.10 g/mL to about 1.18 g/mL, or about 1.10 g/mL to about 1.17 g/mL, or about 1.10 g/mL to about 1.16 g/mL, or about 1.10 g/mL to about 1.15 g/mL, or about 1.10 g/mL to about 1.14 g/mL, or about 1.10 g/mL to about 1.13 g/mL, or about 1.10 g/mL to about 1.12 g/mL, or about 1.10 g/mL to about 1.11 g/mL.

In another embodiment, the method further comprises lysing the cells using a lysis buffer at pH of 6.5 comprising 50 mM MES, 150 mM NaCl, 0.5% Triton-X-100 and protease inhibitor cocktail. In a further embodiment, the cells are incubated in the lysis buffer for 30 minutes on ice.

In one embodiment, the loading efficacy is about 1 μg to about 10 μg of cargo protein per 10⁶ EMN. In a further embodiment, the loading efficacy is one of the following: about 1 μg to about 9 μg, about 1 μg to about 8 μg, about 1 μg to about 7 μg, about 1 μg to about 6 μg, about 1 μg to about 5 μg, about 1 μg to about 4 μg, about 1 μg to about 3 μg, about 1 μg to about 2 μg, about 1.0 μg to about 1.5 μg, about 1 μg, about 1.1 μg, about 1.2 μg, about 1.3 μg, about 1.4 μg, about 1.5 μg, about 1.6 μg, about 1.7 μg, about 1.8 μg, about 1.9 μg, about 2 μg, about 2.5 μg, about 3 μg, about 4 μg, about 5 μg, of cargo protein per 10⁶ EMN. In yet a further embodiment, the loading efficacy is about 1.2 μg or about 1.2 μg of cargo protein per 10⁶ EMN. In one embodiment, the cargo is selected from cell-derived medium, BSA, biotin or any other protein having a molecular size similar to a BSA and/or a biotin. In one embodiment, the EMN comprises cell-derived lipid rafts and/or cell-derived plasma membrane.

In one embodiment, the loading efficacy is about 0.1 mg to about 10 mg of cargo protein per 5×10⁸ EMNs. In a further embodiment, the loading efficacy is one or the following: about 0.2 mg, or about 0.3 mg, or about 0.4 mg, or about 0.5 mg, or about 0.6 mg, or about 0.7 mg, or about 0.8 mg, or about 0.9 mg, or about 1 mg, or about 2 mg, or about 3 mg, or about 4 mg, or about 5 mg, or about 6 mg, or about 7 mg, or about 8 mg, or about 9 mg of cargo protein per 5×10⁸ EMNs. In one embodiment, the cargo is a polynucleotide, such as an miRNA. In a further embodiment, the cargo is loaded on a core, such as PLGA. In one embodiment, the EMN comprises cell-derived lipid rafts and/or cell-derived plasma membrane.

In one embodiment, the loading efficacy is about 1×10⁸ to about 1×10¹⁰ copies of cargo polynucleotide per 10⁶ EMNs. In a further embodiment, the loading efficacy is one or more of the following: about 1×10⁹ to about 2×10⁹, or about 1×10⁹ to about 3×10⁹, or about 1×10⁹ to about 4×10⁹, or about 1×10⁹ to about 5×10⁹, or about 1×10⁹ to about 6×10⁹, or about 1×10⁹ to about 7×10⁹, or about 1×10⁹ to about 8×10⁹, or about 1×10⁹ to about 9×10⁹, or about 2×10⁹ to about 3×10⁹, or about 2×10⁹ to about 4×10⁹, or about 2×10⁹ to about 5×10⁹, or about 2×10⁹ to about 6×10⁹, or about 2×10⁹ to about 7×10⁹, or about 2×10⁹ to about 8×10⁹, or about 2×10⁹ to about 9×10⁹, or about 2×10⁹ to about 1×10¹⁰, or about 3×10⁹ to about 4×10⁹, or about 3×10⁹ to about 5×10⁹, or about 3×10⁹ to about 6×10⁹, or about 3×10⁹ to about 7×10⁹, or about 3×10⁹ to about 8×10⁹, or about 3×10⁹ to about 9×10⁹, or about 3×10⁹ to about 1×10¹⁰ copies of cargo polynucleotide per 10⁶ EMNs. In yet a further embodiment, the loading efficacy is one or more of the following: about 1×10⁹, or about 2×10⁹, or about 2.1×10⁹, or about 2.2×10⁹, or about 2.3×10⁹, or about 2.4×10⁹, or about 2.5×10⁹, or about 2.6×10⁹, or about 2.7×10⁹, or about 2.8×10⁹, or about 2.9×10⁹, or about 3×10⁹, or about 3.1×10⁹, or about 3.2×10⁹, or about 3.3×10⁹, or about 3.4×10⁹, or about 3.5×10⁹, or about 3.6×10⁹, or about 3.7×10⁹, or about 3.8×10⁹, or about 3.9×10⁹, or about 4×10⁹, or about 5×10⁹, or about 6×10⁹, or about 7×10⁹, or about 8×10⁹, or about 9×10⁹, or about 1×10¹⁰ copies of cargo polynucleotide per 10⁶ EMNs. In one embodiment, the loading efficacy is about 3×10⁹ or about 3.3×10⁹ copies of cargo polynucleotide per 10⁶ EMNs. In one embodiment, the cargo is an miRNA. In a further embodiment, the cargo is loaded on a core, such as PLGA. In one embodiment, the EMN comprises cell-derived lipid rafts and/or cell-derived plasma membrane.

In certain embodiments, the yield is more than about 1×10⁸ EMNs per mL, for example, from about 3.46×10⁸ to about 6.33×10⁸EMNs per mL. In certain embodiments, the yield is more than about 1×10⁸ (for example, more than about 2×10⁸, or more than about 3×10⁸, or more than about 4×10⁸, or more than about 5×10⁸, or more than about 6×10⁸, or more than about 7×10⁸, or more than about 8×10⁸, or more than about 9×10⁸, or more than about 1×10⁹, or more than about 2×10⁹, or more than about 3×10⁹) EMNs per 10⁷ cells from which the lipid rafts/plasma membrane is derived, for example about 3.78×10⁹ EMNs per 10⁷ cells from which the lipid rafts/plasma membrane is derived.

In certain embodiments, the yield is more than about 1×10⁸, or more than about 2×10⁸, or more than about 3×10⁸, or more than about 4×10⁸, or more than about 5×10⁸, or more than about 6×10⁸, or more than about 7×10⁸, or more than about 8×10⁸, or more than about 9×10⁸, or more than about 1×10⁹, or more than about 2×10⁹, or more than about 2.5×10⁹ EMNs (i.e., EMN particles or particles)/mL for 1 mg of PLGA. In one embodiment, the yield is about 2.53×10⁹ EMNs (i.e., EMN particles or particles)/mL for 1 mg of PLGA, and further optionally wherein the method is scalable.

In one embodiment, the cells are cultured in a bioreactor.

In any embodiment and/or aspect relating to a cell, the cell may be a differentiated cell or a stem cell. In one embodiment, the cell is selected from the group of an endothelial cell, a cardiomyocyte, a myogenic cell, a smooth muscle cell, a neuron, an astrocyte, an oligodendrocyte, an olfactory ensheathing cell, a microglial cell, a tumor cell, a cancer cell, an immune cell, a neutrophil, an eosinophil, a basophil, a mast cell, a monocyte, a macrophage, a dendritic cell, a natural killer cell, a lymphocyte, a B cell or a T cell. Additionally or alternatively, the cell is an animal cell, a mammalian cell or a human cell. In certain embodiments, the stem cell is an adult stem cell and/or an embryonic stem cell. In a further embodiment, the stem cell is selected from a neuronal stem cell, an endothelial progenitor cell (EPC), a cord-blood derived EPC, a umbilical cord-derived EPCs, a mesenchymal stem cell, an adipose derived stem cell, a bone marrow derived stem cell, a placental-derived MSC (PMSC), or an induced pluripotent stem cell (iPSC). In yet a further embodiment, the mesenchymal stem cell expresses one or more of CD105⁺, CD90⁺, CD73⁺, CD44⁺ and CD29⁺ and CD184+. Additionally or alternatively, the mesenchymal stem cell lacks one or more of hematopoietic markers. In a further embodiment, the hematopoietic markers are selected from the group of: CD31, CD34 and CD45. In certain embodiments, the stem cell is a mesenchymal stem cell that expresses one or more exosome specific markers selected from the group of CD9, CD63, ALIZ, TSG101, alpha 4 integrin, beta 1 integrin, and/or the stem cell is a mesenchymal stem cell lacks expression of calnexin. In one embodiment, a human stem cell. In certain embodiments, the stem cell is isolated from a pediatric, fetal, early-gestation or pre-term placenta-derived stem cell. In one embodiment, the cell is an apoptotic cell. In another embodiment, the neuron is an isolated cortical neuron or a spinal cord neuron.

Further provided is a kit comprising an EMN and/or a composition as described herein, and optionally, reagents and instructions for use of one or more diagnostically, as a research tool or therapeutically. In one aspect, provided is a kit comprising an EMN, or a plurality, or a composition as disclosed herein, and instructions for use. In one embodiment, the instructions comprise instruction for carrying a method as disclosed herein.

Also provided are methods to isolate, manufacture, expand, quantify and qualify the EMNs as described herein.

The following examples are provided to illustrate various aspects of this disclosure.

### Experiment No. 1—Human Placenta-Derived Mesenchymal Stromal Cell Exosome-Mimicking Nanovesicles for Neuroprotection

Neurological diseases are prevalent throughout the world populations and drastically affect the lives of people of various age groups. Numerous factors contribute to the development of neurological disease, such as, genetic mutations and environmental conditions, infections, congenital abnormalities and injuries to the central nervous system (CNS). Several neurological diseases lead to neurodegeneration that arise from irreversible damage or loss of neurons and the glial cells of the CNS.

Analyses of research data indicate that MSCs have immunomodulatory (Lee et al., Int Immunopharmacol, (2012), neuroprotective (Calzarossa et al., Neuroscience, (2013)) and wound healing properties (Jones et al., PLos One, (2012)). These immunomodulatory and neuroprotective properties of MSCs are associated with paracrine secretions such as unique cytokines (Pashoutan Sarvar et al., Adv Pharm Bull, (2016)), growth factors (Talwadekar et al., Scientific Reports, (2015)) and extracellular vesicles (Gnecchi et al., Methods Mol Biol, (2016); Mirotsou et al., Journal of Molecular and Cellular Cardiology, (2011); Liang et al., Cell Transplant, (2014).

TABLE 1
Delivery of paracrine secretions

| Type of | | |
|---|---|---|
| Carrier | Advantages | Disadvantages |
| Synthetic | Versatile | Need surface modification |
| (Liposomes) | Economical | Unstable |
| Natural | Targeted delivery | Low yield (Li et al., 2018)/ |
| (Exosomes) | Immunomodulatory | loss during purification (Jo, |
| | (Pashoutan Sarvar et al., | Kim et al., 2014) |
| | 2016) | |

Applicant's laboratory has extensively demonstrated that human placenta-derived MSCs (hPMSCs) when used to treat fetal lamb myelomeningocele lambs in utero, significantly improved the ambulatory functions of the lambs (Wang et al., Stem Cells Transl Med, (2015); Lee et al., Int Immunopharmacol, (2012)). Previous studies from Applicant's lab have shown that hPMSCs secrete significant levels of paracrine factors such as hepatocyte growth factor (HGF), brain-derived neurotrophic factor (BDNF), vascular endothelial growth factor (VEGF) (Lankford et al., World Jurnal of Stem Cells, (2015)) and several cytokines (Wang et al., Stem Cells Transl Med, (2015) that help ameliorate neuronal damage). BDNF is responsible for neuronal damage repair, VEGF is responsible for the formation of blood vessels and oligodendrocytes, and HGF is associated with neuroprotective functions (Bai et al., Nat Neurosci, (2012)). Although MSC transplantation is a potential treatment option for neurological diseases, the administration of these cells could result in graft rejection and limited or unintended engraftment(Gnecchi et al., Methods Mol Biol, 2016; Gnecchi et al., Nat Med, (2005); Lou et al., Exp Mol Med, (2017)). To overcome these drawbacks, further studies were focused on characterizing and understanding the role of MSC secretome.

Among the components of the MSC secretome, exosomes are currently studied as a potential cell-free therapy. Exosomes are double-layered extracellular vesicles, 50-150 nm in diameter, secreted by various types of cells such as neurons, stem cells, B and T lymphocytes, dendritic cells, mast cells, platelets and adipocytes (Guo et al., Neuropsychiatr Dis Treat, (2017); Lee et al., Int Immunopharmacol, (2012); van der Pol et al., Pharmacol Rev, (2012)). Since the biogenesis of exosomes involves the invagination of the plasma membrane predominantly at the lipid raft domains, the exosomes retain the composition and cell-specific markers of the plasma membrane (Pike, Journal of Lipid Research, (2003); Lingwood et al., Science, (2010); de Gassart et al., Blood, (2003)). Their small size, cell membrane composition and immunomodulatory functions have made exosomes ideal nanocarriers for drug delivery(Tran et al., Clin Immunol, (2015)).

Recent in vitro studies have shown that hPMSC-derived exosomes are neuroprotective but their yield from conventional cell culture production is significantly low (Jo et al., Nanoscale, (2014)). Furthermore, their effective purification is often difficult due to exosome loss during processing, thus requiring a large number of cells to obtain a therapeutically significant and clinically relevant number of exosomes. Moreover, recent research has shown that the composition and contents of native exosomes are variable, heterogeneous (Brenner et al., Methods Mol Biol, (2019)). The cargo of exosomes depend on the type, metabolic state and environmental stress of the donor cell from which the exosomes were isolated (Jelonek et al., Protein and peptide letters, (2016)). In order to overcome the above issues, Applicant proposed to synthesize stem cell exosome-mimicking nanovesicles (EMNs) by using hPMSC-derived lipid rafts and encapsulating the hPMSC secretome devoid of native exosomes.

Lipid rafts, the highly ordered sections within the plasma membrane, are composed of glycosphingolipids and cholesterol that play an important role in cell adhesion, migration, transport and signal transduction (Pike, Journal of Lipid Research, (2003)). In addition to their similarity to exosomes outer membrane, lipid rafts possess several properties such as a dynamic structure that helps in assembly and cell-surface receptors that assist in cellular uptake (Varshney et al., Immunology, (2016); Alonso et al., Journal of Cell Science, (2001)). Due to these advantages and without wishing to be bound by the theory, Applicant hypothesized that the hPMSC-derived lipid rafts will allow for the encapsulation of hPMSC secretome (i.e. conditioned medium devoid of native exosomes) and will be able to interact with the target cells and effectively deliver hPMSC paracrine secretions. In addition, Applicant hypothesized that the hPMSC secretome containing neuroprotective factors encapsulated within the EMNs, will exhibit the therapeutic potential to rescue apoptotic neurons in culture.

In one aspect, this invention focuses on the synthesis of stem cell derived exosome-mimicking nanovesicles (EMNs) that are similar to native exosomes in size, composition and biological function. The synthesis of EMNs involved encapsulating concentrated exosome-free conditioned medium into PMSC-derived lipid rafts. Without being bound by theory, it was hypothesized that the PMSC-derived EMNs would have the therapeutic potential to rescue apoptotic neurons in culture. The results of this study indicated that the EMNs were successfully loaded with PMSC secretions and formed spherical vesicles with a size range of 50-200 nm. A total of 3.78×10⁹ EMNs were produced from 10 million cells, thus overcoming the low yields of collection seen with native exosomes. Additionally, the EMNs could rescue the neurons that were undergoing apoptosis when compared PBS-treated neurons, thus corroborating the fact that they are not only similar to native exosomes in terms of their size and membrane composition, but are also able to function similar to native exosomes.

The data reported herein that using PMSC-derived lipid rafts to produce exosome-mimicking nanovesicles to deliver neuroprotective secretome, addressing the need for an effective system to facilitate the delivery of stem cell paracrine secretions and neuroprotective agents in a scalable manner.

Through this research, Applicant devised a technique to isolate lipid rafts from the cell membrane of hPMSCs. Applicant's results indicate that lipid rafts collected from the detergent-resistant fraction of hPMSCs express exosome-specific markers such as CD9, CD63, ALIX, TSG101 and integrin such as α4 and β1. The lipid rafts were extruded through filters of varying pore sizes to form EMNs. The EMNs successfully encapsulated fluorescein isothiocyanate-labelled bovine serum albumin (FITC-BSA) and biotin (FITC-Biotin). Optimal nanovesicle loading was obtained when lipid raft vesicles were loaded with 0.5 mg/mL FITC-BSA and FITC-Biotin. Further, the EMNs that were loaded with 0.5 mg/mL concentrated conditioned medium and imaged using transmission electron microscopy displayed structure and shape similar to that of exosomes. The production of EMNs could be scaled up to produce 3.78×10⁹ vesicles from 10 million hPMSCs. Addition of the conditioned medium loaded EMNs to neurons undergoing apoptosis in vitro indicated that the EMNs could also rescue apoptotic neurons.

hPMSC Culture

1×10⁶ hPMSCs were cultured in T150 tissue culture treated flask with D5 media containing Dulbecco's modified eagle's medium (DMEM) with high glucose, 5% fetal bovine serum (FBS), 20 ng/mL fibroblast growth factor (FGF) and 20 ng/mL epithelial growth factor (EGF) at 37° C., 5% CO₂ for 7 days until they reached 90% confluence and are between 6-7×10⁶ cells. The cells were washed with 10 mL phosphate-buffered saline (PBS) and lifted off using 6 mL of TrypLe, neutralized with 18 mL DMEM and centrifuged at 470×g until the cells pelleted at the bottom. The pellet was re-suspended in 5 mL D5 media, 10 μL of the suspension was mixed with 10 μL Trypan Blue and counted using trypan blue exclusion method. 4000 cells/cm² were seeded on six 150 mm dishes with 15 mL of media and cultured at 5% CO₂ and 37° C. for 7 days until the plates were 95% confluent.

### Isolation of Lipid Rafts

As the composition of lipid rafts is mainly lipid, they can be effectively separated on a hydrophilic sucrose gradient. The increased presence of lipids and proteins within the rafts makes them float to the low-density regions of the sucrose gradient, thus they are commonly found as a band between the 5 and 30%.

In one aspect, seven 150-mm dishes with 90-95% confluent hPMSCs were washed with 7 mL of ice cold PBS (4° C.). Then 7 mL of ice cold PBS was added to the dishes and the cells were gently scraped using a cell scraper and the supernatant was collected to a 50 mL conical centrifuge tube. The plates were washed with 3 mL of fresh ice cold PBS and was added to the 50 mL conical centrifuge tube. The cell suspension was centrifuged at 470×g in the Sorvall RT 6000D centrifuge. The pellet was re-suspended in 5 mL ice cold PBS and the cells were pooled into one 50 mL conical centrifuge tube. The cells were counted as described earlier and 20-25×10⁶ cells were pelleted down and resuspended in 2 mL of lysis buffer (pH of 6.5; Table 2) containing 50 mM MES, 150 mM NaCl, 0.5% Triton-X-100 and protease inhibitor cocktail and incubated on ice for 30 minutes. Following the lysis, 378 μL of the cell lysate was mixed with a 522 μL of 60% OptiPrep™ to obtain a final concentration of 35% OptiPrep™ gradient and added to the bottom of a 5 mL Beckman Coulter ultracentrifuge tube. Then 900 μL of Optiprep™ gradients were sequentially added in the following order: 30%, 25%, 20% and 0% (Table 3). Care was taken not to mix the gradients while adding. The samples were centrifuged at 200,000×g at 4° C. for 4 hours using a SW 55 rotor and Beckman L7 ultracentrifuge. After centrifugation, the gradients from three ultracentrifuge tubes were collected in 500 μL fractions starting from the top to bottom and transferred to nine 1.5 mL Eppendorf tubes—these tubes were subjected to dot-blot analysis. In the remaining three ultra-centrifuge tubes, the lipid rafts viewed as a ring between 20-30% gradient were collected and transferred to a new ultracentrifuge tube. 4 mL of PBS was added to the tubes with the lipid rafts and centrifuged at 200,000×g for 40 minutes. The supernatant was aspirated and 1 mL of fresh PBS was added. The addition of PBS caused the lipid rafts to float up like a thin film and the Eppendorf tube containing the floating lipid raft was stored at −80° C.

TABLE 2
The composition and preparation of lysis buffer and MBS buffer

| | Volume (mL) |
|---|---|
| Lysis buffer (50 mM MES, pH 6.5, | |
| 150 mM NaCl, 0.5% Triton-X-100) | |
| 0.5 MMES stock, pH 6.5 | 2 |
| 1M NaCl stock | 3 |
| 10% Triton-X-100 stock | 1 |
| Milli-Q H₂O | 14 |
| Total | 20 |
| MBS buffer | |
| 0.5M MES stock, pH 6.5 | 1 |
| 1M NaCl stock | 1.5 |
| Milli-Q H₂O | 7.5 |

TABLE 3
The preparation of OptiPrepTM gradients

| | OptiPrep ™ | Cell | MBS | OptiPrep ™ | Total |
|---|---|---|---|---|---|
| | percentage | lysate | buffer | solution | volume |
| Gradient | (%) | (μL) | (μL) | (μL) | (μL) |
| 1 (bottom) | 35 | 378 | 0 | 522 | 900 |
| 2 | 30 | — | 500 | 500 | 1000 |
| 3 | 25 | — | 580 | 420 | 1000 |
| 4 | 20 | — | 650 | 350 | 1000 |
| 5 (top) | 0 | — | 1000 | — | 1000 |

Note: The final volume is 1000 μL out of which 900 μL is added to the ultracentrifuge tube for gradient preparation. 10 μL of protease inhibitor cocktail was added to all the 1000 μL gradients prior to use.

In another aspect, hPMSCs are cultured, at 37° C. and 5% CO₂, until they are 80% confluent. Cells are pelleted, lysed and subjected to sucrose gradient centrifugation. The sucrose gradients are 80%, 30% and 5% and centrifuged at 270000×g for 16 h to obtain lipid rafts situated between the 5% and 30% gradient. Lipid rafts are characterized by assessing the presence of raft-specific markers such as flotillin 1, caveolin 1, cell membrane-specific markers such as integrins and Annexins and exosome markers such CD 9/63/81, Alix and TSG101 by Western blotting. Flotillin-1 and caveolin-1 ensure successful raft isolation as they are found on both leaflets of the rafts. CD 9/63/81, Alix and TSG101 are markers of exosomes. See for example, Gupta et al. (2014)

Detection of Lipid Rafts within the Gradient

The Bio-Rad dot blot apparatus was set up according to the manufacturer's instruction. The nitrocellulose membrane was rinsed with Tris-buffered saline (TBST) containing 20 mM Tris base, 500 mM NaCl and 0.5% Tween-20 at pH 7.5. 200 μL of the fractions collected during lipid raft isolation was loaded into each well of the 96-well dot blot apparatus (at airflow setting). Gravity flow setting allowed the entire sample to filter through the membrane. 200 μL of 1% bovine serum albumin (BSA) in TBST was added to each of the wells and allowed to filter through the membrane by gravity. Once the BSA was filtered completely, the apparatus was switch to the vacuum flow and 200 μL of TBST was added to wash the membrane for three sequential washes. Following the washes, the apparatus was switched to airflow and 100 μL of caveolin-1 at the concentration 1:1000 was added to each of the wells and allowed to filter down completely for 1 hour. When the primary antibody had not drained completely, vacuum was applied for 90 seconds to ensure complete drainage. The membrane was washed with 200 μL of TBST for 3 washes and 200 μL of Anti-Rabbit HRP secondary antibody at 1:2500 was added under the airflow setting and allowed to drain slowly for 1 hour. The nitrocellulose was washed with TBST under vacuum for 3 times as previously described. The nitrocellulose was removed and probed with 1.5 mL of a 1:1 mixture of luminol enhancer and peroxide buffer of the Super Signal West Dura kit. After a 5 minute incubation, the membrane was imaged using the Bio-Rad ChemiDoc XRS+ System enabled with the Image Lab software.

### Characterization of Lipid Rafts

The lipid raft pellet was re-suspended in 1 mL of sterile PBS. 16.25 μL of the lipid raft sample was mixed with 6.25 μL of NuUPAGE LDS sample loading buffer and 2.5 μL of 10× Dithiothreitol (DTT). A non-reducing sample was prepared without DTT. The reducing sample and the non-reducing sample were incubated at 70° C. for 10 minutes and centrifuged at 16,000×g for 2 minutes. The SDS-PAGE gel apparatus was set up according to the manufacturer's instructions, 8 μL of Novex protein standard and 20 μL of samples were added to the wells. The SDS-PAGE gel was allowed to run at a constant voltage of 150 V until the dye front reached the bottom edge of the gel support. After the completion of the run, the gel was washed with 10 mL of transfer buffer (0.025 M Tris-base, 0.19 M glycine and 20% methanol) and assembled into the electro-blotting sandwich. The proteins were transferred to nitrocellulose membrane at a constant voltage of 100 V for 45 minutes. After the transfer, the nitrocellulose membrane was stained with Ponceau stain for 5 minutes (to visualize the transfer and to enable to cut the lanes), followed with multiple washes of MlliQ water and blocked with 5% non-fat dry milk for 1 hour. The membrane was washed 3 times with TBST and each lane was individually probed with 1:500 dilution of ALIX, TSG101, integrin α4 and β1, Calnexin and 1:1000 dilution of Caveolin-1 and Flotillin-1 overnight at 4° C. on a rocker. The following day, the nitrocellulose was washed 4 times with TBST with gentle rocking for 10 minutes for every wash. The membrane was probed with 1:2500 dilution of anti-rabbit HRP antibody for 1 hour at room temperature. After the secondary antibody incubation, the nitrocellulose membrane was washed 4 times with TBST and probed with the chemiluminiscence substrate and imaged in the Bio-Rad ChemiDoc XRS+ System enabled with the Image Lab software.

### Loading Efficiency Determination

Loading efficiency is the capacity of the raft vesicles to hold a cargo, for example the proteins of interest. Used herein is a fluorescein isothiocyanate-labelled bovine serum albumin (FITC-BSA). Following is a comparison of protein sizes indicating bovine serum albumin (BSA) can serve as a suitable representation of the proteins/peptide contained in the conditioned medium: BDNF (14 kDa)<VEGF (27 kDa)<BSA (66.5 kDa)<HGF (83.1 kDa)

In one aspect, the isolated lipid rafts are mixed with FITC-BSA at varying concentrations and extruded using a Mini Extruder with filters of decreasing pore size from 10 μm to 100 nm to form EMNs. The morphology and size distribution of the FITC-BSA containing EMNs are measured using TEM and NTA, respectively. In addition, the concentration of fluorescent protein within the EMN are measured using a microplate reader. Further, using the above data the uptake efficiency are calculated to determine the amount of conditioned media to be used for EMN synthesis.

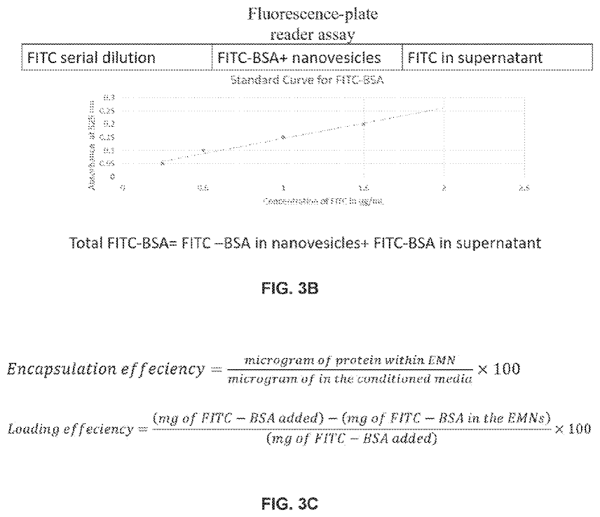

In a further aspect, a lipid raft pellet was re-suspended in 1 mL fluorescein isothiocyanate bovine serum albumin (FITC-BSA) and FITC-Biotin solutions at concentrations 0.25, 0.5 and 1 mg/mL, respectively, and extruded through a mini-extruder according to the manufacturer's instructions (FIG. 3). The lipid raft-FITC-BSA/FITC-Biotin samples were extruded successively 30 times through each membrane of pore sizes 400 nm, 200 nm and 100 nm. After extrusion the sample was collected and stored in black centrifuge tubes to prevent the loss of fluorescence. 50 μL of the FITC-BSA loaded vesicles were filtered through a Pierce BSA depletion column according to the manufacturer's instruction. The FITC-Biotin loaded vesicles were spun down at 16,000×g for 10 minutes. The supernatant was collected and the vesicles at the bottom were washed with 500 μL of PBS and re-centrifuged at 16,000×g for a total of 5 washes. The FITC-BSA and FITC-Biotin vesicles were read in Nanodrop™ 2000 after blanking with unloaded vesicles extruded with water. The absorbance of FITC-BSA and Biotin was measured before loading and the absorbance of the loaded vesicle was subtracted from the initial value to obtain the loading efficiency of the sample. The loading efficiency was highest at a concentration of 0.5 mg/mL and this was fixed as a loading concentration for conditioned medium-loaded nanovesicles.

In one embodiment, the max loading was about 0.6 mg of cargo protein (for example, Biotin) in 4.896×10⁸ EMNs which is 1.22 microgram (μg) per 10\hat{ }6 particles.

### Conditioned Medium Collection and Concentration

In one aspect, PMSCs were seeded on to 150 mm tissue culture treated dishes at 100,000 cells/cm² in 20 mL D5 media and cultured at 5% CO₂ and 37° C. for 48 hours. After 48 hours, the conditioned medium was collected and spun down at 470×g to remove cell debris. The supernatant was transferred to a clean ultracentrifuge tube and centrifuged at 112,600×g in SW 28 rotor for 90 min to deplete native exosomes. The supernatant was then concentrated by centrifuging through an Amicon Ultra-15 centrifugal 3 kDa filter unit for 90 minutes until the conditioned medium was concentrated to 20 times. The BSA present in the concentrated conditioned medium was removed by using the HiTrap™ Blue HP albumin depletion kit, according to the manufacturer's instructions. The D5 media, concentrated conditioned medium before BSA depletion, BSA depleted medium, the albumin entrapped in the column and albumin standard were loaded onto a 4-12% Bis-Tris NuPAGE gel and stained using Imperial™ protein stain to determine the effect of BSA depletion.

In another aspect, PMSCs are seeded at 20,000 cells/cm² for T₁₅₀ flask with exosome-depleted FBS containing D5 media for 48 h at 5% CO₂ at 37° C. Condition medium is then collected by centrifuging at 1500×g for 20 min. Media is concentrated using Amicon Ultra-15 centrifugal filter units with a 3 kDa molecular weight cutoff and stored at −80° C. until use.

### Synthesis of EMNs and Nanoparticle Tracking Analysis

In one aspect, the exosome-depleted conditioned media obtained from hPMSCs is concentrated and subjected to ELISA to detect the presence of BDNF, HGF and VEGF. The lipid rafts are mixed with the varying concentrations of conditioned media and extruded through a Mini Extruder to form EMNs containing the conditioned media. Following synthesis, the morphology of EMNs is measured using TEM and the size distribution and concentration of EMNs is analyzed by NTA. Since neuronal damage, via apoptosis, is a common occurrence during the progression of neurological diseases, the neuroprotective ability of EMNs is assessed by using established methods. Subsequently, the neurites are assessed for branching points, circuitry length and segments by using WimNeuron Analysis (Wimasis).

In a further aspect, the lipid raft pellet was resuspended in the concentrated conditioned medium and extruded using the Mini Extruder with polycarbonate filters of reducing pore size (400-100 nm). The formed EMNs were concentrated by centrifuging at 16000×g and the EMNs pelleted in the bottom 50 μL fraction were collected. The EMNs were subjected to nanoparticle tracking analysis to obtain the concentration and size distribution. 50 μL of the EMNs sample was added to 950 μL of 0.22 μm triple-filtered water and loaded on to the stage of the Nano Sight LM10 with a 404-nm laser and imaged using the sCMOS camera provided with the instrument. Using the NTA software v 3.0 software, three 90-second videos captured at a screen gain of 10, detection threshold of 3 and camera level of 12 were analyzed to determine the size and concentration of the EMNs.

NTA is a technique used to characterize the number and size distribution of nanovesicles (Nano Sight LM10). In one embodiment, the sample was diluted with triple-filtered (0.2 μm) MilliQ-water (MQ-H₂O) to reach a concentration of 3-20×10⁸ particles/mL. 90 second videos were recorded and analyzed using NTA 3.0 software. See, Kumar et al. (2019)

### Exosome Collection (Control)

PMSCs are seeded at 20,000 cells/cm² in for T₁₅₀ flask with exosome-depleted FBS containing D5 media for 48 h at 5% CO₂ at 37° C. The media is then centrifuged at 300×g for 10 min, 2000×g for 20 min and passed through 0.2 μm filter. The media is also concentrated using Amicon Ultra-15 centrifugal filter units with a 100 kDa filter. After being transferred to a thick wall polypropylene tube and centrifuging at 8836×g, the following steps are performed once or repeated: the supernatant is then further centrifuged at 112,700×g for 90 min and the pellet is resuspended in PBS (this supernatant is conditioned media free of exosomes).

### Transmission Electron Microscopy

In one aspect, the surface morphology of the EMNs was studied using an established negative protocol for characterizing exosomes (Thery et al., 2006). 50 μL of the conditioned medium loaded-EMNs was mixed with equal volume of 4% paraformaldehyde and 5 μL of this mixture was added on to three Formvar-carbon coated electron microscopy (EM) grids each. The grids were washed with a Parafilm strip containing 100 μL PBS by gently touching the grid o the drop edge with the help of a pair of forceps. The grid was touched to 50-μL, drop of 1% glutaraldehyde and incubated for 5 minutes following which the grids were washed for 8 times with 100 μL of distilled water by allowing the grid to stay immersed in the water for 2 minutes. The grids were then transferred to a 50-μL, drop of uranyl-oxalate (pH, 7.0) for 5 minutes. The grids were transferred to a 50-μL, drop of methyl cellulose UA solution and incubated for 10 minutes on ice. Finally, the sides of the grids were gently tapped against a filter paper and were imaged at 80 V using a CM120 transmission electron microscope.

In another aspect, negative-staining protocol is established. Briefly, the cells are fixed in 2% PFA. 5 μl resuspended pellets is deposited on Formvar-carbon coated EM grids. Two or three grids are prepared for each exosome preparation. The sample is then covered and the membranes were allowed for adsorbing for 20 min in a dry environment. Following fixing and staining of adsorbed exosomes, TEM images are examined using CM120 transmission electron microscope (Philips/FEI BioTwin, Amsterdam, Netherlands) at 80 kV. See, for example, Thery et al., Curr Protoc Cell Biol (2006).

### Functional Assay/Neuroprotection Assay

In one aspect, the neuroprotective ability of the EMNs was investigated by using a neuroprotection model developed and established in Applicant's lab (Kumar et al., (2019)). SH-SY5Y neuroblastoma cells were cultured in D5 media at 37° C. and 5% CO₂ for up to 5 passages. 100,000 SH-SY5Y cells/cm² were seeded on an 8-well Permanox^{R} chamber slides and cultured at 37° C. and 5% CO₂ for 24 hours. Apoptosis was induced by treating the cells with 1 μM staurosporine for 4 hours. The cells were washed with 200 μL of warm (37° C.) D5 media and 1000, 2000, 4000 and 8000 EMNs/cell diluted in 300 μL (37° C.) media were added directly to the apoptotic cells and incubated for 96 hours at 37° C., 5% CO₂. After 96 hours, the cells were washed with 2 mL PBS stained for 2 min using 2 μM Calcein AM. The stained cells were then imaged at 5× magnification using the Carl Zeiss Axio Obeserver D1 to observe for improvement in neuronal survival after apoptosis.

In another aspect, apoptosis of SH-SY5Y (derived from a cell line of neuroblastoma) cells was induced via staurosporine, serving as a model commonly used to analyze neuron function and differentiation. The cells were then treatment with EMNs, and recovery analysis was performed using WimNeuron Analysis (neurite outgrowth, branching and circuitry length). Confirmatory test includes measuring caspase-3 activity that leads to cleavage of its substrate PARP-1, that ultimately leads to fragmentation of DNA that can be assessed by TUNEL staining. GAPDH is used as a control.

### Results

Isolation and Characterization of Lipid Rafts from Human Placental Mesenchymal Stem Cells hPMSCs

Briefly, the lipid rafts from hPMSCs are isolated using sucrose gradient centrifugation. Following isolation, the lipid rafts are characterized for lipid raft-specific, cell-specific and exosome-specific markers. Without wishing to be bound by the theory, the lipid ring located at a certain (for example, between the 5% and 30% or about 20% to about 30%) sucrose gradient consists of lipid rafts, which having a composition similar to that of hPMSC cell membrane.

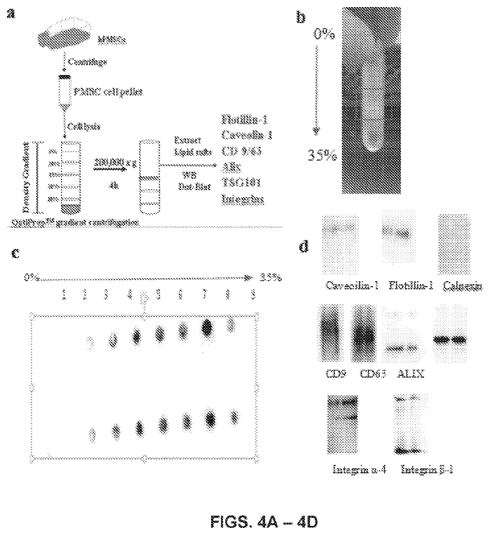

To obtain the lipid rafts the hPMSC cell lysate was subjected to density gradient centrifugation using an OptiPrep™ lysed (FIG. 4A). During ultracentrifugation, the various cell components of the cell lysate fractionate based on their density (FIG. 4A & FIG. 4B). The gradients between 20% and 30% contained a white ring-like structure that contained the lipid rafts. The gradient fractions between 0%-35% gradients (collected as 500 μL aliquots) when assessed by dot blot indicated a positive signal for Caveoilin-1, thus confirming the location of lipid rafts between the 20% and 30% gradient (FIG. 4C). Since the raft-specific markers were detected between 20% and 30% gradients the lipid raft ring at this location was precipitated and probed for exosome-specific markers ALIX, TSG101, CD9 and CD63 and failed to express endoplasmic reticulum marker Calnexin, suggesting that the lipid raft isolation was complete and that the vesicles share some of the markers present on native hPMSC exosomes (FIG. 4D). The presence of Integrin α4 and β1 indicate that the lipid raft vesicles contain cell surface receptors that can assist in targeted delivery similar to that of native exosomes (FIG. 4D).

### Determination of Loading Efficiency

The isolated lipid rafts along with fluorescein isothiocyanate-labelled bovine serum albumin (FITC-BSA) were extruded through the Mini Extruder. The morphological feature was analyzed using transmission-electron microscopy (TEM) and the size distribution and concentration of the EMNs were measured using nanoparticle tracking analysis (NTA). Subsequently, the concentration of the FITC-BSA within the EMNs was measured using a microplate reader to confirm the loading efficiency of these EMNs. Without wishing to be bound by the theory, the lipid rafts undergo structural reorganization to form EMNs encapsulated with FITC-BSA.

The results from the loading show that the EMNs were able to encapsulate the FITC-BSA and FITC-Biotin and that the optimum loading concentration was at 0.5 mg/mL (FIG. 5A). 50 μL of the loaded EMNs were diluted in 950 μL of Triple-filtered water and analyzed using the Nano Sight LM10. The NTA analysis indicated that EMNs had an average size range of 187.62±5.1 nm and concentration of 4.896×10⁸±1.43×10⁸ vesicles/mL (FIG. 5B). TEM imaging showed that the 0.5 mg/mL FITC-BSA loaded EMN had a circular morphology with a smooth edge (FIG. 5C) unlike the cup-shaped structure of native exosomes (Thery et al., 2006).

### Concentrating the Conditioned Medium

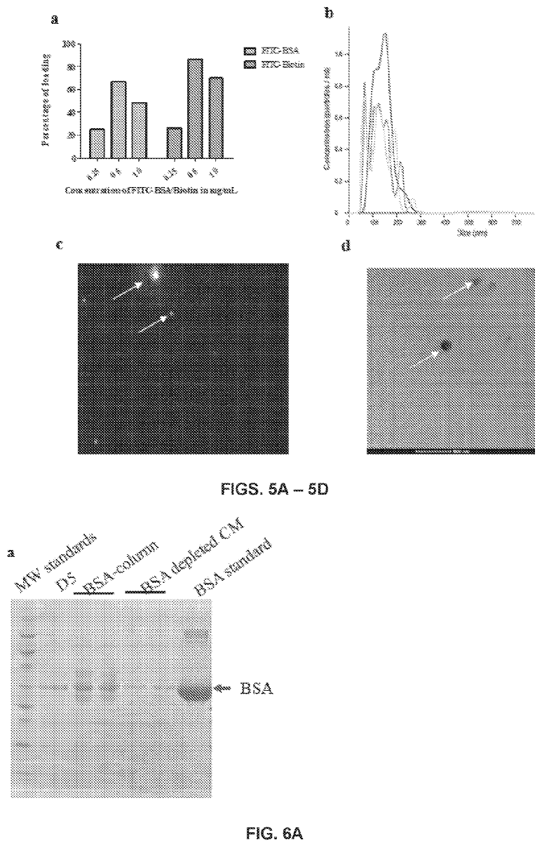

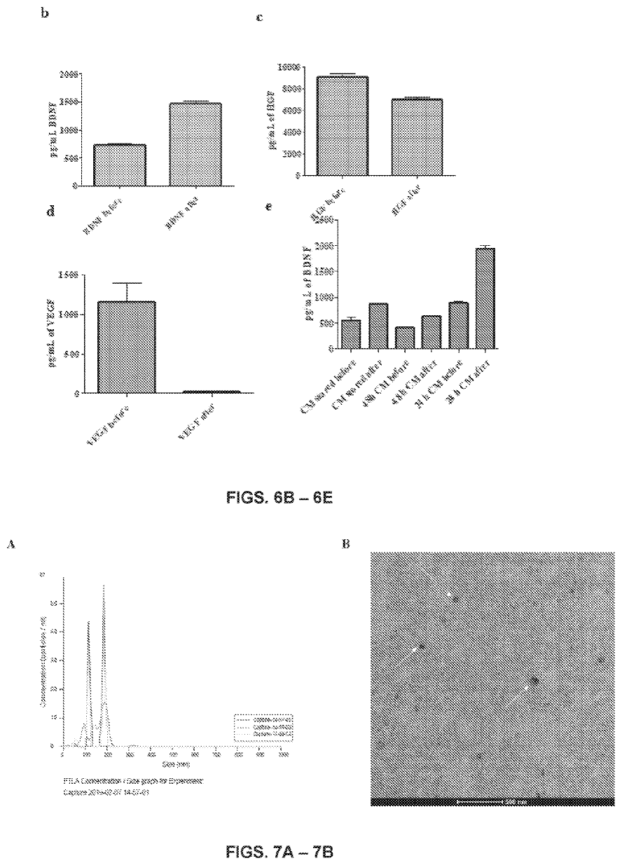

Previous studies from Applicant's lab have shown that the conditioned medium obtained at 24-hour time point is known to contain significant levels of BDNF, HGF and VEGF (Kumar et al., 2019). Since BSA is found in FBS used in the culture medium, the hPMSC secretome was concentrated up to 20 times and subjected to BSA depletion using the HiTrap™ column. Subsequent gel electrophoreses showed that the BSA band 66-kDa band corresponding to BSA was reduced to ⅓ the amount compared to medium control (lane 5 and 6 compared to lane 2; FIG. 6A). The albumin rich fraction of the BSA that was entrapped within the column formed a larger band at 66 kDa compared to the depleted fraction (lane 3 and 4 compared to lane 5 and 6; FIG. 6A). Recent studies in Applicant's lab have shown that the hPMSC secretome contains BDNF, HGF and VEGF that play an important role in neuroprotection (Kumar et al., ((2019). In order to confirm the presence of these growth factors, Applicant analyzed the secretome using enzyme-linked immunosorbent assay (ELISA). The levels of BDNF secreted by hPMSC was 1420.48 pg/mL (FIG. 6B), HGF was 6229.54 pg/mL and VEGF was 1169.65 pg/mL (FIG. 6C & FIG. 6D). The level of BDNF was increased 2 times indicating that the presence of BSA hindered the detection of BDNF. However, the levels of VEGF decreased by 100 folds and HGF decrease by 1.3 folds likely because these growth factors are being bound to the depletion column in a non-specific manner. Since storage affects the stability of proteins, Applicant tested the effects of storage on the levels of BDNF at 24 hours to ensure that the BDNF levels can be normalized to the initial cell seeding density (Polyakova et al., International Journal of Molecular Sciences, 2017). The levels of BDNF was 2 times higher in 48-hour conditioned medium as opposed to the conditioned medium collected 30 days prior (stored at −80° C.) or conditioned medium obtained at 24 hours. (FIG. 6E).

### Synthesis of EMNs and Neuroprotection Assay

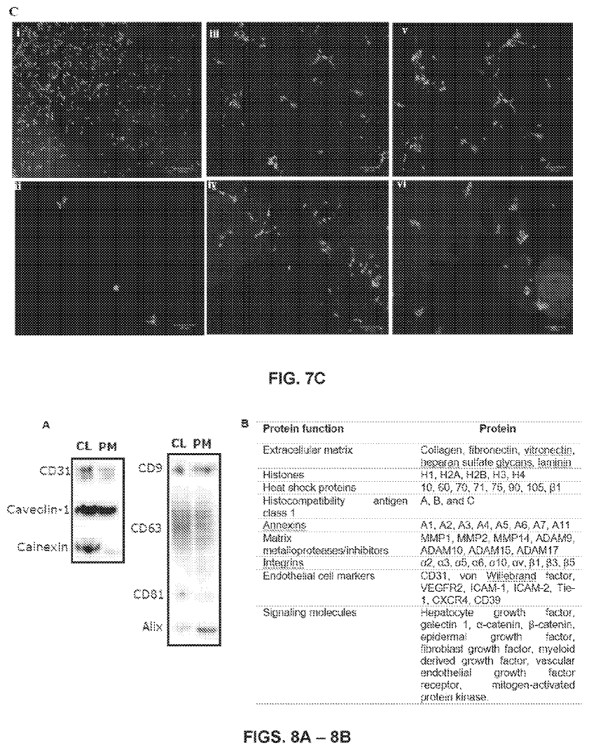

Using the optimal conditions standardized above, EMNs were loaded with 0.5 mg/mL concentrated conditioned medium had a size range of ˜135.7±4.8 nm and a concentration of ˜3.78×10⁹+/−1.05×10⁹ particles/ml (FIG. 7A). TEM images of the conditioned medium loaded EMNs displayed a circular morphology different from the characteristic cup-shaped morphology of native exosomes (FIG. 7B) (Thery et al., Curr Protoc Cell Biol, (2006)). The apoptotic SH-SY5Y cells were treated 1000, 2000, 4000 and 8000 EMNs/cell. The cells treated with 1000, 2000 and 4000 EMNs/cell showed an increase in the number of cells similar in morphology to normal SH-SY5Y cells when compared to the PBS-only treated cells that had more rounded morphology typical of dying apoptotic cells and a low number of surviving cells (FIG. 7C). Cells treated with 8000 EMN+CM/cell had more rounded cells suggesting a dose dependency in the neuroprotective function of EMNs loaded with hPMSC conditioned medium.

### Discussion

Neurological disorders affect people of varied age groups and manifest due to cellular dysfunction and death of neurons. Due to complexity of the neurological disease, treatments help manage the symptoms and no permanent cure has been identified. Owing to the ability of MSCs to differentiate into various functional tissues, stem cell therapies employing MSCs have gained popularity (Mahmoudifar et al., Methods Mol Biol (2015); Gardner et al., Methods Mol Biol, (2015); Phelps et al., Stem Cell International, (2018)). Studies have shown that the benefits of using MSC is mainly due to the biomolecules and growth factors these cells release into their extracellular environment (Shologu et al., Int J Mol Sci, (2018); Phan et al., Journal of Extracellular Vesicles, (2018); Venugopal et al., Curr Gene Ther, (2018)). Although MSC transplantation is widely used in the treatment of number of diseases, the administration of these cells result in graft rejection or limited and unintended engraftment (Gnecchi et al., Methods Mol Biol, (2016); Gnecchi et al., Nat Med, (2005); Lou et al., Exp Mol Med, (2017)), hence research is now focused on utilizing the MSC secretome containing secreted proteins and extracellular vesicles such as exosomes.

Exosomes are one of the principle cell-free treatment options that have been researched upon recently. Exosomes are 50-150 nm double-layered vesicles secreted by most cell type including B and T lymphocytes, mast cells, adipocytes and platelets (Lee et al., Int Immunopharmacol, (2012); Guo et al., Neuropsychiatr Dis Treat, (2017); van der Pol et al., Pharmacol Rev, (2012)) in response to internal and external changes. Recent studies have indicated that there exists a heterogeneity within the components present within native exosomes (Brenner et al., Methods Mol Biol, (2019)). The composition of the native exosome is known to be affected by the cell type, stress levels and health state of the cells from which the exosomes were isolated (Jelonek et al., Protein and Peptide Letters, (2016)). Previous work in the lab has shown that hPMSCs secrete significant levels of BDNF, VEGF and HGF in to the conditioned medium. Furthermore, a recent study by Kumar et al. (2019) showed that the hPMSC conditioned medium and exosomes when used to treat apoptotic neurons could improve the survival of the neurons (Kumar et al., (2019)). Despite serving as effective neuroprotective nanocarriers exosomes are secreted from cells in relatively low amounts; 0.1 μg from 1×10⁶ cells in a day (Thery et al., Curr Protoc Cell Biol, (2006)). In order to overcome the production and heterogeneity associated with native exosomes, this project was focused on packaging the neuroprotective hPMSC secretome into cell-derived lipid rafts to produce conditioned medium loaded artificial EMNs. Lipid rafts, the highly organized sections of the plasma membrane, are composed of cholesterol, sphingolipids and phospholipids (Pike et al., 2003). The surface of lipid rafts are composed of numerous cell surface receptors such as Caveolae and integrins that help in signal transduction (Pike, Journal of Lipid Research, (2003); Lingwood et al., Science (2010)), thus making lipid raft vesicles effective nanocarriers for neuroprotective secretion. The presence of the integrin and exosome-specific markers confirm that lipid rafts shared some of the markers of native exosomes and likely have the targeting potential associated with native exosomes. Owing to the presence of integrin α4 and β1, in the future the α4β1 receptor can be conjugated with ligands such as LLP2A to enhance targeting (Yao et al., Stem Cells, (2013)). As yield is an issue with using native exosomes, from this project Applicant noticed that the synthesis of EMNs could be scaled up and a total of 3.78×10⁹±1.05×10⁹ EMNs could be produced from 10×10⁶ hPMSCs (Jo Nanoscale, (2014)). A dose between 1000-4000 EMNs/cell was capable of either reversing the adverse effects of apoptosis or was able help in the proliferation of cells that could survive the apoptosis treatment. In the future Applicant could optimize the dosage and assess the neuroprotective function on primary neurons isolated from rat brains to determine whether this would be a feasible in vivo treatment. In order to visualize the mechanism of action of EMNs, Applicant could label the cells with palmitoylated GFP such that the EMNs so produced would carry the GFP signal and subsequently image them using fluorescent microscopy techniques (Lai et al., Nature Communications, (2015)).

In summary, in this project Applicant was able to show that the lipid raft section of the plasma membrane expresses the surface markers of exosomes that likely take part in homing and targeting. Since low yields are a potential problem in using native exosomes, Applicant showed that a greater number of EMNs can be produced with relatively low number of cells, thus overcoming the production and collection issues involved in using native exosomes. The produced EMNs were packaged with neuroprotective secretions from hPMSC and were able to rescue apoptotic neurons in culture. Therefore, this project serves as a proof-of-concept that a cell-derived nanovesicle system can be employed for the delivery of neuroprotective factors to apoptotic neurons. Additionally studies were performed confirming whether these results can be reproducibly applied. Further studies are performed to show that this delivery system can be extended to other cell and disease models. Finally, in a broader perspective, this study is expanded to cater to other diseases by modifying the cargo and model system used for the treatment.

TABLE 4
List of the materials used along with the supplier names

| Supply | Company |
|---|---|
| T150 flask | Falcon, Durham, NC, USA |
| Dulbecco's modified eagle's | Hyclone, South Logan, UT, USA |
| medium | |
| Fetal bovine serum | Thermo Fisher Scientific, Waltham, MA, |
| | USA |
| Fibroblast growth factor and | AdventBio, Elk Grove Village, IL, USA |
| epithelial growth factor | |
| Phosphate-buffered saline | Hyclone, South Logan, UT, USA |
| Trypsin | TrpLe, Gibco, Denmark |
| Trypan Blue | VWR International, Solon, OH, USA |
| Luna II automatic cell counter | Logo Biosystems, Annandale, VA, USA |
| Cell Scraper | Fisher Scientific, Rockford, IL, USA |
| MES | EMD Millipore, Billeria, MA, USA |
| NaCl | Fisher Scientific, Rockford, IL, USA |
| Triton-X-100 | Sigma, St. Louis, MO, USA |
| Protease inhibitor cocktail | Catalog no: P8340, Sigma, St. Louis, MO, |
| | USA |
| OptiPrepTM | Catalog no: D1556, Sigma, St. Louis, |
| | MO, USA |
| Dot blot apparatus | Catalog no: 84BR32827, Bio-Rad, |
| | Hercules, CA, USA |
| 20 mM Tris base | Bio-Rad, Hercules, CA, USA |
| Bovine serum albumin | BSA; Fisher Scientific, Geel, Belgium |
| Caveolin-1 | Catalog no: 3238, Cell Signaling |
| | Technologies, Beverly, MA, USA |
| Anti-Rabbit HRP secondary | Catalog no: 1858415, Fisher Scientific, |
| antibody | Rockford, IL, USA |
| Super signal west dura kit | Thermo Fisher Scientific, Rockford, IL, |
| | USA |
| Image LabTM software | Bio-Rad Laboratories, Hercules, CA |
| NuUPAGE LDS sample | Thermo Fisher Scientific, Waltham, MA, |
| loading buffer | USA |
| Novex protein standard | Life Technologies, Carlsbad, CA, USA |
| Ponceau stain | Catalog no: P7170, Sigma, St. Louis, MO, |
| | USA |
| ALIX | Catalog no: SAB4200476, Sigma, St. |
| | Louis, MO, USA |
| TSG101 | Catalog no: T5701, Sigma, St. Louis, MO, |
| | USA |
| Integrin α-4 | Catalog no: 8440, Cell Signaling |
| | Technologies, Beverly, MA, USA |
| Integrin β-1 | Catalog no: NBP2-36561, Novus |
| | Biologicals, Centennial CO, USA |
| Calnexin | Catalog no: 2433, Cell Signaling |
| | Technologies, Beverly, MA, USA |
| Flotillin-1 | Catalog no: 3253, Cell Signaling |
| | Technologies, Beverly, MA, USA |
| Mini-extruder | Avanti Polar Lipids Inc. Alabaster, AL, |
| | USA |
| Black Centrifuge Tubes | Fisher Scientific, Geel, Belgium |
| Pierce BSA depletion | Catalog no: 85160, Pierce Biotechnology, |
| column | Rockford, IL, USA |
| Imperial ™ stain | Catalog no: 24615, Thermo Fisher |
| | Scientific, Rockford, IL, USA |
| Nano drop 2000 | Fisher Scientific, Geel, Belgium |
| Ultracentrifuge tube | Catalog no: 355642, Beckman Coulter, |
| | Brea, CA, USA |
| SW 28 rotor | Beckman Coulter, Brea, CA, USA |
| Amicon Ultra-15 centrifugal 3 | Millipore Sigma, Burlington, MA, USA |
| kDa filter units | |
| HiTrapTM Blue HP albumin | Catalog no: 17041201, GE Healthcare |
| depletion kit | Bio-Sciences, Uppsala, Sweden |
| 4-12% Bis-Tris NuPAGE gel | Catalog no: NP0336, Life Technologies, |
| | Carlsbad, CA, USA |
| Nano Sight LM10 | Malvern, Malvern, UK |
| Formvar-carbon coated EM | Catalog no: 01700-F, Ted Pella, Redding, |
| grids | CA, USA |
| 25% Glutaraldehyde solution | Catalog no: G5882, Sigma Aldrich, St. |
| | Louis, MO, USA |
| Uranyl Oxalate | Catalog no: 21447-25, Polysciences Inc., |
| | Warrington, PA, USA |
| Methyl Cellulose | Catalog no: M-6385, Sigma Aldrich, St. |
| | Louis, MO, USA |
| Uranyl Oxalate | Catalog no: 22400, Electron Microscopy |
| | Sciences, Hatfield, PA, USA |
| CM120 transmission electron | Philips/FEI BioTwin, Amsterdam, |
| microscope | Netherlands |
| SH-SY5Y neuroblastoma cells | American Type Culture Collection, |
| | Manassas, VA, USA |
| 8-welled permanox chamber | Catalog no: 177402, Thermo Fisher |
| slides | Scientific, Waltham, MA, USA |
| Staurosporine | Cell Signaling Technology Inc., Danvers, |
| | MA, USA |
| Calcein AM | Thermo Fisher Scientific, Waltham, MA, |
| | USA |

### Experiment No. 2—EMNs in Treating Endothelial Progenitor Cells (EPCs)

### EPC-EM Synthesis

### PLGA Nanoparticle Fabrication

PLGA nanoparticles were synthesized using a nanoprecipitation method. 1 mg/mL of 50:50 (lactide:glycolide) PLGA was dissolved in acetone. The PLGA solution was added slowly and dropwise to 3 mL of deionized water in a 50 mL beaker under stirring at 800 RPM. The solution was allowed to stir for 2 hours under open air at room temperature to allow excess acetone to evaporate. Following stirring, PLGA nanoparticles were collected and purified by ultrafiltration using Amicon® ultrafiltration tubes with a 10 kDa cutoff. The PLGA is rinsed three times with deionized water to remove excess organic solvent. Final PLGA nanoparticles were resuspended in deionized water at a concentration of 1 mg/mL and stored at 4° C. until further use.

miR126 Loading

microRNA mimic hsa-miR126-3p was mixed with spermidine, a cationic counterion, at a 15:1 N/P ratio for 15 minutes at room temperature in nuclease-free water in order to create neutral complexes that improve nucleotide stability. The miR126 was then be added to the PLGA in acetone solution during the first step of PLGA nanoparticle synthesis. The miR126/PLGA mixture was vortexed vigorously for 30 seconds and then added dropwise to 3 mL of deionized water under constant stirring to ensure homogenous particle formation. Rest of the synthesis procedure was proceed as described above.

### Plasma Membrane (PM) Isolation

Umbilical cord-derived EPCs at passage 5 were seeded in 10 T150 flasks and grown in endothelial growth medium, 5% fetal bovine serum, and all growth factors as purchased from PromoCell®. At 90% confluency, cells were scraped off the flasks and collected in ice-cold 50 mL conical tubes. The cells were centrifuged at 500×g for 5 minutes and the subsequent pellet was washed 2 times with ice-cold 1× PBS. Cells were then be resuspended in 4 mL of hypotonic lysis buffer (20 mM Tris-HCl, pH=7.5, 10 mM KCl, 2 mM MgCl₂) with 5 μL of protease inhibitor cocktail to preserve protein function and incubated on ice for 1 hour. The cell lysate were homogenized on ice using a Dounce homogenizer for 30 passes and then incubated on ice for 5 minutes. The homogenized lysate was ultracentrifuged at 10,000×g at 4° C. for 20 minutes to pellet the cell nuclei and other organelles. The pellet was discarded and the supernatant was ultracentrifuged at 100,000×g at 4° C. for 35 minutes. The resulting pellet was the plasma membrane fraction and was resuspended in 1× PBS at a concentration of 1 mg/mL, and stored at −80° C.

### EPC EM Synthesis

PM and PLGA cores were mixed together at different PM:PLGA ratios (0:1, 0.25:1, 0.5:1, 1:1, 1:0.5, 1:0.25, and 1:0) in deionized water for a total volume of 1 mL. The combined solution was coextruded through a 200 nm polycarbonate membrane using the Avanti MiniExtruder for 15 passes. The resulting EPC-EMs were centrifuged at 9,500 rpm for 20 minutes to remove excess PM fragments and passed through a 0.2 μm filter to remove any contaminants.

### SILY Functionalization

SILY-azide was conjugated to the PM coating of the EPC-EM using a combination of sulfo-NHS ester chemistry and copper-free Click chemistry. This conjugation was mediated by the biochemical linker dibenzocyclooctyne-sulfo-N-hydroxysuccinimidyl ester (DBCO-sulfo-NHS). DBCO-sulfo-NHS was prepared at a 1 mg/mL solution in PBS and mixed with EPC-EMs for a 40× molar excess of DBCO. The DBCO-sulfo-NHS/EM solution was incubated on a shaker at room temperature for 1 hour. The excess DBCO-sulfo-NHS was neutralized by reacting with Tris-HCl, pH 8 and removed using ultrafiltration. The azide-SILY was then added to the DBCO-EM conjugate at a 2:1 (for example weight ratio) azide:DBCO molar ratio and incubated overnight at 4° C. Excess azide-SILY was removed using dialysis tubing with a 14 kDa cutoff for 24 hours at 4° C.

### Exemplary Protocol

Part 1: PLGA Synthesis
1. Dissolve 1 mg/mL in of PLGA in acetone. For labelled NP, add 0.05 wt % of DiO dye.
2. Add dropwise to 3 mL of MilliQ water while stirring.
3. Stir at room temperature for 3 hours.
4. Load solution into Amicon ultracentrifuge tubes with 10 kDa cutoff. Centrifuge at 9500 rpm for 5 min.
5. Wash twice with 500 uL of MilliQ water.
6. Resuspend in 1 mL MilliQ water and store at 4 C or can lyophilize and resuspend later.

Part 2: PM Isolation
1. Detach the cells from the flask by scraping.
2. Add cold PBS and centrifuge at 500×g for 10 min.
3. Wash 2× with PBS.
4. Suspend cells in 4 mL hypotonic solution (20 mM Trist-HCl pH=7.5, 10 mM KCl, 2 mM MgCl2, 5 uL protease inhibitor cocktail). Incubate on ice for 30 minutes.
5. Homogenize with Dounce homogenizer (30 times) on ice.
6. Centrifuge at 3200×g for 5 min.
7. Pool the supernatants and centrifuge at 10,000×g for 20 min.
8. Discard pellet and centrifuge the supernatant at 100,000×g for 35 min.
9. Resuspend pellet in PBS and store at −80 C.

Part 3: EM Synthesis
1. Extrude plasma membrane through a 400 nm polycarbonate membrane.
2. Mix the plasma membrane solution with the PLGA NPs and extrude through 200 nm polycarbonate membrane.
3. Centrifuge at 9500×g for 4 min to remove extra PM and filter through 0.2 um syringe to remove contaminants.

Part 4: SILY Modification
1. Suspend EPC EM in PBS.
2. Prepare 10 mM of DBCO-sulfo-NHS in PBS or DMSO
3. Add 40-fold molar excess of DBCO-sulfo-NHS solution to cell membrane. Final concentration of DBCO-sulfo-NHS should be between 0.5-2 mM
4. Incubate reaction at room temperature for 30 minutes or on ice for 2 hours.
5. Add Tris-HCl pH 8 for final concentration of 75 mM.
6. Incubate at room temperature for 5 min or on ice for 15 min.
7. Remove unreacted DBCO-sulfo-NHS by spinning and washing 3× with Tris-HCl 10 kDa ultrafiltration tubes 4×. After last wash, resuspend in PBS.
8. Suspend azide-sample in PBS.
9. Add DBCO-EPC-EM conjugate to azide sample.
10. Incubate at room temperature for 4-12 hours or 4C for 2-12 hours—place on rotator.
11. Purify using a dialysis membrane with 15 kDa cutoff for 24 hours.

### EPC-EMs Can Be Successfully Synthesized and Exhibit EV-Mimicking Characteristics

Plasma membrane (PM) was successfully isolated from cord-blood derived EPCs using a combination of hypotonic lysis, mechanical homogenization, and serial ultracentrifugation. Western blot analysis (FIG. 8A) revealed presence of the plasma membrane marker caveolin-1 and the diminished presence of the endoplasmic reticulum marker calnexin (negative control). EPC surface marker CD31 was detected, indicating a preservation of parent cell identity. Finally, common EV markers of CD9, CD63, CD81, and ALIX were retained on the plasma membrane surface, indicating physical similarity to EV membrane structure. Next, proteomic analysis of isolated plasma membrane was conducted using tandem mass spectrophotometry. A total of 3472 proteins in 2781 clusters were identified using cluster analysis via Scaffold software (FIG. 8B). Vital signaling molecules and proteins were found to be present such as VEGFR2, mitogen-activated protein kinases, hepatocyte growth factor, epidermal growth factor, fibroblast growth factor, and galectin-1. Pathway analysis using the Search Tool for the Retrieval of Interacting Genes/Proteins (STRING) database revealed that the detected protein clusters were involved in biological processes relating to vesicle mediated transport, immune cell-mediated immunity, angiogenesis, and hemostasis (data not shown).

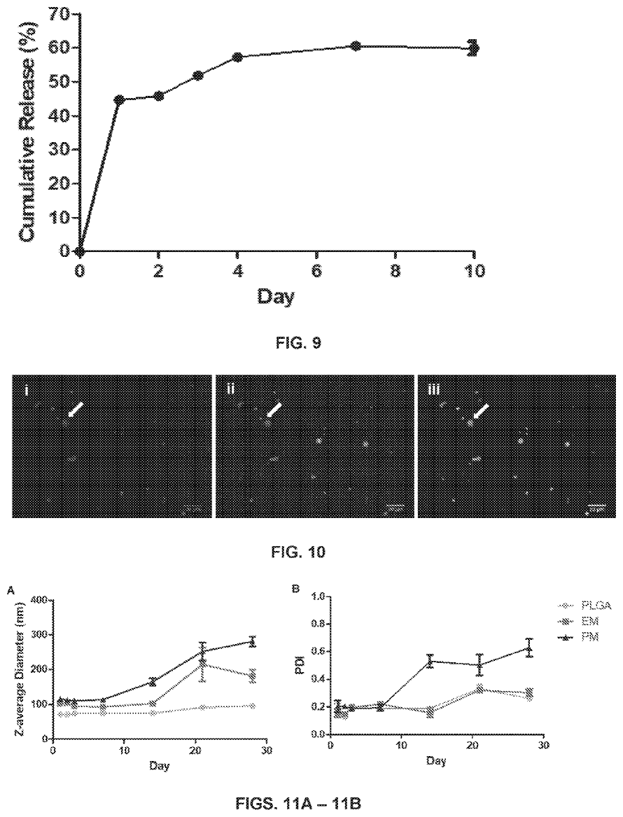

PLGA nanoparticles loaded with miR126 were synthesized using a modified nanoprecipitation method (Niu et al., Drug Development and Industrial Pharmacy, (2009)). These particles were found to be highly homogenous, with an average size of 77.11±12.1 nm (comparable to empty PLGA nanoparticles which were measured to be 71.5±0.325 nm) and a loading efficiency of 44.4%±3.5. Preliminary release kinetics studies revealed a burst release of miR126 from PLGA nanoparticles followed by a sustained release profile (FIG. 9). A 44% miRNA release was observed on Day 1 followed by slower sustained release over the next nine days. A cumulative release of about 60% was released over a period of 10 days.

Following successful PLGA nanoparticle synthesis and PM isolation, EPC-EMs were synthesized by coating PM around the PLGA particles. Florescence microscopy used to confirm the coating (FIG. 10). For visualization, PLGA particles were loaded with 1,1′-dioctadecyl-3,3,3′,3′-tetramethylindocarbocyanine (DiI) (excitation: 549 nm, emission: 565 nm), a red dye, while the PM was labeled green using PKH67 (excitation: 490 nm, emission: 502 nm). Composite images show their colocalization of the two as yellow particles, confirming the coating.

Various PM:PLGA ratios were tested and the ratio was seen to impact size and stability of the EPC-EM (data not shown). Higher PM:PLGA ratio improves EPC-EM stability, with a 2:1 weight ratio being optimal. At this ratio, EPC-EM size was 112.3±1.6 nm, which was comparable to native EPC EV size which was measured to be 113.5±9.1 nm. The membrane thickness at this ratio is estimated to be about 21 nm. The stability of EPC-EMs was assessed by monitoring the hydrodynamic size of the particles in water at 4° C. over a period of 28 days (FIG. 11). Dynamic light scattering revealed size and polydispersity index changes between PLGA nanoparticles, EMs, and PM vesicles over time. Addition of a PLGA core decreases particle aggregation and indicates improved stability as indicated by the limited increase and size and PDI for the EPC-EMs. Meanwhile, the PM vesicles, which contained no PLGA core, steadily increased in size and PDI, suggesting vesicle aggregation over time.

### SILY Can Be Bioconjugated to Isolated Plasma Membranes to Functionalize the Surface of EPC-EMs.

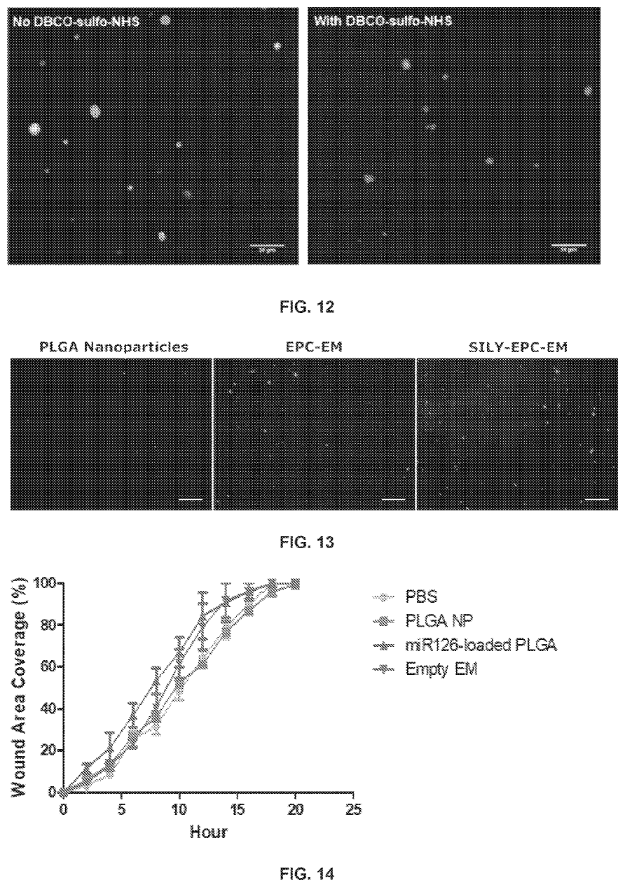

SILY (RRANAALKAGELYKSILYGC, SEQ ID NO: 1) is a platelet-derived peptide that has been shown to have strong binding affinity to collagen. Previously, SILY has been conjugated to poly(NIPAm-MBA-AMPS-AAc) nanoparticles in order facilitate binding to exposed collagen at the damaged sites (McMasters et al., AAPS J, (2015)). Similar principles were applied to Applicant's proposed EPC-EMs, where SILY was conjugated to the PM shell of the EPC-EM particles. Copper-free Click chemistry was used to link SILY to the PM shell. Click chemistry is a mild biochemical reactions used to covalently bind an azide group to an alkyne moiety using a triazole linkage (Presolski et al., Curr Protoc Chem Biol, (2011); Bonnet et al., Bioconjugate Chem., (2006)). A popular method of bioconjugation, Click chemistry has been often used to functionalize EV surfaces with a variety of peptides (Smyth et al., Bioconjug Chem., (2014); Jia et al., Biomaterials, (2018)). A proof-of-concept study was conducted to validate the use of DBCO-sulfo-NHS as a biochemical linker to conjugate a modified azide-SILY to PM via sulfo-NHS ester and Click chemistry. An azide-Cy5 dye (excitation: 647 nm, emission: 665 nm) was used as a proof-of-concept molecule in place of azide-SILY. Fluorescence microscopy confirmed strong conjugation to the EPC PM in presence of the DBCO-sulfo-NHS (FIG. 12).

CD39 and SILY can modulate collagen-mediated platelet adhesion and activation. Applicant confirmed that SILY-modified EPC-EMs were also able to bind to collagen surfaces under peristaltic conditions (FIG. 13). Fluorescent PLGA nanoparticles, EPC-EM, and SILY-EPC-EMs particles were flowed through collagen-coated channels on Ibidi slides at a shear stress of 15 dynes/cm2, to mimic the shear stress found in coronary arteries (Mongrain et al., Revista Espanola de Cardiologia (English Edition), (2006)). Bound particles were visualized using fluorescence microscopy. Fluorescent SILY-EPC-EMs were found to have remain bound to the collagen, suggesting that they can similarly target and bind exposed collagen at sites of endothelial damage. Moderate unmodified EPC-EM particle binding was also seen compared to uncoated PLGA nanoparticles, suggesting that there are functional collagen receptors on the PM that may additionally facilitate particle binding to collagen.

SILY is derived from a platelet receptor and has been hypothesized to block platelet adhesion by competitively binding to collagen (McMasters, Acta Biomaterialia, (2017); McMasters et al., AAPS J, (2015)). This suggests that SILY may also play a functional role in the design of Applicant's EPC-EMs by blocking platelet adhesion. While the SILY provides a physical barrier against platelets, the EPC PM can additionally provide a biological mechanism of platelet inhibition. Endothelial cells constitutively express CD39 which has been found to be a highly effective inhibitor of platelet reactivity (Marcus et al., Ital Heart J, (2001)). CD39, also known as NTPDase-1, has been shown to be a major mediator of platelet activation processes. It metabolically neutralizes ADP, a main prothrombotic component of platelet releasate, and thus prevents the activation of neighboring platelets. Applicant's previous proteomics data identified the presence of CD39 on the isolated EPC PM (FIG. 8B). This leads us to hypothesize that the EPC PM will augment the role of SILY by aiding in inhibiting platelet activation and aggregation to further prevent thrombosis.

miR126 Cargo and PM Coating Play Functional Roles in EPC-EM Properties

The miR126-loaded PLGA nanoparticles were seen to promote EPC migration in a scratch assay, with EPCs migration increasing about 20% in comparison to control PBS and vehicle controls (FIG. 14). This suggests that the PLGA nanoparticles were able to successfully release miR126 and the loaded miR126 can remain functional to recruit progenitor cells, as was previously suggested in literature Additionally, Applicant found that treatment with empty EPC-EM (PM-coated PLGA particles without any loaded miR126) also significantly improves EPC migration. This suggests that the PM coating retains some functional properties that could have dramatic implications for therapeutic use.

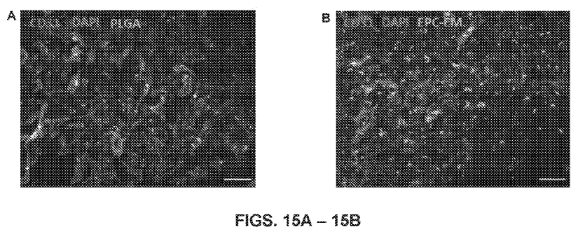

The uptake of particles was additionally further enhanced with PM coating. Fluorescent uncoated PLGA nanoparticles or EPC-EMs were incubated with EPCs for 24 hours, after which the particles were removed, and the cells were fixed and stained for cell nuclei and membrane markers (FIG. 15). Interestingly, Applicant also found that EPC-EMs tended to localize and aggregate within the perinuclear region, suggesting that particles are internalized via endocytosis.

Synthetic EVs provide an engineering solution by which a nanoparticle-based system can be designed to recapitulate the major functions of native EVs while still being able to be mass-produced and standardized. Here, without wishing to be bound by the theory, Applicant proposed that EVs can be mimicked by coating a cargo-loaded polymer core with a cell plasma membrane that is functionalized with different peptides of interest. Applicant validate this platform by engineering synthetic EPC-EMs in order to mimic EPC EVs. Applicant designed the components of the EPC-EMs to recapitulate physical (e.g. size, surface markers) and functional properties (e.g. angiogenesis) of native EPC EVs. Applicant further augmented the functional properties of the mimic with the conjugation of tailored peptides (e.g. SILY) to the surface of the plasma membrane coating. The successful validation of this system can lead to the establishment of a new nanotherapeutic platform that can reliably mimic native EVs. Different components of this EPC-EM system can be easily interchanged or substituted in order to develop new, unique disease-specific treatments. For example, other types of polymer cores (e.g. silica, alginate, cellulose, pullulan, gelatin, chitosan), different cellular plasma membranes origins (e.g. cancer cells, immune cells), variety of cargo (small molecules, DNA, RNA, proteins), and peptides (cell-penetrating peptides, cell-targeting peptides) can all be combined in various ways to develop personalized EV mimics. Thus, overall, this platform can be leveraged as an engineered alternative for the treatment of different types of injuries, diseases, and disorders.

The loading efficacy/encapsulation efficiency and/or yield was measure. One representative result show that encapsulation efficiency=(44×10⁻¹²) mol/mg PLGA×6.02×10²³ copies/mol=8.4×10¹² copies/mg PLGA. While 1 mg PLGA=2.53×10⁹ particles (n=1), 8.4×10¹² copies/mg PLGA)×1 mg/2.53×10⁹ particles×10⁶=3.3×10⁹ copies per 10⁶ particle. In one embodiment, the yield is 2.53×10⁹ particles/mL for 1 mg of PLGA.

### Experiment No. 3—EMNs Production

EMNS are also being produced from isolated lipid rafts or isolated plasma membrane as a shell. Such isolated lipid rafts and/or plasma membrane are derived from a cell selected from a differentiated cell, a stem cell (such as an adult stem cell, an embryonic stem cell, a neuronal stem cell, an endothelial progenitor cell (EPC), a cord-blood derived EPC, a mesenchymal stem cell, an adipose derived stem cell, a bone marrow derived stem cell, a placental-derived MSC (PMSC), or an induced pluripotent stem cell (iPSC)), an endothelial cell, a neuron, an astrocyte, an oligodendrocyte, an olfactory ensheathing cell, a microglial cell, a tumor cell, a cancer cell, an immune cell, a neutrophil, an eosinophil, a basophil, a mast cell, a monocyte, a macrophage, a dendritic cell, a natural killer cell, a lymphocyte, a B cell or a T cell, an animal cell, a mammalian cell, a human cell, or any combination thereof. Additionally or alternatively, the lipid rafts or plasma membrane are derived from a cell whose dysfunction causes a disease, for example, a neuron (dysfunctions of which lead to a neurological disorder), a motor neuron, a microglial cell (dysfunctions of which may also lead to a neurological disorder), a lung cell (dysfunction of which causes hypoxia and even death), or an epithelial cell (relating to a vascular disease).

Other cargos are loaded to the EMNs by themselves and/or combined with each other, including a conditioned medium derived from any cell, a peptide or protein (such as HGF, BDNF, VEGF, BMPs, CNTF, EGF, M-CSF, G-CSF, GM-CSF, Ephrin A1, Ephrin A2, Ephrin A3, Ephrin A4, Ephrin A5, Ephrin B1, Ephrin B2, Ephrin B3, EPO, FGF, GDF9, HDGF, Insulin-like growth factors, Interleukin, KGF, MSF, MSP, Neuregulin, NGF, NT-3, NT-4, PGF, PDGF, TCGF, TPO, TGF-α, TGF-β, or TNF-α), a polynucleotide (for example, a RNA, a DNA, an inhibitory RNA, an miRNA (such as hsa-miR-138-5p, hsa-miR-22-5p, miR-218-5p, hsa-let-7b-5p, hsa-let-7f-5p, hsa-miR-122-5p, hsa-let-7g-5p, hsa-let-7i-5p, hsa-miR-22-5p, hsa-miR-186-5p, hsa-let-7d-5p, hsa-miR-19a-3p, hsa-mir-98, hsa-let-7c, or hsa-miR-29a-3p), an siRNA, a therapeutic gene or a CRISPR system).

One or more of the cargos are loaded to a core both of which are encapsulated in a shell. Such core may be selected from poly(l-lysine) (PLL), polyethylenimine (PEI), polyamidoamines, polyimidazoles, poly(ethylene oxide), polyalkylcyanoacrylates, polylactide, polylactic acid (PLA), poly-ε-caprolactone (PCL), poly (lactic-co-glycolic acid) (PLGA), silica, alginate, cellulose, pullulan, gelatin, or chitosan.

### Experiment No. 4—EMNs for Treating Spinal Cord Injury

This example describes an exemplary method for treating spinal cord injury in a subject. A subject diagnostic with or suspect of having spinal cord injury is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of spinal cord injury as well as the tested treatment therein as an EMN cargo may be utilized: Liu et al. (2019); Wang et al. (2019); and Liu et al. (2020).

### Experiment No. 5—EMNs for Traumatic Brain Injury

This example describes an exemplary method for treating traumatic brain injury in a subject. A subject diagnostic with or suspect of having traumatic brain injury is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of traumatic brain injury as well as the tested treatment therein as an EMN cargo may be utilized: Xiong et al. (2017), NIH sponsored program R01-NS100710-01A1 accessed at grantome.com/grant/NIH/R01-NS100710-01A1, Ni et al, (2019), and Yang et al. (2017).

### Experiment No. 6—EMNs for Treating Stroke

This example describes an exemplary method for treating stroke in a subject. A subject diagnostic with or suspect of having stroke is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of stroke as well as the tested treatment therein as an EMN cargo may be utilized: Chen et al. (2016) and Spellicy et al. (2019).

### Experiment No. 7—EMNs for Treating Alzheimer'S Disease

This example describes an exemplary method for treating Alzheimer's disease in a subject. A subject diagnostic with or suspect of having Alzheimer's disease is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of Alzheimer's disease as well as the tested treatment therein as an EMN cargo may be utilized: Reza-Zaldivar et al. (2018); and Reza-Zaldivar et al. (2019s).

### Experiment No. 8—EMNs for Treating Parkinson'S Disease

This example describes an exemplary method for treating Parkinson's disease in a subject. A subject diagnostic with or suspect of having Parkinson's disease is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of Parkinson's disease as well as the tested treatment therein as an EMN cargo may be utilized: Vilaca-Faria et al. (2019) and Haney et al. (2015).

### Experiment No. 9—EMNs for Treating Multiple Sclerosis

This example describes an exemplary method for treating multiple sclerosis in a subject. A subject diagnostic with or suspect of having multiple sclerosis is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of multiple sclerosis as well as the tested treatment therein as an EMN cargo may be utilized: Clark et al. (2019) and Chen et al. (2017).

### Experiment No. 10—EMNs for Treating Spina Bifida

This example describes an exemplary method for treating spina bifida in a subject. A subject diagnostic with or suspect of having spina bifida is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of spina bifida as well as the tested treatment therein as an EMN cargo may be utilized: Chen et al. (2017).

### Experiment No. 11—EMNs for Treating Hind Limb Ischemia

This example describes an exemplary method for treating hind limb ischemia in a subject. A subject diagnostic with or suspect of having hind limb ischemia is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of hind limb ischemia as well as the tested treatment therein as an EMN cargo may be utilized: Zhang K et al. (2019), Zhang K et al. (2018), and Han et al. (2019).

### Experiment No. 12—EMNs for Treating Cardiac Ischemia

This example describes an exemplary method for treating cardiac ischemia in a subject. A subject diagnostic with or suspect of having cardiac ischemia is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. One or more of the following models of cardiac ischemia as well as the tested treatment therein as an EMN cargo may be utilized: Wang et al. (2018), Lai et al. (2010); and Zhu et al. (2018).

### Experiment No. 12—EMNs for Treating Hyper-Inflammation

This example describes an exemplary method for treating hyper-inflammation in a subject. A subject diagnostic with or suspect of having hyper-inflammation is administered an effective amount of any EMN as disclosed herein including those produced as described in Example 3 via inhalation, intrathecal, epidural, intraspinal, oral, intranasal, intrapulmonary, intravenous, intraamniotic fluid and/or other suitable administration. In certain cell/animal models and/or subject, such hyper-inflammation may be caused by an infection, for example a coronavirus infection. In other cell/animal models and/or subject, the hyper-inflammation is caused by treatment with an antibody therapy, a cell therapy (such as administering CAR-T cells) and/or a gene therapy (such as administering an AAV viral vector). In a further embodiment, the cargo of the EMN is a polypeptide/protein, polynucleotide, a small molecular, and/or a therapeutic agent, which modulates immune responses and/or is neuronal protective.

### Equivalents

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

### REFERENCES

Alonso, M. A. and J. Millán, The role of lipid rafts in signalling and membrane trafficking in T lymphocytes. Journal of Cell Science, 2001. 114(22): p. 3957%! The role of lipid rafts in signalling and membrane trafficking in T lymphocytes.
Ananta, J. S.; Paulmurugan, R.; Massoud, T. F. Nanoparticle-Delivered Antisense MicroRNA-21 Enhances the Effects of Temozolomide on Glioblastoma Cells. Mol. Pharmaceutics 2015, 12, 4509-4517.
Andrieu et al. (2019): Phage-displayed peptides targeting specific tissues and organs. J Drug Target. 2019 Jun.-Jul.; 27(5-6):555-565. doi: 10.1080/1061186X.2018.1531419. Epub 2018 Oct. 24.
Bai, L., et al., Hepatocyte growth factor mediates mesenchymal stem cell-induced recovery in multiple sclerosis models. Nat Neurosci, 2012. 15(6): p. 862-70.
Bonnet, D.; Ilien, B.; Galzi, J.-L.; Riché, S.; Antheaune, C.; Hibert, M. A Rapid and Versatile Method to Label Receptor Ligands Using “Click” Chemistry: Validation with the Muscarinic M1 Antagonist Pirenzepine. Bioconjugate Chem. 2006, 17, 1618-1623.
Brenner, A. W., G. H. Su, and F. Momen-Heravi, Isolation of Extracellular Vesicles for Cancer Diagnosis and Functional Studies. Methods Mol Biol, 2019. 1882: p. 229-237.
Calzarossa, C., et al., Neurorescue effects and stem properties of chorionic villi and amniotic progenitor cells. Neuroscience, 2013. 234: p. 158-72%7 2013/01/08%8 March 27%! Neurorescue effects and stem properties of chorionic villi and amniotic progenitor cells.
Chen et al. (2016): Intravenous administration of xenogenic adipose-derived mesenchymal stem cells (ADMSC) and ADMSC-derived exosomes markedly reduced brain infarct volume and preserved neurological function in rat after acute ischemic stroke. Oncotarget. 2016 Nov. 15; 7(46):74537-74556. doi: 10.18632/oncotarget.12902.
Chen et al. (2017): Fetal surgical repair with placenta-derived mesenchymal stromal cell engineered patch in a rodent model of myelomeningocele. J Pediatr Surg. 2017 Oct. 12. pii: S0022-3468(17)30662-0. doi: 10.1016/j.jpedsurg.2017.10.040. (Epub ahead of print)
Cibelli et al. (2002): Parthenogenetic stem cells in nonhuman primates. Science. 2002 Feb. 1; 295(5556):819.
Clark et al. (2019): Placental Mesenchymal Stem Cell-Derived Extracellular Vesicles Promote Myelin Regeneration in an Animal Model of Multiple Sclerosis. Cells 2019, 8.
Colombo et al. (2014): Biogenesis, Secretion, and Intercellular Interactions of Exosomes and Other Extracellular Vesicles. Annual Review of Cell and Developmental Biology 2014, 30, 255-289.
de Gassart, A., et al., Lipid raft-associated protein sorting in exosomes. Blood, 2003. 102(13): p. 4336-44.
Deng, H.; Sun, C.; Sun, Y.; Li, H.; Yang, L.; Wu, D.; Gao, Q.; Jiang, X. Lipid, Protein, and MicroRNA Composition Within Mesenchymal Stem Cell-Derived Exosomes. Cellular Reprogramming 2018, 20, 178-186.
Devalliere, J.; Chang, W. G.; Andrejecsk, J. W.; Abrahimi, P.; Cheng, C. J.; Jane-wit, D.; Saltzman, W. M.; Pober, J. S. Sustained delivery of proangiogenic microRNA-132 by nanoparticle transfection improves endothelial cell transplantation. The FASEB Journal 2014, 28, 908-922.
Devulapally, R.; Sekar, N. M.; Sekar, T. V.; Foygel, K.; Massoud, T. F.; Willmann, J. K.; Paulmurugan, R. Polymer Nanoparticles Mediated Codelivery of AntimiR-10b and AntimiR-21 for Achieving Triple Negative Breast Cancer Therapy. ACS Nano 2015, 9, 2290-2302.
Fish, J. E.; Santoro, M. M.; Morton, S. U.; Yu, S.; Yeh, R.-F.; Wythe, J. D.; Ivey, K. N.; Bruneau, B. G.; Stainier, D. Y. R.; Srivastava, D. miR-126 Regulates Angiogenic Signaling and Vascular Integrity. Developmental Cell 2008, 15, 272-284.
Gardner, O. F., M. Alini, and M. J. Stoddart, Mesenchymal Stem Cells Derived from Human Bone Marrow. Methods Mol Biol, 2015. 1340: p. 41-52.
Gnecchi, M., et al., Paracrine action accounts for marked protection of ischemic heart by Akt-modified mesenchymal stem cells. Nat Med, 2005. 11(4): p. 367-8%7 2005/04/07%8 Apr %! Paracrine action accounts for marked protection of ischemic heart by Akt-modified mesenchymal stem cells.
Gnecchi, M., et al., Paracrine Mechanisms of Mesenchymal Stem Cells in Tissue Repair. Methods Mol Biol, 2016. 1416: p. 123-46%7 2016/05/30%! Paracrine Mechanisms of Mesenchymal Stem Cells in Tissue Repair.
Guo et al. (2017): Guo, S., Y. Zhen, and A. Wang, Transplantation of bone mesenchymal stem cells promotes angiogenesis and improves neurological function after traumatic brain injury in mouse. Neuropsychiatr Dis Treat. 2017 Nov. 6; 13:2757-2765. doi: 10.2147/NDT.S141534. eCollection 2017. Published 6 Nov. 2017
Gupta et al. (2014) Exosomes as mediators of neuroinflammation. J Neuroinflammation. 2014 Apr. 3; 11:68. doi: 10.1186/1742-2094-11-68.
Han et al. (2019): Delivery of miR-675 by stem cell-derived exosomes encapsulated in silk fibroin hydrogel prevents aging-induced vascular dysfunction in mouse hindlimb. Mater Sci Eng C Mater Biol Appl. 2019 June; 99:322-332. doi: 10.1016/j.msec.2019.01.122. Epub 2019 Jan. 28.
Haney et al. (2015): Exosomes as drug delivery vehicles for Parkinson's disease therapy. J Control Release. 2015 Jun. 10; 207:18-30. doi: 10.1016/j.jconrel.2015.03.033. Epub 2015 Mar. 31.
Izuhara, M.; Kuwabara, Y.; Saito, N.; Yamamoto, E.; Hakuno, D.; Nakashima, Y.; Horie, T.; Baba, O.; Nishiga, M.; Nakao, T.; et al. Prevention of neointimal formation using miRNA-126-containing nanoparticle-conjugated stents in a rabbit model. PLoS ONE 2017, 12, e0172798.
Jansen, F.; Stumpf, T.; Proebsting, S.; Franklin, B. S.; Wenzel, D.; Pfeifer, P.; Flender, A.; Schmitz, T.; Yang, X.; Fleischmann, B. K.; et al. Intercellular transfer of miR-126-3p by endothelial microparticles reduces vascular smooth muscle cell proliferation and limits neointima formation by inhibiting LRP6. Journal of Molecular and Cellular Cardiology 2017, 104, 43-52.
Jelonek, K., P. Widlak, and M. Pietrowska, The Influence of Ionizing Radiation on Exosome Composition, Secretion and Intercellular Communication. Protein and peptide letters, 2016. 23(7): p. 656-663.
Jia, G.; Han, Y.; An, Y.; Ding, Y.; He, C.; Wang, X.; Tang, Q. NRP-1 targeted and cargo-loaded exosomes facilitate simultaneous imaging and therapy of glioma in vitro and in vivo. Biomaterials 2018, 178, 302-316.
Jo, W., J. Kim, J. Yoon, D. Jeong, S. Cho, H. Jeong, Y. J. Yoon, S. C. Kim, Y. S. Gho and J. Park (2014). “Large-scale generation of cell-derived nanovesicles.” Nanoscale 6(20): 12056-12064
Joner, M.; Finn, A. V.; Farb, A.; Mont, E. K.; Kolodgie, F. D.; Ladich, E.; Kutys, R.; Skorija, K.; Gold, H. K.; Virmani, R. Pathology of drug-eluting stents in humans: delayed healing and late thrombotic risk. J. Am. Coll. Cardiol. 2006, 48, 193-202.
Jones, G. N., et al., Ontological differences in first compared to third trimester human fetal placental chorionic stem cells. PLoS One, 2012. 7(9): p. e43395.
Klinker et al. (2015) Mesenchymal stem cells in the treatment of inflammatory and autoimmune diseases in experimental animal models World J Stem Cells. 2015 Apr. 26; 7(3): 556-567. Published online 2015 Apr. 26. doi: 10.4252/wjsc.v7.i3.556
Kroschinsky F, Stolzel F, von Bonin S, Beutel G, Kochanek M, Kiehl M, Schellongowski P (April 2017). “New drugs, new toxicities: severe side effects of modern targeted and immunotherapy of cancer and their management”. Critical Care. 21 (1): 89. doi:10.1186/s13054-017-1678-1. PMC 5391608. PMID 28407743.
Kumar et al. (2019): Neuroprotective effect of placenta-derived mesenchymal stromal cells: role of exosomes. The FASEB Journal 2019, 33, 5836-5849.
Lai et al. (2010): Exosome secreted by MSC reduces myocardial ischemia/reperfusion injury. Stem Cell Res. 2010 May; 4(3):214-22. doi: 10.1016/j.scr.2009.12.003. Epub 2010 Jan. 4.
Lai, C. P., et al. Visualization and tracking of tumour extracellular vesicle delivery and RNA translation using multiplexed reporters. Nature Communications, 2015. 6: p. 7029.
Lankford, L., T. Selby, J. Becker, V. Ryzhuk, C. Long, D. Farmer and A. Wang (2015). “Early gestation chorionic villi-derived stromal cells for fetal tissue engineering.” World J Stem Cells 7(1): 195-207
Lee D W, Gardner R, Porter D L, Louis C U, Ahmed N, Jensen M, Grupp S A, Mackall C L (July 2014). “Current concepts in the diagnosis and management of cytokine release syndrome”. Blood. 124(2): 188-95. doi:10.1182/blood-2014-05-552729. PMC 4093680. PMID 24876563.
Lee, J. M., et al., Comparison of immunomodulatory effects of placenta mesenchymal stem cells with bone marrow and adipose mesenchymal stem cells. Int Immunopharmacol, 2012. 13(2): p. 219-24.
Li et al. (2016a), Li, X.; Chen, C.; Wei, L.; Li, Q.; Niu, X.; Xu, Y.; Wang, Y.; Zhao, J. Exosomes derived from endothelial progenitor cells attenuate vascular repair and accelerate reendothelialization by enhancing endothelial function. Cytotherapy 2016, 18, 253-262.
Li et al. (2016b) Li, X.; Jiang, C.; Zhao, J. Human endothelial progenitor cells-derived exosomes accelerate cutaneous wound healing in diabetic rats by promoting endothelial function. Journal of Diabetes and its Complications 2016, 30, 986-992.
Li et al., (2018), Exosomal cargo-loading and synthetic exosome-mimics as potential therapeutic tools. Acta Pharmacol Sin. 2018 April; 39(4):542-551. doi: 10.1038/aps.2017.178. Epub 2018 Jan. 25.
Li, X.; Corbett, A. L.; Taatizadeh, E.; Tasnim, N.; Little, J. P.; Garnis, C.; Daugaard, M.; Guns, E.; Hoorfar, M.; Li, I. T. S. Challenges and opportunities in exosome research—Perspectives from biology, engineering, and cancer therapy. APL Bioeng 2019, 3, 011503.
Liang, X., et al., Paracrine mechanisms of mesenchymal stem cell-based therapy: current status and perspectives. Cell Transplant, 2014. 23(9): p. 1045-59.
Lingwood, D. and K. Simons, Lipid rafts as a membrane-organizing principle. Science, 2010. 327(5961): p. 46-50.
Lou, G., et al., Mesenchymal stem cell-derived exosomes as a new therapeutic strategy for liver diseases. Exp Mol Med, 2017. 49(6): p. e346%7 2017/06/18%8 June 16%! Mesenchymal stem cell-derived exosomes as a new therapeutic strategy for liver diseases %@ 2092-6413 (Electronic) 1226-3613 (Linking).
Liu et al. (2019): Exosomes Derived from Bone Mesenchymal Stem Cells Repair Traumatic Spinal Cord Injury by Suppressing the Activation of Al Neurotoxic Reactive Astrocytes. J Neurotrauma. 2019 Feb. 1; 36(3):469-484. doi: 10.1089/neu.2018.5835. Epub 2018 Aug. 13.
Liu et al. (2020): Exosome-shuttled miR-216a-5p from hypoxic preconditioned mesenchymal stem cells repair traumatic spinal cord injury by shifting microglial M1/M2 polarization. J Neuroinflammation. 2020 Feb. 4; 17(1):47. doi: 10.1186/s12974-020-1726-7.
Lunn, J. S., et al., Stem Cell Technology for Neurodegenerative Diseases. Annals of neurology, 2011. 70(3): p. 353-361.
Mahmoudifar, N. and P. M. Doran, Mesenchymal Stem Cells Derived from Human Adipose Tissue. Methods Mol Biol, 2015. 1340: p. 53-64.
Malm, T., S. Loppi, and K. M. Kanninen, Exosomes in Alzheimer's disease. Neurochemistry International, 2016. 97: p. 193-199.
Marcus, A. J.; Broekman, M. J.; Drosopoulos, J. H.; Pinsky, D. J.; Islam, N.; Maliszewsk, C. R. Inhibition of platelet recruitment by endothelial cell CD39/ecto-ADPase: significance for occlusive vascular diseases. Ital Heart J 2001, 2, 824-830.
Mathiyalagan, P.; Liang, Y.; Kim, D.; Misener, S.; Thorne, T.; Kamide, C. E.; Klyachko, E.; Losordo, D. W.; Hajjar, R.; Sahoo, S. Angiogenic Mechanisms of Human CD34+ Stem Cell Exosomes in the Repair of Ischemic Hindlimb. Circ Res 2017, 120, 1466-1476.
McMasters, J.; Panitch, A. Collagen-binding nanoparticles for extracellular anti-inflammatory peptide delivery decrease platelet activation, promote endothelial migration, and suppress inflammation. Acta Biomaterialia 2017, 49, 78-88.
McMasters, J.; Panitch, A. Prevention of Collagen-Induced Platelet Binding and Activation by Thermosensitive Nanoparticles. AAPS J 2015, 17, 1117-1125.
Mirotsou, M., et al., Paracrine mechanisms of stem cell reparative and regenerative actions in the heart. Journal of molecular and cellular cardiology, 2011. 50(2): p. 280-289.
Mongrain, R.; Rodes-Cabau, J. Role of Shear Stress in Atherosclerosis and Restenosis After Coronary Stent Implantation. Revista Espanola de Cardiologia (English Edition) 2006, 59, 1-4.
Murphy, D. E.; de Jong, O. G.; Brouwer, M.; Wood, M. J.; Lavieu, G.; Schiffelers, R. M.; Vader, P. Extracellular vesicle-based therapeutics: natural versus engineered targeting and trafficking. Exp Mol Med 2019, 51, 1-12.
NIH sponsored program R01-NS100710-01A1 accessed at grantome.com/grant/NIH/R01-NS100710-01A1
Ni et al. (2019): Exosomes Derived From Bone Mesenchymal Stem Cells Ameliorate Early Inflammatory Responses Following Traumatic Brain Injury. Front Neurosci. 2019 Jan. 24; 13:14. doi: 10.3389/fnins.2019.00014. eCollection 2019.
Niu, X.; Zou, W.; Liu, C.; Zhang, N.; Fu, C. Modified nanoprecipitation method to fabricate DNA-loaded PLGA nanoparticles. Drug Development and Industrial Pharmacy 2009, 35, 1375-1383.
Oh et al. (2015): Oh, M. and J. E. Nör, The Perivascular Niche and Self-Renewal of Stem Cells. Frontiers in Physiology, 2015. 6(367).
Paderi, J. E.; Stuart, K.; Sturek, M.; Park, K.; Panitch, A. The inhibition of platelet adhesion and activation on collagen during balloon angioplasty by collagen-binding peptidoglycans. Biomaterials 2011, 32, 2516-2523.
Pashoutan Sarvar, D., K. Shamsasenjan, and P. Akbarzadehlaleh, Mesenchymal Stem Cell-Derived Exosomes: New Opportunity in Cell-Free Therapy. Adv Pharm Bull, 2016. 6(3): p. 293-299%7 2016/10/22%8 September %! Mesenchymal Stem Cell-Derived Exosomes: New Opportunity in Cell-Free Therapy %@ 2228-5881 (Print) 2228-5881 (Linking).
Phan, J., et al., Engineering mesenchymal stem cells to improve their exosome efficacy and yield for cell-free therapy. Journal of extracellular vesicles, 2018. 7(1): p. 1522236-1522236.
Phelps, J., et al., Bioprocessing of Mesenchymal Stem Cells and Their Derivatives: Toward Cell-Free Therapeutics. Stem Cells International, 2018. 2018: p. 23.
Pike, L. J., Lipid rafts: bringing order to chaos. Journal of Lipid Research, 2003. 44(4): p. 655-667.
Polyakova, M., et al., Stability of BDNF in Human Samples Stored Up to 6 Months and Correlations of Serum and EDTA-Plasma Concentrations. International journal of molecular sciences, 2017. 18(6): p. 1189.
Presolski, S. I.; Hong, V. P.; Finn, M. G. Copper-Catalyzed Azide-Alkyne Click Chemistry for Bioconjugation. Curr Protoc Chem Biol 2011, 3, 153-162.
Ramasubramanian et al. (2019): Engineering Extracellular Vesicles as Nanotherapeutics for Regenerative Medicine. Biomolecules. 2019 Dec. 28; 10(1). pii: E48. doi: 10.3390/biom10010048.
Reza-Zaldivar et al. (2018): Potential Effects of MSC-Derived Exosomes in Neuroplasticity in Alzheimer's Disease. Front Cell Neurosci. 2018 Sep. 24; 12:317. doi: 10.3389/fncel.2018.00317. eCollection 2018.
Reza-Zaldivar et al. (2019): Mesenchymal stem cell-derived exosomes promote neurogenesis and cognitive function recovery in a mouse model of Alzheimer's disease. Neural Regen Res. 2019 September; 14(9):1626-1634. doi: 10.4103/1673-5374.255978.
Sessa, R.; Seano, G.; di Blasio, L.; Gagliardi, P. A.; Isella, C.; Medico, E.; Cotelli, F.; Bussolino, F.; Primo, L. The miR-126 regulates angiopoietin-1 signaling and vessel maturation by targeting p85β3. Biochim. Biophys. Acta 2012, 1823, 1925-1935.
Sharma, S.; Parmar, A.; Kori, S.; Sandhir, R. PLGA-based nanoparticles: A new paradigm in biomedical applications. TrAC Trends in Analytical Chemistry 2016, 80, 30-40.
Shaw et al. (2017) Shaw, G., The Economic Burden of Neurologic Disease. Neurology Today, 2017. 17(12): p. 1, 14-18.
Shologu, N., et al., Human Mesenchymal Stem Cell Secretome from Bone Marrow or Adipose-Derived Tissue Sources for Treatment of Hypoxia-Induced Pulmonary Epithelial Injury. Int J Mol Sci, 2018. 19(10).
Smyth, T.; Petrova, K.; Payton, N. M.; Persaud, I.; Redzic, J. S.; Graner, M. W.; Smith-Jones, P.; Anchordoquy, T. J. Surface Functionalization of Exosomes Using Click Chemistry. Bioconjug Chem 2014, 25, 1777-1784.
Spellicy et al. (2019): Neural Stem Cell Extracellular Vesicles Disrupt Midline Shift Predictive Outcomes in Porcine Ischemic Stroke Model. Transl Stroke Res. 2019 Dec. 6. doi: 10.1007/s12975-019-00753-4.
Talwadekar, M. D., V. P. Kale, and L. S. Limaye, Placenta-derived mesenchymal stem cells possess better immunoregulatory properties compared to their cord-derived counterparts—a paired sample study. Scientific Reports, 2015. 5: p. 15784%8 10/28 07/13/received 10/01/accepted %! Placenta-derived mesenchymal stem cells possess better immunoregulatory properties compared to their cord-derived counterparts—a paired sample study %@ 2045-2322.
Thery, C., S. Amigorena, G. Raposo and A. Clayton (2006). Isolation and characterization of exosomes from cell culture supernatants and biological fluids.” Curr Protoc Cell Biol Chapter 3: Unit 3.22.
Théry et al. (2002): Exosomes: composition, biogenesis and function. Nat. Rev. Immunol. 2002, 2, 569-579.
Tran, T. H., et al., Exosomes as nanocarriers for immunotherapy of cancer and inflammatory diseases. Clin Immunol, 2015. 160(1): p. 46-58%7 2015/04/07%8 September %! Exosomes as nanocarriers for immunotherapy of cancer and inflammatory diseases.
Tsumaru, S.; Masumoto, H.; Minakata, K.; Izuhara, M.; Yamazaki, K.; Ikeda, T.; Ono, K.; Sakata, R.; Minatoya, K. Therapeutic angiogenesis by local sustained release of microRNA-126 using poly lactic-co-glycolic acid nanoparticles in murine hindlimb ischemia. Journal of Vascular Surgery 2018, 68, 1209-1215.
van der Pol, E., et al., Classification, functions, and clinical relevance of extracellular vesicles. Pharmacol Rev, 2012. 64(3): p. 676-705.
Varshney, P., V. Yadav, and N. Saini, Lipid rafts in immune signalling: current progress and future perspective. Immunology, 2016. 149(1): p. 13-24.
Venugopal, C., et al., Neuroprotection by Human Dental Pulp Mesenchymal Stem Cells: From Billions to Nano. Curr Gene Ther, 2018. 18(5): p. 307-323.
Vilaça-Faria et al. (2019): Mesenchymal Stem Cells-derived Exosomes: A New Possible Therapeutic Strategy for Parkinson's Disease? Cells. 2019 Feb. 2; 8(2). pii: E118. doi: 10.3390/cells8020118.
Villar et al. (2016) Localization and signaling of GPCRs in lipid rafts. Methods Cell Biol. 2016; 132:3-23. doi: 10.1016/bs.mcb.2015.11.008. Epub 2016 Feb. 10.
Vrana et al. (2003) Nonhuman primate parthenogenetic stem cells. Proc Natl Acad Sci USA. 2003 Sep. 30; 100 Suppl 1:11911-6. Epub 2003 Sep. 22.
Wang et al. (2015): Placental mesenchymal stromal cells rescue ambulation in ovine myelomeningocele. Stem Cells Transl Med, 2015. 4(6): p. 659-69.
Wang et al. (2018): Engineered Exosomes With Ischemic Myocardium-Targeting Peptide for Targeted Therapy in Myocardial Infarction. J Am Heart Assoc. 2018 Aug. 7; 7(15):e008737. doi: 10.1161/JAHA.118.008737.
Wang et al. (2019): Combinational Treatment of Bioscaffolds and Extracellular Vesicles in Spinal Cord Injury. Front Mol Neurosci. 2019 Apr. 12; 12:81. doi: 10.3389/fnmo1.2019.00081. eCollection 2019.
Wu, X.; Liu, Z.; Hu, L.; Gu, W.; Zhu, L. Exosomes derived from endothelial progenitor cells ameliorate acute lung injury by transferring miR-126. Experimental Cell Research 2018, 370, 13-23.
Xiong et al. (2017): Emerging potential of exosomes for treatment of traumatic brain injury. Neural Regen Res. 2017 January; 12(1):19-22. doi: 10.4103/1673-5374.198966.
Yang et al. (2017): MSCs-Derived Exosomes and Neuroinflammation, Neurogenesis and Therapy of Traumatic Brain Injury. Front Cell Neurosci. 2017 Feb. 28; 11:55. doi: 10.3389/fncel.2017.00055. eCollection 2017.
Yao, W., et al., Reversing bone loss by directing mesenchymal stem cells to bone. Stem Cells, 2013. 31(9): p. 2003-14.
Zhang et al. (2019): Exosomes: biogenesis, biologic function and clinical potential. Cell Biosci 2019, 9, 19.
Zhang K et al. (2018): Enhanced Therapeutic Effects of Mesenchymal Stem Cell-Derived Exosomes with an Injectable Hydrogel for Hindlimb Ischemia Treatment. ACS Appl Mater Interfaces. 2018 Sep. 12; 10(36):30081-30091. doi: 10.1021/acsami.8b08449. Epub 2018 Aug. 29.
Zhang K et al. (2019): Molecular Imaging of Therapeutic Effect of Mesenchymal Stem Cell-Derived Exosomes for Hindlimb Ischemia Treatment. Methods Mol Biol. 2019 Apr. 3. doi: 10.1007/7651_2019_221.
Zhou, Y.; Li, P.; Goodwin, A. J.; Cook, J. A.; Halushka, P. V.; Chang, E.; Fan, H. Exosomes from Endothelial Progenitor Cells Improve the Outcome of a Murine Model of Sepsis. Molecular Therapy 2018, 26, 1375-1384.Zhu et al. (2018): Hypoxia-elicited mesenchymal stem cell-derived exosomes facilitates cardiac repair through miR-125b-mediated prevention of cell death in myocardial infarction. Theranostics. 2018 Nov. 29; 8(22):6163-6177. doi: 10.7150/thno.28021. eCollection 2018.

1. An exosome mimicking nanovesicle (EMN) comprising a shell encapsulating a cargo, wherein the shell comprises a plasma membrane and wherein the EMN is substantially devoid of native exosomes.

2. The EMN of claim 1, wherein the EMN comprises a lipid raft.

3.-4. (canceled)

5. The EMN of claim 1, wherein the stem cell is an adult stem cell and/or an embryonic stem cell, and optionally wherein the stem cell is selected from a neuronal stem cell, an endothelial progenitor cell (EPC), a cord-blood derived EPC, a umbilical cord-derived EPCs, a mesenchymal stem cell, an adipose derived stem cell, a bone marrow derived stem cell, a placental-derived MSC (PMSC), or an induced pluripotent stem cell (iPSC), and further optionally wherein the mesenchymal stem cell expresses one or more of CD105⁺, CD90⁺, CD73⁺, CD44⁺ and CD29⁺ and CD184+, and/or optionally wherein the mesenchymal stem cell lacks one or more of hematopoietic markers, and further optionally wherein the hematopoietic markers are selected from the group of: CD31, CD34 and CD45.

6. The EMN of claim 5, wherein the stem cell is a mesenchymal stem cell that expresses one or more exosome specific markers selected from the group of CD9, CD63, ALIZ, TSG101, alpha 4 integrin, beta 1 integrin, and/or the stem cell is a mesenchymal stem cell lacks expression of calnexin.

7.-8. (canceled)

9. The EMN of claim 1, wherein the cargo comprises a cell derived conditioned medium.

10. (canceled)

11. The EMN of claim 1, wherein the cargo comprises an exogenous agent, optionally wherein the exogenous agent is selected from a polynucleotide, a peptide, a protein, an antibody fragment, a small molecule or a therapeutic agent.

12. The EMN of claim 11, wherein the polynucleotide is selected from a RNA, a DNA, an inhibitory RNA, an miRNA, an siRNA, a therapeutic gene or a CRISPR system, and optionally wherein the miRNA is one or more of the following: hsa-miR-138-5p, hsa-miR-22-5p, miR-218-5p, hsa-let-7b-5p, hsa-let-7f-5p, hsa-miR-122-5p, hsa-let-7g-5p, hsa-let-7i-5p, hsa-miR-22-5p, hsa-miR-186-5p, hsa-let-7d-5p, hsa-miR-19a-3p, hsa-mir-98, hsa-let-7c, or hsa-miR-29a-3p, optionally wherein cargo comprises a miRNA and a cationic counterion, optionally wherein the a cationic counterion is spermidine, optionally wherein, the cargo comprises a complex comprising an hsa-miR126-3p and a cationic counterion, optionally wherein the a cationic counterion is spermidine, optionally wherein the therapeutic gene is a polynucleotide less than about 5000 nt, and further optionally wherein the therapeutic agent is a polynucleotide encoding a B-cell lymphoma/leukemia 11A.

13. The EMN of claim 1, further comprising a core encapsulated in the shell with the cargo, optionally wherein the core is selected from the group of a polymer core, optionally wherein the core is selected from the group of poly(l-lysine) (PLL), polyethylenimine (PEI), polyamidoamines, polyimidazoles, poly(ethylene oxide), polyalkylcyanoacrylates, polylactide, polylactic acid (PLA), poly-ε-caprolactone (PCL), poly (lactic-co-glycolic acid) (PLGA), silica, alginate, cellulose, pullulan, gelatin, or chitosan and optionally wherein the core comprises a PLGA core and optionally wherein the plasma membrane to PLGA weight ratio is about 1:10 to about 10:1, optionally about 1:5, about 2:1, about 1:1, about 2:1, about 1:2, or about 1:4.

14.-17. (canceled)

18. The EMN of claim 1, wherein the shell further comprises a peptide or a protein for facilitating one or more of the following: targeting the EMN to a cell and/or tissue, penetrating a cell, modulating immunoregulatory activity, or protecting a cell selected from neurons, endothelial cells, lung cells or a combination thereof.

19. (canceled)

20. The EMN of claim 18, wherein the peptide or protein is selected from the following: a collagen-binding ligand, a platelet-receptor for collagen, an inhibitor of platelet reactivity, SILY (RRANAALKAGELYKSILYGC, SEQ ID NO: 1), CD39; a cell-penetrating peptide; a cell-targeting peptide; a human leukocyte antigen-G (HLA-G); Galectin1 or a combination thereof.

21. The EMN of claim 18, wherein the peptide or protein is conjugated to the shell covalently or non-covalently, directly or indirectly via a linker, optionally wherein the peptide or protein is conjugated to the shell via one or more of the following: Click chemistry, DOPE-PEG-peptide, DOPE-NHS-peptide chemistry, biotin-streptavidin linkage, or peptide-peptide linkage, optionally wherein the peptide or protein is conjugated via using hosphatidylethanolamines, such as DSPE, DMPE, DPPE, or DOPE, optionally wherein the peptide or protein is conjugated to the shell via biotin-streptavidin linkage or peptide-peptide linkage, optionally wherein the peptide or protein covalently binds an azide group to an alkyne moiety using a triazole linkage, and further optionally wherein DBCO-sulfo-NHS comprises a biochemical linker to conjugate a modified azide-SILY to the shell via sulfo-NHS ester and Click chemistry.

22. (canceled)

23. A plurality of EMNs of claim 1, wherein the shells or cargos are the same or different from each other or wherein the shells and cargos are the same or different from each other.

24. A composition comprising a carrier and an EMN of claim 1.

25. (canceled)

26. A method for rescuing a cell selected from the group of: a neuron, an endothelial cell, or a lung cell comprising administering an effective amount of an EMN of claim 1.

27.-28. (canceled)

29. A method for preventing or treating one or more of: vascular diseases, neuronal diseases, or a hyper-inflammation in a subject in need thereof comprising administering to a subject in need thereof an effective amount of an EMN of claim 1, optionally wherein the vascular diseases are selected from the group of hind limb ischemia or cardiac ischemia, optionally wherein the neuronal diseases are selected from the group of a neurodegenerative disease or disorder, an ischemic brain injury, stroke, a moderate or a catastrophic brain injury, a chemical neurotoxin exposure, a spinal cord injury, a traumatic brain injury, Alzheimer's disease, Parkinson's disease or a spinal cord contusion, spina bifida, myelomeningocele (MCC), multiple sclerosis, demyelination, oligodendroglia degeneration, lack of oligodendrocyte precursor cell (OPC) differentiation, or paralysis, optionally wherein the hyper-inflammation is caused by a viral, bacterial, fungal or parasitic infection, optionally wherein the infection is a coronavirus infection, further optionally wherein the coronavirus is selected from Severe acute respiratory syndrome (SARS) coronavirus (SARS-CoV), SARS-CoV-2 causing the novel coronavirus disease-2019 (COVID-19), or Middle East respiratory syndrome (MERS) coronavirus (MERS-CoV), optionally wherein the hyper-inflammation is caused by an acute respiratory distress syndrome (ARDS), a virus induced ARDS, a pneumonia, or a drug treatment, further optionally wherein the drug treatment is selected from administering an antibody or a fragment thereof, a gene therapy, or a cell therapy, yet further optionally wherein the gene therapy is an adeno-associated virus therapy, and optionally wherein the cell therapy is selected from the group of an adoptive T-cell therapy, an adoptive NK-cell therapy, or an adoptive macrophage therapy.

30.-32. (canceled)

33. A method for treating a damaged cell selected from neurons, endothelial cells, or lung cells, or preventing the cells from being damaged comprising contacting the cell with an effective amount of an EMN of claim 1.

34.-37. (canceled)

38. A kit comprising an EMN of claim 1, and optionally, reagents and instructions for use of one or more diagnostically, as a research tool or therapeutically.

39. A method of producing of an EMN of claim 1, the comprising:
(i) optionally hypotonically lyse cells selected from the group of: a differentiated cell; a stem cell; a cancer cell; or an immune cell: neutrophils, eosinophils, basophils, mast cells, monocytes, macrophages, dendritic cells, natural killer cells, and lymphocytes (B cells and T cells);
(ii) an optional mechanical homogenization;
(iii) isolate or purify the lipid rafts and/or plasma membrane from the cell, optionally via one or more of centrifugation, optionally at the same or different relative centrifugal forces, optionally using serial ultracentrifugation and collecting materials at the density of lipid rafts and/or plasma membrane; and
(iv) extrude the lipid rafts and/or plasma membrane with a solution comprising cargos using an extruder, optionally the extruder comprises a filter selected from an about 50 nm to 300 nm filter, optionally an about 200 nm filter, an about 150 nm filter, an about 100 nm filter, whereby generating EMNs comprising a cargo and lipid rafts and/or plasma membrane; or
(v) extrude the lipid rafts and/or plasma membrane using an extruder, optionally the extruder comprises a filter selected from an about 50 nm to 300 nm filter, optionally an about 200 nm filter, an about 150 nm filter, an about 100 nm filter, centrifuge the extruded materials, remove supernatant and resuspend the rest martials comprising lipid rafts and/or plasma membrane using a solution comprising cargos, whereby EMNs were self-assembled from the extruded lipid rafts and/or plasma membrane encapsulating a cargo.

40.-44. (canceled)

45. A kit comprising the EMN of claim 1, and instructions for use.

46. (canceled)

47. A composition for use in rescuing a cell selected from the group of: a neuron, an endothelial cell, or a lung cell, comprising an effective amount of an EMN of claim 1.

48.-58. (canceled)


SEQUENCE LISTING (3 sequences)

SEQ ID NO: 1 - protein, 20 aa, Artificial Sequence
  SILY
RRANAALKAGELYKSILYGC

SEQ ID NO: 2 - protein, 12 aa, Artificial Sequence
  cell targeting peptide
QPWLEQAYYSTF

SEQ ID NO: 3 - protein, 12 aa, Artificial Sequence
  cell targeting peptide
YPHIDSLGHWRR